(19) United States
(12) Patent Application Publication
(10) Pub. No.: US 2007/0122807 A1
(43) Pub. Date: May 31, 2007

(54) Posh polypeptides, complexes and related methods

(75) Inventors: Iris Alroy, Ness-Ziona (IL); Yuval Reiss, Kiriat-Ono (IL); Daniel N. Taglicht, Lapid (IL); Liora Yaar, Raanana (IL)

Correspondence Address: FISH & NEAVE IP GROUP;ROPES & GRAY LLP, ONE INTERNATIONAL PLACE, BOSTON, MA, 02110-2624, US

(73) Assignee: Proteologics, Inc., Orangeburg, NY (US)

(21) Appl. No.: 10/551,587

(22) PCT Filed: Apr. 5, 2004
(86) PCT No.: PCT/US04/10582

§ 371 Date: Jul. 20, 2006

Related U.S. Application Data
(60) Provisional application No. 60/460,526, filed on Apr. 3, 2003.
(60) Provisional application No. 60/475,825, filed on Jun. 3, 2003.

(30) Foreign Application Priority Data
Mar. 2, 2004 (US) PCT/US04/06308

(51) Int. Cl. C12Q 1/68; C07H 21/04; C12P 21/06; C12N 9/64

(52) U.S. Cl. 435/6; 435/69.1; 435/226; 435/320.1; 435/325; 536/23.2

(57) ABSTRACT

The application provides novel complexes of POSH polypeptides and POSH-associated proteins. The application also provides methods and compositions for treating POSH-associated diseases such as neurological disorders.

### RELATED APPLICATIONS

This application claims the benefit of priority of U.S. Provisional Application No. 60/460,526 filed 3 Apr. 2003 and 60/475,825 filed 3 Jun. 2003 and a PCT Application filed on March 2, 2004 (Attorney Docket No. PROL-PWO-024), in the name of Daniel N. Taglicht, Iris Alroy, Yuval Reiss, Liora Yaar, Danny Ben-Avraham, Shmuel Tuvia, and Tsvika Greener entitled “Posh Interacting Proteins and Related Methods.” The teachings of the referenced Applications are incorporated herein by reference in their entirety.

### BACKGROUND

Potential drug target validation involves determining whether a DNA, RNA or protein molecule is implicated in a disease process and is therefore a suitable target for development of new therapeutic drugs. Drug discovery, the process by which bioactive compounds are identified and characterized, is a critical step in the development of new treatments for human diseases. The landscape of drug discovery has changed dramatically due to the genomics revolution. DNA and protein sequences are yielding a host of new drug targets and an enormous amount of associated information.

The identification of genes and proteins involved in various disease states or key biological processes, such as inflammation and immune response, is a vital part of the drug design process. Many diseases and disorders could be treated or prevented by decreasing the expression of one or more genes involved in the molecular etiology of the condition if the appropriate molecular target could be identified and appropriate antagonists developed. For example, many human genetic diseases, such as Huntington's disease, and certain prion conditions, which are influenced by both genetic and epigenetic factors, result from the inappropriate activity of a polypeptide as opposed to the complete loss of its function. Accordingly, antagonizing the aberrant function of such mutant genes would provide a means of treatment. Additionally, infectious diseases such as HIV have been successfully treated with molecular antagonists targeted to specific essential retroviral proteins such as HIV protease or reverse transcriptase. Drug therapy strategies for treating such diseases and disorders have frequently employed molecular antagonists which target the polypeptide product of the disease gene(s). However, the discovery of relevant gene or protein targets is often difficult and time consuming.

One area of particular interest is the identification of host genes and proteins that are co-opted by viruses during the viral life cycle. The serious and incurable nature of many viral diseases, coupled with the high rate of mutations found in many viruses, makes the identification of antiviral agents a high priority for the improvement of world health. Genes and proteins involved in a viral life cycle are also appealing as a subject for investigation because such genes and proteins will typically have additional activities in the host cell and may play a role in other non-viral disease states.

Other areas of interest include the identification of genes and proteins involved in cancer, apoptosis and neural disorders (particularly those associated with apoptotic neurons, such as Alzheimer's disease).

It would be beneficial to identify proteins involved in one or more of these processes for use in, among other things, drug screening methods. Additionally, once a protein involved in one or more processes of interest has been identified, it is possible to identify proteins that associate, directly or indirectly, with the initially identified protein. Knowledge of interactors will provide insight into protein assemblages and pathways that participate in disease processes, and in many cases an interacting protein will have desirable properties for the targeting of therapeutics. In some cases, an interacting protein will already be known as a drug target, but in a different biological context. Thus, by identifying a suite of proteins that interact with an initially identified protein, it is possible to identify novel drug targets and new uses for previously known therapeutics.

### SUMMARY

In part, the application relates to the ubiquitin ligase, POSH (Plenty of SH3 domains), and the discovery of novel interactions between POSH and proteins that associate with POSH (termed “POSH-APs”). By providing novel POSH:POSH-AP interactions, the application provides, in part, methods for modulating a process that POSH participates in by targeting a POSH-AP or the POSH:POSH-AP interaction. Furthermore, by providing novel POSH:POSH-AP interactions, the application provides, in part, methods for modulating a process that a POSH-AP participates in by targeting POSH.

In certain embodiments, the application relates to an isolated, purified or recombinant complex comprising a POSH polypeptide and a POSH-associated protein (POSH-AP). In certain embodiments, the POSH-AP is HERPUD1. In certain preferred embodiments, the application relates to an isolated, purified or receombinant complex comprising a POSH polypeptide and ubiquitinated HERPUD1. In further preferred embodiments, the HERPUD1 is monoubiquitinated. In certain further embodiments, the application provides a method of identifying an agent to treat a neurological disorder, the method comprising identifying a test agent that disrupts a complex comprising a POSH polypeptide and a POSH-AP, such as HERPUD1. In certain embodiments, the application relates to a method comprising identifying a test agent that disrupts a complex comprising a POSH polypeptide and ubiquitinated HERPUD1, such as monoubiquitinated HERPUD1.

In additional embodiments, the application relates to an isolated, purified or recombinant ubiquitinated HERPUD1 polypeptide. In further embodiments, the application relates to an isolated, purified or recombinant monoubiquitinated HERPUD1 polypeptide. In other embodiments, the monoubiquitinated HERPUD1 is at least 20%, 25%, 30%, 35%, 40%, 45%, 50%, 55%, 60%, 65%, 70%, 75%, 80%, 85%, 90%, 95%, 97%, 98%, 99% or 100% free of polyubiquitinated HERPUD1.

The application additionally relates to a method of identifying an agent that modulates a HERPUD1 function, comprising (a) identifying an agent that modulates POSH and (b) testing the effect of the agent on a HERPUD1 function. In certain embodiments, the application relates to a method of evaluating an agent that modulates a HERPUD1 function, comprising (a) providing an agent that modulates POSH and (b) testing the effect of the agent on a HERPUD1 function. In certain embodiments, testing the effect of the agent on a HERPUD1 function comprises contacting a cell with the agent and measuring the effect of the agent on ubiquitination of HERPUD1.

In certain aspects, the application relates to a method of inhibiting an activity of a POSH-AP in a cell, comprising contacting the cell with an inhibitor of POSH. In certain preferred embodiments, the POSH-AP is HERPUD1.

The application further relates to a method of identifying a modulator of POSH, comprising (a) forming a mixture comprising a POSH polypeptide, a POSH-AP, ubiquitin and a test agent and (b) detecting ubiquitination of the POSH-AP, wherein an agent that inhibits ubiquitination of the POSH-AP is an agent that modulates POSH. In certain embodiments, the POSH-AP is HERPUD1.

The application additionally relates to a method of identifying a modulator of POSH, comprising (a) forming a mixture comprising a POSH polypeptide, a POSH-AP, ubiquitin and a test agent and (b) detecting ubiquitination of the POSH-AP, wherein an agent that inhibits ubiquitination of the POSH-AP is an agent that modulates POSH, the method further comprising testing the effect of the agent on POSH-mediated ubiquitination of a second substrate. In certain embodiments, the second substrate is POSH.

In certain further embodiments, the application relates to a method of identifying an agent that inhibits a neurological disorder, comprising (a) forming a mixture comprising a POSH polypeptide, a POSH-AP, ubiquitin and a test agent and (b) detecting ubiquitination of the POSH-AP, wherein an agent that inhibits ubiquitination of the POSH-AP is an agent that inhibits a neurological disorder. In certain embodiments, the POSH-AP is HERPUD1.

The application further relates to a method of identifying an agent that inhibits a neurological disorder, comprising (a) forming a mixture comprising a POSH polypeptide, a POSH-AP, ubiquitin and a test agent and (b) detecting ubiquitination of the POSH-AP, wherein an agent that inhibits ubiquitination of the POSH-AP is an agent that inhibits a neurological disorder, further comprising testing the effect of the agent on POSH-mediated ubiquitination of a second substrate. In certain further embodiments, the second substrate is POSH.

The present application futher relates to a method of treating a neurological disorder comprising administering an agent to a subject in need thereof, wherein said agent inhibits a ubiquitin ligase activity of POSH. In certain embodiments, the agent inhibits POSH-mediated ubiquitination of HERPUD1. In further embodiments, the agent does not substantially inhibit POSH auto-ubiquitination. In certain embodiments, the application relates to a method of treating a neurological disorder comprising administering an agent to a subject in need thereof, wherein said agent inhibits the ubiquitination of a POSH-AP. In certain embodiments, the POSH-AP is HERPUD1. In further embodiments, the agent does not substantially inhibit POSH auto-ubiquitination.

In certain embodiments, an agent is selected from among: an siRNA construct, a small molecule, an antibody, and an antisense construct.

Examples of small molecules include:

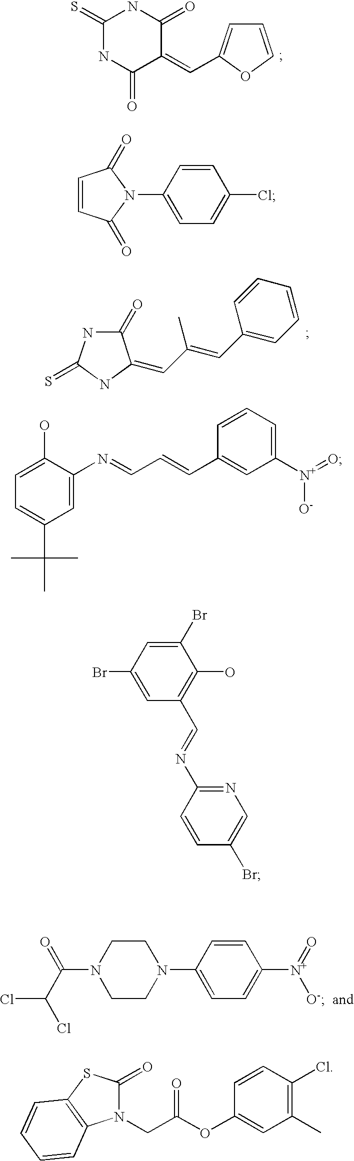

The application further relates to a method of inhibiting the progression of a neurological disorder, comprising administering an agent to a subject in need thereof, wherein said agent inhibits the interaction between a POSH polypeptide and a POSH-AP. In preferred embodiments of the application, the POSH-AP is HERPUD1.

In yet other embodiments, the application further provides a method of testing an agent for use in treatment of a neurological disorder, comprising contacting cells that produce amyloid polypeptide with an agent that inhibits POSH activity and/or expression. In certain embodiments, the agent inhibits POSH ubiquitin ligase activity. In certain further embodiments, the agent inhibits POSH-mediated ubiquitination of HERPUD1. In certain embodiments, the agent inhibits the expression of POSH. In certain further embodiments of the application, the agent is selected from among: an siRNA construct, a small molecule, an antibody, and an antisense construct.

In certain embodiments, the application further provides a method of testing an agent for use in treatment of a neurological disorder, comprising contacting cells that produce amyloid polypeptide with an agent that inhibits POSH activity and/or expression, the method further comprising evaluating the effect of the agent on apoptosis in the cell.

The methods and compositions of the subject application can be used to treat or prevent POSH-associated neurological disorders. Examples of POSH-associated neurological disorders include Alzheimer's disease, Parkinson's disease, Huntington's disease, Pick's disease, Niemann-Pick's disease, prion-associated diseases, depression, and schizophrenia. In certain preferred embodiments, the methods and compositions of the present application can be used to treat or prevent Alzheimer's disease.

In further embodiments of the application, the application relates to a method of treating or preventing a POSH-associated neurological disorder in a subject comprising administering an agent that inhibits the expression of and/or an activity of a POSH polypeptide to a subject in need thereof, wherein said agent treats or prevents the POSH-associated neurological disorder. POSH-associated neurological disorders include Alzheimer's disease, Parkinson's disease, Huntington's disease, Pick's disease, Niemann-Pick's disease, prion-associated diseases, depression, and schizophrenia.

The practice of the present application will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the application will be apparent from the following detailed description, and from the claims.

### BRIEF DESCRIPTION OF THE DRAWINGS

FIG. 1 shows human POSH coding sequence (SEQ ID NO:1).

FIG. 2 shows human POSH amino acid sequence (SEQ ID NO:2).

FIG. 3 shows human POSH cDNA sequence (SEQ ID NO:3).

FIG. 4 shows 5′ cDNA fragment of human POSH (public gi:10432611; SEQ ID NO:4).

FIG. 5 shows N terminus protein fragment of hPOSH (public gi:10432612; SEQ ID NO:5).

FIG. 6 shows 3′ mRNA fragment of hPOSH (public gi:7959248; SEQ ID NO:6).

FIG. 7 shows C terminus protein fragment of hPOSH (public gi:7959249; SEQ ID NO:7).

FIG. 8 shows human POSH full mRNA, annotated sequence.

FIG. 9 shows domain analysis of human POSH.

FIG. 10 is a diagram of human POSH nucleic acids. The diagram shows the full-length POSH gene and the position of regions amplified by RT-PCR or targeted by siRNA used in FIG. 11.

FIG. 11 shows effect of knockdown of POSH mRNA by siRNA duplexes. HeLa SS-6 cells were transfected with siRNA against Lamin A/C (lanes 1, 2) or POSH (lanes 3-10). POSH siRNA was directed against the coding region (153—lanes 3, 4; 155—lanes 5, 6) or the 3′UTR (157—lanes 7, 8; 159—lanes 9, 10). Cells were harvested 24 hours post-transfection, RNA extracted, and POSH mRNA levels compared by RT-PCR of a discrete sequence in the coding region of the POSH gene (see FIG. 10). GAPDH is used an RT-PCR control in each reaction.

FIG. 12 shows that POSH affects the release of VLP from cells. A) Phosphohimages of SDS-PAGE gels of immunoprecipitations of 35S pulse-chase labeled Gag proteins are presented for cell and viral lysates from transfected HeLa cells that were either untreated or treated with POSH RNAi (50 nM for 48 hours). The time during the chase period (1, 2, 3, 4, and 5 hours after the pulse) are presented from left to right for each image.

FIG. 13 shows release of VLP from cells at steady state. Hela cells were transfected with an HIV-encoding plasmid and siRNA. Lanes 1, 3 and 4 were transfected with wild-type HIV-encoding plasmid. Lane 2 was transfected with an HIV-encoding plasmid which contains a point mutation in p6 (PTAP to ATAP). Control siRNA (lamin A/C) was transfected to cells in lanes 1 and 2. siRNA to Tsg101 was transfected in lane 4 and siRNA to POSH in lane 3.

FIG. 14 shows mouse POSH mRNA sequence (public gi:10946921; SEQ ID NO: 8).

FIG. 15 shows mouse POSH Protein sequence (Public gi:10946922; SEQ ID NO: 9).

FIG. 16 shows Drosophila melanogaster POSH mRNA sequence (public gi:17737480; SEQ ID NO: 10).

FIG. 17 shows Drosophila melanogaster POSH protein sequence (public gi:17737481; SEQ ID NO: 11).

FIG. 18 shows POSH domain analysis.

FIG. 19 shows that human POSH has ubiquitin ligase activity.

FIG. 20 shows that POSH knockdown results in decreased secretion of phospholipase D (“PLD”).

FIG. 21 shows effect of hPOSH on Gag-EGFP intracellular distribution.

FIG. 22 shows intracellular distribution of HIV-1 Nef in hPOSH-depleted cells.

FIG. 23 shows intracellular distribution of Src in hPOSH-depleted cells.

FIG. 24 shows intracellular distribution of Rapsyn in hPOSH-depleted cells.

FIG. 25 shows that knock-down of human POSH entraps HIV virus particles in intracellular vesicles. HIV virus release was analyzed by electron microscopy following siRNA and full-length HIV plasmid transfection. Mature viruses were secreted by cells transfected with HIV plasmid and non-relevant siRNA (control, bottom panel). Knockdown of Tsg101 protein resulted in a budding defect, the viruses that were released had an immature phenotype (top panel). Knockdown of hPOSH levels resulted in accumulation of viruses inside the cell in intracellular vesicles (middle panel).

FIG. 26 shows that siRNA-mediated reduction in HERPUD1 expression reduces HIV maturation.

FIG. 27 shows that endogenous Herp levels are reduced in H153 cells. H153 (POSH-RNAi) and H187 (control RNAi) cells were transfected with a plasmid encoding Flag-ubiquitin. Total cell lysates (A) or Flag-immunoprecipitated material (B) were separated on 10% SDS-PAGE and immunoblotted with anti-Herp antibodies.

FIG. 28 shows that exogenous Herp levels and its ubiquitination are reduced in POSH-depleted cells. H153 and H187 cells were co-transfected with Herp or control plasmids and a plasmid encoding Flag-ubiquitin (indicated above the figure). Total (A) and flag-immunoprecipitated material (B) were separated on 10% SDS-PAGE and immunoblotted with anti-Herp antibodies.

FIG. 29 shows that amyloid precursor protein levels are reduced in cells that have reduced levels of POSH. HeLa SS6 cells that express reduced levels of POSH (H153) and control cells expressing scrambled RNAi (H187) were transfected with a plasmid expressing amyloid precursor protein (APP) and presenilin 1 (PS1). Cells were metabolic labeled and protein extracts were immunoprecipitated with anti-amyloid beta specific antibody, which recognize an epitope common to APP, C199 and AP polypeptides. A labeled protein was specifically precipitated by the antibody in H187-transfected cells (see Lanes 3 and 5). However, this polypeptide was not recognized in H153 cells (see Lanes 4 and 6) indicating that APP steady state levels are reduced in H153 and may be rapidly degraded in these cells.

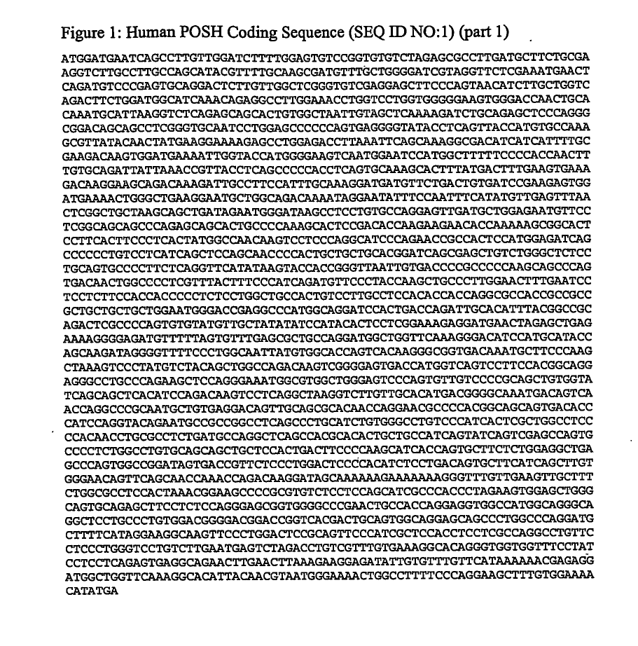

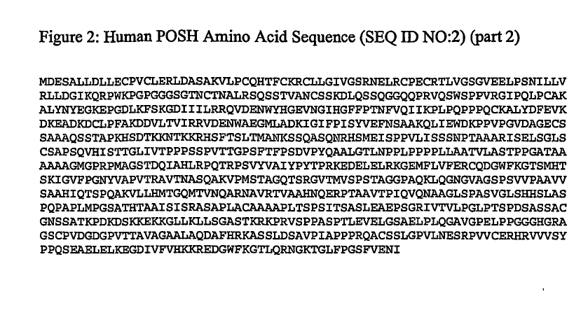

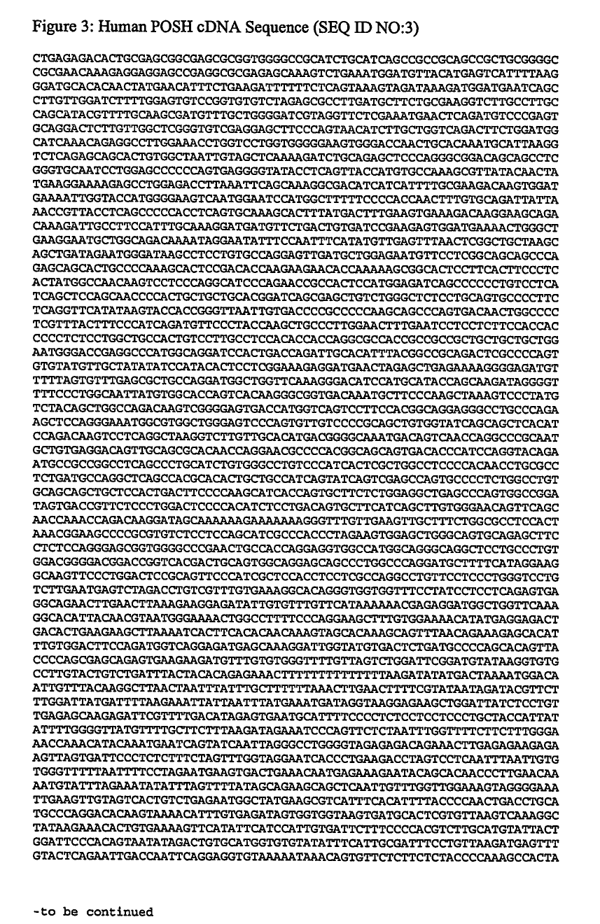

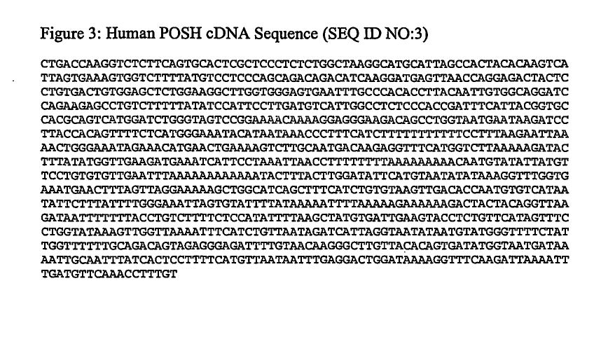

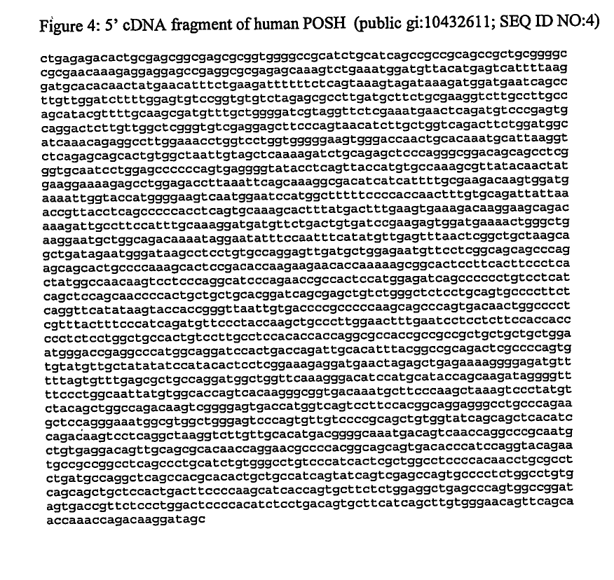

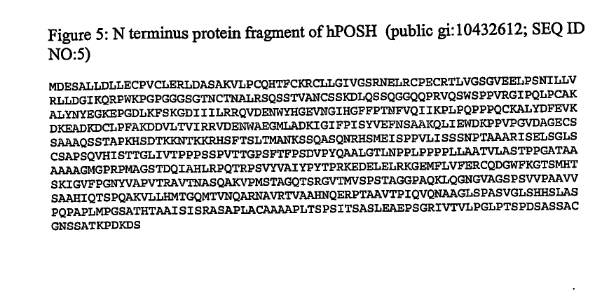

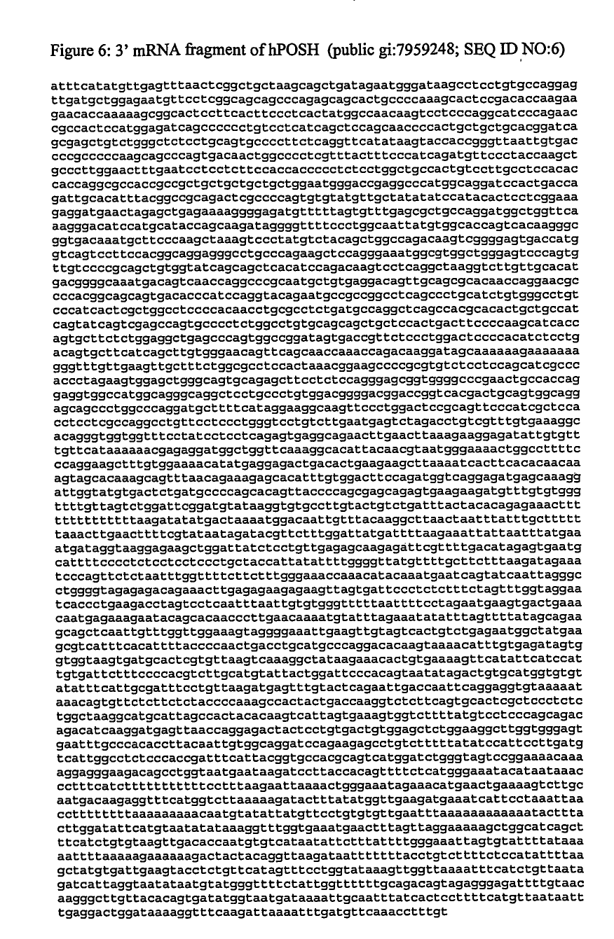

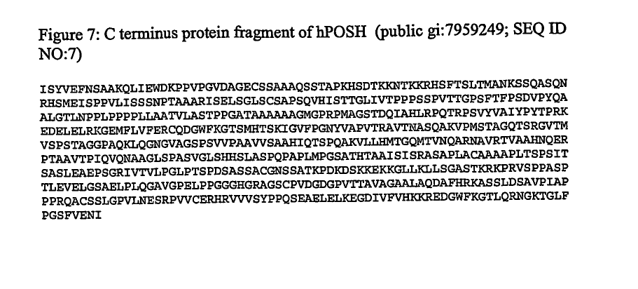

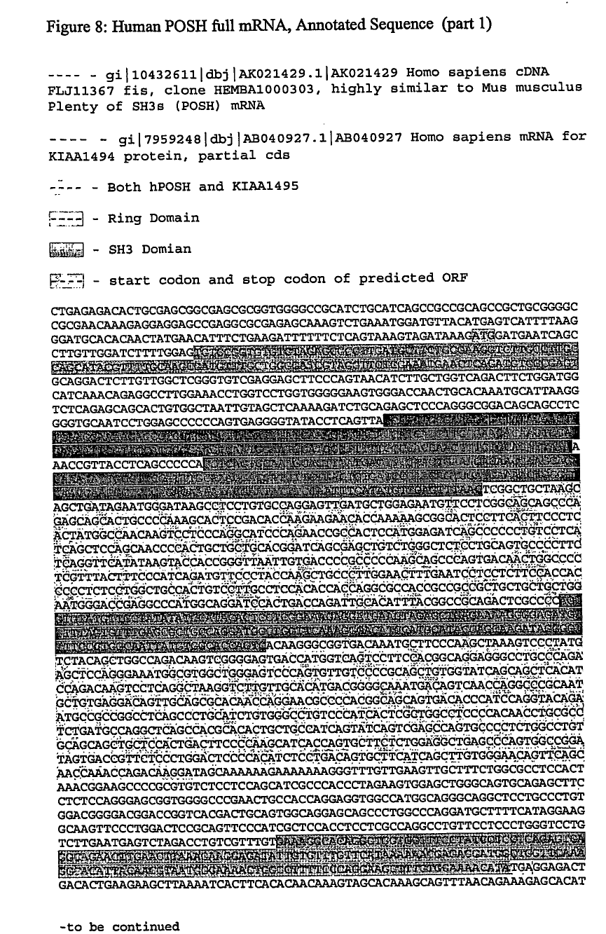

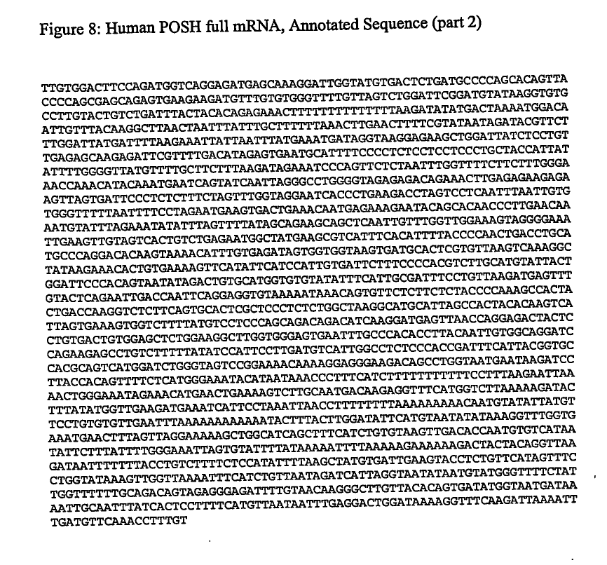

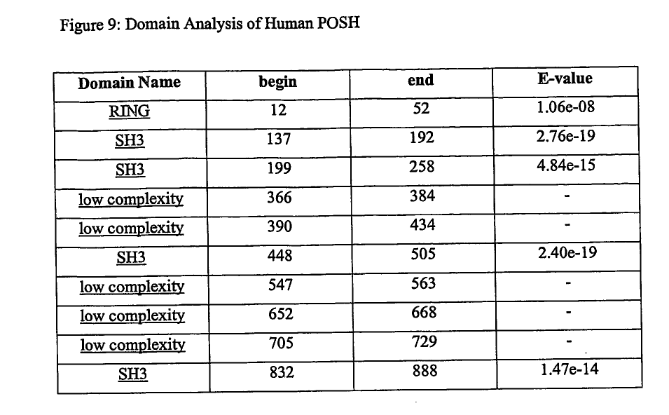

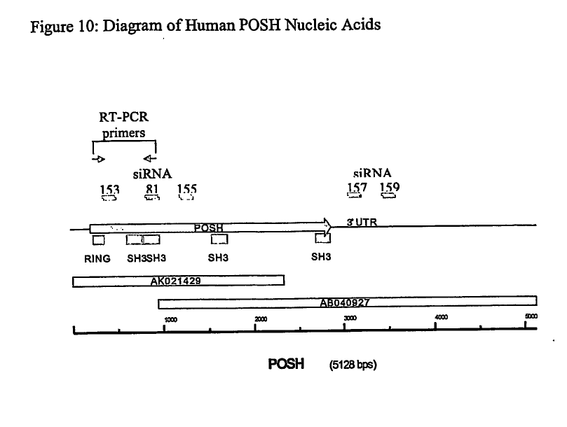

### DETAILED DESCRIPTION OF THE APPLICATION

1. Definitions

The term “amyloid polypeptide” is used to refer to any of the various polypeptides that are significant components of amyloid plaque as well as precursors thereof. The Amyloid beta A4 precursor protein (“APP”) gives rise to smaller proteins, such as the roughly 40 amino acid beta amyloid proteins that form a major component of the amyloid plaque associated with Alzheimer's disease, Down's syndrome (in older patients) and certain hereditary cerebral hemorrhage amyloidoses. APP has several isoforms generated by alternative splicing of a 19-exon gene: exons 1-13, 13a, and 14-18 (Yoshikai et al., 1990). The predominant transcripts are APP695 (exons 1-6, 9-18, not 13a), APP751 (exons 1-7, 9-18, not 13a), and APP770 (exons 1-18, not 13a). All of these encode multidomain proteins with a single membrane-spanning region. They differ in that APP751 and APP770 contain exon 7, which encodes a serine protease inhibitor domain. APP695 is a predominant form in neuronal tissue, whereas APP751 is the predominant variant elsewhere. Beta-amyloid is derived from that part of the protein encoded by parts of exons 16 and 17. All of the isoforms of APP and any of the smaller proteins derived therefrom are included in the term “amyloid polypeptide”, as well as any of the various naturally occuring variations thereof and any artificially produced variants that retain one or more functional properties of the naturally occuring protein or that are useful as a proxy for monitoring the production of APP or a protein derived therefrom. The subset of amyloid polypeptides that are APP or derived therefrom may be referred to specifically as “APP amyloid polypeptides”. Yoshikai et al. Gene 87: 257-263, 1990.

The term “binding” refers to a direct association between two molecules, due to, for example, covalent, electrostatic, hydrophobic, ionic and/or hydrogen-bond interactions under physiological conditions.

A “chimeric protein” or “fusion protein” is a fusion of a first amino acid sequence encoding a polypeptide with a second amino acid sequence defining a domain foreign to and not substantially homologous with any domain of the first amino acid sequence. A chimeric protein may present a foreign domain which is found (albeit in a different protein) in an organism which also expresses the first protein, or it may be an “interspecies”, “intergenic”, etc. fusion of protein structures expressed by different kinds of organisms.

The terms “compound”, “test compound” and “molecule” are used herein interchangeably and are meant to include, but are not limited to, peptides, nucleic acids, carbohydrates, small organic molecules, natural product extract libraries, and any other molecules (including, but not limited to, chemicals, metals and organometallic compounds).

The phrase “conservative amino acid substitution” refers to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). Examples of amino acid groups defined in this manner include:
(i) a charged group, consisting of Glu and Asp, Lys, Arg and His,
(ii) a positively-charged group, consisting of Lys, Arg and His,
(iii) a negatively-charged group, consisting of Glu and Asp,
(iv) an aromatic group, consisting of Phe, Tyr and Trp,
(v) a nitrogen ring group, consisting of His and Trp,
(vi) a large aliphatic nonpolar group, consisting of Val, Leu and Ile,
(vii) a slightly-polar group, consisting of Met and Cys,
(viii) a small-residue group, consisting of Ser, Thr, Asp, Asn, Gly, Ala, Glu, Gln and Pro,
(ix) an aliphatic group consisting of Val, Leu, Ile, Met and Cys, and
(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

A “conserved residue” is an amino acid that is relatively invariant across a range of similar proteins. Often conserved residues will vary only by being replaced with a similar amino acid, as described above for “conservative amino acid substitution”.

The term “domain” as used herein refers to a region of a protein that comprises a particular structure and/or performs a particular function.

The term “envelope virus” as used herein refers to any virus that uses cellular membrane and/or any organelle membrane in the viral release process.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present application. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. The nucleic acid and protein sequences of the present application may be used as a “query sequence” to perform a search against public databases to, for example, identify other family members, related sequences or homologs. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to nucleic acid molecules of the application. BLAST protein searches can be performed with the XBLAST program, score=50, wordlength=3 to obtain amino acid sequences homologous to protein molecules of the application. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used. See http://www.ncbi.nlm.nih.gov.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

The term “isolated”, as used herein with reference to the subject proteins and protein complexes, refers to a preparation of protein or protein complex that is essentially free from contaminating proteins that normally would be present with the protein or complex, e.g., in the cellular milieu in which the protein or complex is found endogenously. Thus, an isolated protein complex is isolated from cellular components that normally would “contaminate” or interfere with the study of the complex in isolation, for instance while screening for modulators thereof. It is to be understood, however, that such an “isolated” complex may incorporate other proteins the modulation of which, by the subject protein or protein complex, is being investigated.

The term “isolated” as also used herein with respect to nucleic acids, such as DNA or RNA, refers to molecules in a form which does not occur in nature. Moreover, an “isolated nucleic acid” is meant to include nucleic acid fragments which are not naturally occurring as fragments and would not be found in the natural state.

Lentiviruses include primate lentiviruses, e.g., human immunodeficiency virus types 1 and 2 (HIV-1/HIV-2); simian immunodeficiency virus (SIV) from Chimpanzee (SIVcpz), Sooty mangabey (SIVsmm), African Green Monkey (SIVagm), Syke's monkey (SIVsyk), Mandrill (SIVmnd) and Macaque (SIVmac). Lentiviruses also include feline lentiviruses, e.g., Feline immunodeficiency virus (FIV); Bovine lentiviruses, e.g., Bovine immunodeficiency virus (BIV); Ovine lentiviruses, e.g., Maedi/Visna virus (MVV) and Caprine arthritis encephalitis virus (CAEV); and Equine lentiviruses, e.g., Equine infectious anemia virus (EIAV). All lentiviruses express at least two additional regulatory proteins (Tat, Rev) in addition to Gag, Pol, and Env proteins. Primate lentiviruses produce other accessory proteins including Nef, Vpr, Vpu, Vpx, and Vif. Generally, lentiviruses are the causative agents of a variety of disease, including, in addition to immunodeficiency, neurological degeneration, and arthritis. Nucleotide sequences of the various lentiviruses can be found in Genbank under the following Accession Nos. (from J. M. Coffin, S. H. Hughes, and H. E. Varmus, “Retroviruses” Cold Spring Harbor Laboratory Press, 199,7 p 804): 1) HIV-1: K03455, M19921, K02013, M3843 1, M38429, K02007 and M17449; 2) HIV-2: M30502, J04542, M30895, J04498, M15390, M31113 and L07625; 3) SIV:M29975, M30931, M58410, M66437, L06042, M33262, M19499, M32741, M31345 and L03295; 4) FIV: M25381, M36968 and U1 1820; 5)BIV. M32690; 6) E1AV: M16575, M87581 and U01866; 6) Visna: M10608, M51543, L06906, M60609 and M60610; 7) CAEV: M33677; and 8) Ovine lentivirus M31646 and M34193. Lentiviral DNA can also be obtained from the American Type Culture Collection (ATCC). For example, feline immunodeficiency virus is available under ATCC Designation No. VR-2333 and VR-3112. Equine infectious anemia virus A is available under ATCC Designation No. VR-778. Caprine arthritis-encephalitis virus is available under ATCC Designation No. VR-905. Visna virus is available under ATCC Designation No. VR-779.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

The term “maturation” as used herein refers to the production, post-translational processing, assembly and/or release of proteins that form a viral particle. Accordingly, this includes the processing of viral proteins leading to the pinching off of nascent virion from the cell membrane.

A “POSH nucleic acid” is a nucleic acid comprising a sequence as represented in any of SEQ ID NOs: 1, 3, 4, 6, 8, and 10 as well as any of the variants described herein.

A “POSH polypeptide” or “POSH protein” is a polypeptide comprising a sequence as represented in any of SEQ ID NOs: 2, 5, 7, 9 and 11 as well as any of the variations described herein.

A “POSH-associated protein” or “POSH-AP” refers to a protein capable of interacting with and/or binding to a POSH polypeptide. Generally, the POSH-AP may interact directly or indirectly with the POSH polypeptide. A preferred POSH-AP of the application is HERPUD1. Examples of HERPUD1 polypeptides are provided throughout.

The terms peptides, proteins and polypeptides are used interchangeably herein.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry, weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 85% by weight, more preferably 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

A “recombinant nucleic acid” is any nucleic acid that has been placed adjacent to another nucleic acid by recombinant DNA techniques. A “recombined nucleic acid” also includes any nucleic acid that has been placed next to a second nucleic acid by a laboratory genetic technique such as, for example, tranformation and integration, transposon hopping or viral insertion. In general, a recombined nucleic acid is not naturally located adjacent to the second nucleic acid.

The term “recombinant protein” refers to a protein of the present application which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

A “RING domain” or “Ring Finger” is a zinc-binding domain with a defined octet of cysteine and histidine residues. Certain RING domains comprise the consensus sequences as set forth below (amino acid nomenclature is as set forth in Table 1): Cys Xaa Xaa Cys Xaa₁₀₋₂₀ Cys Xaa His Xaa₂₋₅ Cys Xaa Xaa Cys Xaa₁₃₋₅₀ Cys Xaa Xaa Cys or Cys Xaa Xaa Cys Xaa₁₀₋₂₀ Cys Xaa His Xaa₂₋₅ His Xaa Xaa Cys Xaa₁₃₋₅₀ Cys Xaa Xaa Cys. Certain RING domains are represented as amino acid sequences that are at least 80% identical to amino acids 12-52 of SEQ ID NO: 2 and is set forth in SEQ ID No: 26. Preferred RING domains are 85%, 90%, 95%, 98% and, most preferably, 100% identical to the amino acid sequence of SEQ ID NO: 26. Preferred RING domains of the application bind to various protein partners to form a complex that has ubiquitin ligase activity. RING domains preferably interact with at least one of the following protein types: F box proteins, E2 ubiquitin conjugating enzymes and cullins.

The term “RNA interference” or “RNAi” refers to any method by which expression of a gene or gene product is decreased by introducing into a target cell one or more double-stranded RNAs which are homologous to the gene of interest (particularly to the messenger RNA of the gene of interest). RNAi may also be achieved by introduction of a DNA:RNA hybrid wherein the antisense strand (relative to the target) is RNA. Either strand may include one or more modifications to the base or sugar-phosphate backbone. Any nucleic acid preparation designed to achieve an RNA interference effect is referred to herein as an siRNA construct Phosphorothioate is a particularly common modification to the backbone of an siRNA construct. siRNA constructs include short hairpin RNA (shRNA) constructs.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the application.

An “SH3” or “Src Homology 3” domain is a protein domain of generally about 60 amino acid residues first identified as a conserved sequence in the non-catalytic part of several cytoplasmic protein tyrosine kinases (e.g., Src, Abl, Lck).

SH3 domains mediate assembly of specific protein complexes via binding to proline-rich peptides. Exemplary SH3 domains are represented by amino acids 137-192, 199-258, 448-505 and 832-888 of SEQ ID NO:2 and are set forth in SEQ ID Nos: 27-30. In certain embodiments, an SH3 domain interacts with a consensus sequence of RXaaXaaPXaaX6P (where X6, as defined in table 1 below, is a hydrophobic amino acid). In certain embodiments, an SH3 domain interacts with one or more of the following sequences: P(T/S)AP, PFRDY, RPEPTAP, RQGPKEP, RQGPKEPFR, RPEPTAPEE and RPLPVAP.

As used herein, the term “specifically hybridizes” refers to the ability of a nucleic acid probe/primer of the application to hybridize to at least 12, 15, 20, 25, 30, 35, 40, 45, 50 or 100 consecutive nucleotides of a POSH sequence, or a sequence complementary thereto, or naturally occurring mutants thereof, such that it has less than 15%, preferably less than 10%, and more preferably less than 5% background hybridization to a cellular nucleic acid (e.g., mRNA or genomic DNA) other than the POSH gene. A variety of hybridization conditions may be used to detect specific hybridization, and the stringency is determined primarily by the wash stage of the hybridization assay. Generally high temperatures and low salt concentrations give high stringency, while low temperatures and high salt concentrations give low stringency. Low stringency hybridization is achieved by washing in, for example, about 2.0×SSC at 50° C., and high stringency is acheived with about 0.2×SSC at 50° C. Further descriptions of stringency are provided below.

As applied to polypeptides, “substantial sequence identity” means that two peptide sequences, when optimally aligned, such as by the programs GAP or BESTFIT using default gap which share at least 90 percent sequence identity, preferably at least 95 percent sequence identity, more preferably at least 99 percent sequence identity or more. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

As is well known, genes for a particular polypeptide may exist in single or multiple copies within the genome of an individual. Such duplicate genes may be identical or may have certain modifications, including nucleotide substitutions, additions or deletions, which all still code for polypeptides having substantially the same activity.

A “virion” is a complete viral particle; nucleic acid and capsid (and a lipid envelope in some viruses. A “viral particle” may be incomplete, as when produced by a cell transfected with a defective virus (e.g., an HIV virus-like particle system).

TABLE 1
Abbreviations for classes of amino acids*

| | | Amino Acids |
|---|---|---|
| Symbol | Category | Represented |
| X1 | Alcohol | Ser, Thr |
| X2 | Aliphatic | Ile, Leu, Val |
| Xaa | Any | Ala, Cys, Asp, Glu, Phe, |
| | | Gly, His, Ile, Lys, Leu, |
| | | Met, Asn, Pro, Gln, Arg, |
| | | Ser, Thr, Val, Trp, Tyr |
| X4 | Aromatic | Phe, His, Trp, Tyr |
| X5 | Charged | Asp, Glu, His, Lys, Arg |
| X6 | Hydrophobic | Ala, Cys, Phe, Gly, His, |
| | | Ile, Lys, Leu, Met, Thr, |
| | | Val, Trp, Tyr |
| X7 | Negative | Asp, Glu |
| X8 | Polar | Cys, Asp, Glu, His, Lys, |
| | | Asn, Gln, Arg, Ser, Thr |
| X9 | Positive | His, Lys, Arg |
| X10 | Small | Ala, Cys, Asp, Gly, Asn, |
| | | Pro, Ser, Thr, Val |
| X11 | Tiny | Ala, Gly, Ser |
| X12 | Turnlike | Ala, Cys, Asp, Glu, Gly, |
| | | His, Lys, Asn, Gln, Arg, |
| | | Ser, Thr |
| X13 | Asparagine-Aspartate | Asn, Asp |

*Abbreviations as adopted from http://smart.embl-heidelberg.de/SMART_DATA/alignments/consensus/grouping.html.

2. Overview

In certain aspects, the application relates to the discovery of novel associations between POSH proteins and other proteins (termed POSH-APs), and related methods and compositions. In certain aspects, the application relates to novel associations among certain disease states, POSH nucleic acids and proteins, and POSH-AP nucleic acids and proteins. In preferred embodiments, the application relates to the discovery of novel associations between POSH proteins and HERPUD1 proteins, and related methods and compositions. In further embodiments, the application relates to novel associations among certain disease states, POSH nucleic acids and proteins, and HERPUD1 nucleic acids and proteins.

In certain aspects, by identifying proteins associated with POSH, and particularly human POSH, the present application provides a conceptual link between the POSH-APs and cellular processes and disorders associated with POSH-APs, and POSH itself. Accordingly, in certain embodiments of the disclosure, agents that modulate a POSH-AP, such as HEPRUD1, may now be used to modulate POSH functions and disorders associated with POSH function, such as neurological disorders. Additionally, test agents may be screened for an effect on a POSH-AP, such as HERPUD1, and then further tested for an effect on a POSH function or a disorder associated with POSH function. Likewise, in certain embodiments of the disclosure, agents that modulate POSH may now be used to modulate POSH-AP, such as HERPUD1, functions and disorders associated with POSH-AP function, such as disorders associated with HERPUD1 function, including HERPUD1-associated neurological disorders. Additionally, test agents may be screened for an effect on HERPUD1 and then further tested for effect on a POSH-AP function or a disorder associated with POSH-AP function. In further aspects, the application provides nucleic acid agents (e.g., RNAi probes, antisense nucleic acids), antibody-related agents, small molecules and other agents that affect POSH function, and the use of same in modulating POSH and/or POSH-AP activity.

In certain aspects, the application relates to the discovery that a POSH polypeptide interacts with one or more HERPUD1 polypeptides. Accordingly, the application provides complexes comprising POSH and HERPUD1. In one aspect, the application relates to the discovery that POSH binds directly with HERPUD1. This interaction was identified by Applicants in a yeast 2-hybrid assay. HERPUD1 is synonymous with Herp, and the terms are used interchangeably herein.

In certain aspects, the application relates to the discovery that a POSH polypeptide interacts with HERPUD1, a “homocysteine-inducible, endoplasmic reticulum stress-inducible, ubiquitin-like domain member 1” protein. In part, the present application relates to the discovery that the POSH-AP, HERPUD1, is involved in the maturation of an envelope virus, such as HIV.

Certain HERPUD1 polypeptides are involved in JNK-mediated apoptosis, particularly in vascular endothelial cells, including cells that are exposed to high levels of homocysteine. Certain HERPUD1 polypeptides are involved in the Unfolded Protein Response, a cellular response to the presence of unfolded proteins in the endoplasmic reticulum. Certain HERPUD1 polypeptides are involved in the regulation of sterol biosynthesis. Accordingly, certain POSH polypeptides are involved in the Unfolded Protein Response and sterol biosynthesis.

In other aspects, certain HERPUD1 polypeptides enhance presenilin-mediated amyloid beta-protein generation. For example, HERPUD1 polypeptides, when overexpressed in cells, increase the level of amyloid beta generation, and it has been observed that HERPUD1 polypeptides interact with the presenilin proteins, presenilin-1 and presenilin-2. (See Sai, X. et al (2002) J. Biol. Chem. 277:12915-12920). Accordingly, in certain aspects, POSH polypeptides may modulate the level of amyloid beta generation. Additionally, POSH polypeptides may interact with presenilin 1 and presenilin 2. Therefore, it is believed certain POSH polypeptides modulate presenilin-mediated amyloid beta generation. The accumulation of amyloid beta is one hallmark of Alzheimer's disease. Accordingly, these POSH polypeptides may be involved in the pathogenesis of Alzheimer's disease. At sites such as late intracellular compartment sites including the trans-Golgi network, certain mutant presenilin-2 polypeptides up-regulate production of amyloid beta peptides ending at position 42 (Aβ42). (See Iwata, H. et al (2001) J. Biol. Chem. 276: 21678-21685). Accordingly, POSH polypeptides may regulate production of Aβ42 through mutant presenilin-2 at late intracellular compartment sites including the trans-Golgi network. Furthermore, elevated homocysteine levels have been found to be a risk factor associated with Alzheimer's disease and cerebral vascular disease.

Some risk factors, such as elevated plasma homocysteine levels, may accelerate or increase the severity of several central nervous system (CNS) disorders. Elevated levels of plasma homocysteine were found in young male patients with schizophrenia suggesting that elevated homocysteine levels could be related to the pathophysiology of aspects of schizophrenia (Levine, J. et al (2002) Am. J. Psychiatry 159:1790-2). Epidemiological and experimental studies have linked increased homocysteine levels with neurodegenerative conditions, including Alzheimer's disease, Parkinson's disease, depression, and stroke (reviewed in Mattson, M P and Shea, T B (2003) Trends Neurosci 26:137-46).

Accordingly, certain POSH polypeptides may be involved in neurological disorders. Neurological disorders include disorders associated with increased levels of plasma homocysteine, increased levels of amyloid beta production, or aberrant presenilin acitivity. Neurological disorders include CNS disorders, such as Alzheimer's disease, cerebral vascular disease, and schizophrenia.

Certain POSH polypeptides may be involved in cardiovascular diseases, such as thromboembolic vascular disease, and particularly the disease characteristics associated with hyperhomocysteinemia. See, for example, Kokame et al. 2000 J. Biol. Chem. 275:32846-53; Zhang et al. 2001 Biochem Biophys Res Commun 289:718-24.

The term HERPUD1 is used herein to refer as well to various naturally occurring HERPUD1 homologs, as well as functionally similar variants and fragments that retain at least 80%, 90%, 95%, or 99% sequence identity to a naturally occurring HERPUD1. The term specifically includes human HERPUD1 nucleic acid and amino acid sequences and the sequences presented in the Examples.

Examples of additional POSH-APs include PTPN12, DDEF1, EPS8L2, GOCAP, CBL-B, SIAH1, SMN1, SMN2, TTC3, SPG20, SNX1, and ARF1.

POSH intersects with and regulates a wide range of key cellular functions that may be manipulated by affecting the level of and/or activity of POSH polypeptides or POSH-AP polypeptides. Many features of POSH, and particularly human POSH, are described in PCT patent publications WO03/095971A2 (application no. WO2002US0036366) and WO03/078601A2 (application, no. WO2003US0008194) the teachings of which are incorporated by reference herein.

As described in the above-referenced publications, native human POSH is a large polypeptide containing a RING domain and four SH3 domains. POSH is a ubiquitin ligase (also termed an “E3” enzyme); the RING domain mediates ubiquitination of, for example, the POSH polypeptide itself. POSH interacts with a large number of proteins and participates in a host of different biological processes.

As demonstrated in this disclosure, POSH associates with a number of different proteins in the cell. POSH co-localizes with proteins that are known to be located in the trans-Golgi network, implying that POSH participates in the trafficking of proteins in the secretory system. The term “secretory system” should be understood as referring to the membrane compartments and associated proteins and other molecules that are involved in the movement of proteins from the site of translation to a location within a vacuole, a compartment in the secretory pathway itself, a lysosome or endosome or to a location at the plasma membrane or outside the cell. Commonly cited examples of compartments in the secretory system include the endoplasmic reticulum, the Golgi apparatus and the cis and trans Golgi networks. In addition, Applicants have demonstrated that POSH is necessary for proper secretion, localization or processing of a variety of proteins, including phospholipase D, HIV Gag, HIV Nef, Rapsyn and Src. Many of these proteins are myristoylated, indicating that POSH plays a general role in the processing and proper localization of myristoylated proteins. Accordingly, in certain aspects, POSH may play a role in the processing and proper localization of myristolyated proteins. N-myristoylation is an acylation process, which results in covalent attachment of myristate, a 14-carbon saturated fatty acid to the N-terminal glycine of proteins (Farazi et al., J. Biol. Chem. 276: 39501-04 (2001)). N-myristoylation occurs co-translationaly and promotes weak and reversible protein-membrane interaction. Myristoylated proteins are found both in the cytoplasm and associated with membrane. Membrane association is dependent on protein configuration, i.e., surface accessibility of the myristoyl group may be regulated by protein modifications, such as phosphorylation, ubiquitination etc. Modulation of intracellular transport of myristoylated proteins in the application includes effects on transport and localization of these modified proteins.

As described herein, POSH and HERPUD1 are involved in viral maturation, including the production, post-translational processing, assembly and/or release of proteins in a viral particle. Accordingly, viral infections may be ameliorated by inhibiting an activity of HERPUD1 or POSH (e.g., inhibition of ubiquitin ligase activity), and in preferred embodiments, the virus is a retroid virus, an RNA virus or an envelope virus, including HIV, Ebola, HBV, HCV, HTLV, West Nile Virus (WNV) or Moloney Murine Leukemia Virus (MMuLV). Additional viral species are described in greater detail below. In certain instances, a decrease of a POSH function is lethal to cells infected with a virus that employs POSH in release of viral particles.

In certain aspects, the application describes an hPOSH interaction with Rac, a small GTPase and the POSH associated kinases MLK, MKK and JNK. Rho, Rac and Cdc42 operate together to regulate organization of the actin cytoskeleton and the MLK-MKK-JNK MAP kinase pathway (referred to herein as the “JNK pathway” or “Rac-JNK pathway” (Xu et al., 2003, EMBO J. 2: 252-61). Ectopic expression of mouse POSH (“mPOSH”) activates the JNK pathway and causes nuclear localization of NF-κB. Overexpression of mPOSH in fibroblasts stimulates apoptosis. (Tapon et al. (1998) EMBO J. 17:1395404). In Drosophila, POSH may interact with, or otherwise influence the signaling of, another GTPase, Ras. (Schnorr et al. (2001) Genetics 159: 609-22). The JNK pathway and NF-κB regulate a variety of key genes involved in, for example, immune responses, inflammation, cell proliferation and apoptosis. For example, NF-κB regulates the production of interleukin 1, interleukin 8, tumor necrosis factor and many cell adhesion molecules. NF-κB has both pro-apoptotic and anti-apoptotic roles in the cell (e.g., in FAS-induced cell death and TNF-alpha signaling, respectively). NF-κB is negatively regulated, in part, by the inhibitor proteins IκBα and IκBβ (collectively termed “IκB”). Phosphorylation of IκB permits activation and nuclear localization of NF-κB. Phosphorylation of IκB triggers its degradation by the ubiquitin system. In an additional embodiment, a POSH polypeptide promotes nuclear localization of NF-κB. By downregulating POSH, apoptosis may be diminished in certain cells, and this will generally be desirable in conditions characterized by excessive cell death, such as myocardial infarction, stroke, degenerative diseases of muscle and nerve (particularly Alzheimer's disease), and for organ preservation prior to transplant. In a further embodiment, a POSH polypeptide associates with a vesicular trafficking complex, such as a clathrin- or coatomer-containing complex, and particularly a trafficking complex that localizes to the nucleus and/or Golgi apparatus.

As described in WO003/095971A2 (application no. WO2002US0036366) and WO03/078601A2 (application no. WO2003US0008194), POSH polypeptides function as E3 enzymes in the ubiquitination system. Accordingly, downregulation or upregulation of POSH ubiquitin ligase activity can be used to manipulate biological processes that are affected by protein ubiquitination. Modulation of POSH ubiquitin ligase activity may be used to affect POSH and related biological processes, and likewise, modulation of POSH may be used to affect POSH ubiquitin ligase activity and related processes. Downregulation or upregulation may be achieved at any stage of POSH formation and regulation, including transcriptional, translational or post-translational regulation. For example, POSH transcript levels may be decreased by RNAi targeted at a POSH gene sequence. As another example, POSH ubiquitin ligase activity may be inhibited by contacting POSH with an antibody that binds to and interferes with a POSH RING domain or a domain of POSH that mediates interaction with a target protein (a protein that is ubiquitinated at least in part because of POSH activity). As a further example, small molecule inhibitors of POSH ubiquitin ligase activity are provided herein. As another example, POSH activity may be increased by causing increased expression of POSH or an active portion thereof. POSH, and POSH-APs that modulate POSH ubiquitin ligase activity may participate in biological processes including, for example, one or more of the various stages of a viral lifecycle, such as viral entry into a cell, production of viral proteins, assembly of viral proteins and release of viral particles from the cell. POSH may participate in diseases characterized by the accumulation of ubiquitinated proteins, such as dementias (e.g., Alzheimer's and Pick's), inclusion body myositis and myopathies, polyglucosan body myopathy, and certain forms of amyotrophic lateral sclerosis. POSH may participate in diseases characterized by excessive or inappropriate ubiquitination and/or protein degradation.

4. Methods and Compositions for Treating POSH and POSH-AP-Associated Diseases

In certain aspects, the application provides methods and compositions for treatment of POSH-associated diseases (disorders), including neurological disorders. In certain aspects, the application provides methods and compositions for treatment of POSH-AP-associated diseases (disorders), such as HERPUD1-associated disorders, including neurological and viral disorders, as well as neurological disorders associated with unwanted apoptosis, including, for example a variety of neurodegenerative disorders, such as Alzheimer's disease.

Preferred therapeutics of the application for the treatment of a neurological disorder can function by disrupting the biological activity of a POSH polypeptide or POSH complex associated with a neurological disorder. Certain therapeutics of the application function by disrupting the activity of POSH by inhibiting the ubiquitin ligase activity of a POSH polypeptide, such as, for example, by inhibiting the POSH-mediated ubiquitination of HERPUD1.

In certain embodiments, the application relates to methods of treating or preventing neurological disorders. In certain aspects, the invention provides methods and compositions for the identification of compositions that interfere with the function of a POSH or a POSH-AP, such as HERPUD1, which function may relate to aberrant protein processing associated with a neurodegenerative disorder, such as for example, the processing of amyloid beta precursor protein associated with Alzheimer's disease. Neurological disorders include disorders associated with increased levels of amyloid polypeptides, such as for example, Alzheimer's disease. Neurological disorders also include Parkinson's disease, Huntington's disease, schizophrenia, Pick's disease, Niemann-Pick's disease, prion-associated diseases (e.g., Mad Cow disease), depression, and schizophrenia.

Exemplary therapeutics of the application include nucleic acid therapies such as, for example, RNAi constructs (small inhibitory RNAs), antisense oligonucleotides, ribozyme, and DNA enzymes. Other therapeutics include polypeptides, peptidomimetics, antibodies and small molecules.

Antisense therapies of the application include methods of introducing antisense nucleic acids to disrupt the expression of POSH polypeptides or proteins that are necessary for POSH function. Antisense therapies of the application also include methods of introducing antisense nucleic acids to disrupt the expression of POSH-AP polypeptides, such as HERPUD 1 polypeptides, or proteins that are necessary for POSH function.

RNAi therapies include methods of introducing RNAi constructs to downregulate the expression of POSH polypeptides or HERPUD1 polypeptides. Exemplary RNAi therapeutics also include any one of SEQ ID NOs: 15, 16, 18, 19, 21, 22, 24 and 25.

Therapeutic polypeptides may be generated by designing polypeptides to mimic certain protein domains important in the formation of POSH: POSH-AP complexes (e.g., POSH:HERPUD1 complexes), such as, for example, SH3 or RING domains. For example, a polypeptide comprising a POSH SH3 domain such as, for example, the SH3 domain as set forth in SEQ ID NO: 30 will compete for binding to a POSH SH3 domain and will therefore act to disrupt binding of a partner protein.

In view of the specification, methods for generating antibodies directed to epitopes of POSH and HERPUD1 are known in the art. Antibodies may be introduced into cells by a variety of methods. One exemplary method comprises generating a nucleic acid encoding a single chain antibody that is capable of disrupting a POSH:HERPUD1 complex. Such a nucleic acid may be conjugated to antibody that binds to receptors on the surface of target cells. It is contemplated that in certain embodiments, the antibody may target viral proteins that are present on the surface of infected cells, and in this way deliver the nucleic acid only to infected cells. Once bound to the target cell surface, the antibody is taken up by endocytosis, and the conjugated nucleic acid is transcribed and translated to produce a single chain antibody that interacts with and disrupts the targeted POSH:HERPUD1 complex. Nucleic acids expressing the desired single chain antibody may also be introduced into cells using a variety of more conventional techniques, such as viral transfection (e.g., using an adenoviral system) or liposome-mediated transfection.

Small molecules of the application may be identified for their ability to modulate the formation of POSH:HERPUD1 complexes.

Certain embodiments of the disclosure relate to use of a small molecule as an inhibitor of POSH.

Examples of such small molecules include the following compounds:

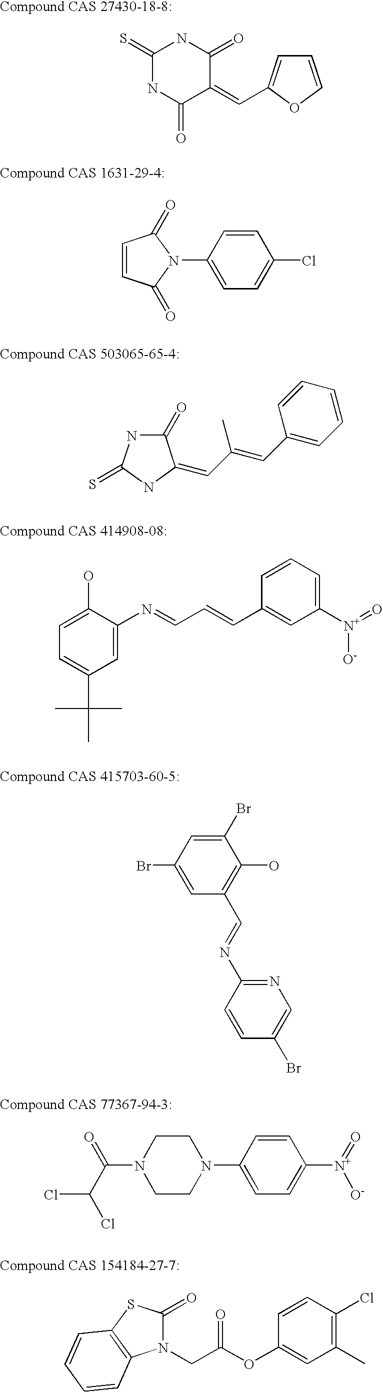

In certain embodiments, compounds useful in the instant compositions and methods include heteroarylmethylene-dihydro-2,4,6-pyrimidinetriones and their thione analogs. Preferred heteroaryl moieties include 5-membered rings such as thienyl, furyl, pyrrolyl, oxazolyl, thiazolyl, and imidazolyl moieties.

In certain embodiments, compounds useful in the instant compositions and methods include N-arylmaleimides, especially N-phenylmaleimides, in which the phenyl group may be substituted or unsubstituted.

In certain embodiments, compounds useful in the instant compositions and methods include arylallylidene-2,4-imidazolidinediones and their thione analogs. Preferred aryl groups are phenyl groups, and both the aryl and allylidene portions of the molecule may be substituted or unsubstituted.

In certain embodiments, compounds useful in the instant compositions and methods include substituted distyryl compounds and aza analogs thereof such as substituted 1,4-diphenylazabutadiene compounds.

In certain other embodiments, compounds useful in the instant compositions and methods include substituted styrenes and aza analogs thereof, such as 1,2-diphenylazaethylenes and 1-phenyl-2-pyridyl-azaethelenes.

In yet other embodiments, compounds useful in the instant compositions and methods include N-aryl-N′-acylpiperazines. In such compounds, the aryl ring, the acyl substituent, and/or the piperazine ring may be substituted or unsubstituted.

In additional embodiments, compounds useful in the instant compositions and methods include aryl esters of (2-oxo-benzooxazol-3-yl)-acetic acid, and analogs thereof in which one or more oxygen atoms are replaced by sulfur atoms.

The generation of nucleic acid based therapeutic agents directed to POSH and POSH-APs, such as HERPUD1, is described below.

Methods for identifying and evaluating further modulators of POSH and POSH-APs, such as HERPUD1, are also provided below.

5. RNA Interference, Ribozymes, Antisense and Related Constructs

In certain aspects, the application relates to RNAi, ribozyme, antisense and other nucleic acid-related methods and compositions for manipulating (typically decreasing) a POSH activity. Specific instances of nucleic acids that may be used to design nucleic acids for RNAi, ribozyme, antisense are provided in the Examples. In certain aspects, the application relates to RNAi, ribozyme, antisense and other nucleic acid-related methods and compositions for manipulating (typically decreasing) a POSH-AP (e.g., HERPUD1) activity. Exemplary RNAi and ribozyme molecules may comprise a sequence as shown in any of SEQ ID NOs: 15, 16, 18, 19, 21, 22, 24 and 25.

Certain embodiments of the application make use of materials and methods for effecting knockdown of one or more POSH or POSH-AP (e.g., HERPUD1) genes by means of RNA interference (RNAi). RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Furthermore, Accordingly, RNAi may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length. The nonspecific effects occur because dsRNA activates two enzymes: PKR, which in its active form phosphorylates the translation initiation factor eIF2 to shut down all protein synthesis, and 2′,5′ oligoadenylate synthetase (2′,5′-AS), which synthesizes a molecule that activates Rnase L, a nonspecific enzyme that targets all mRNAs. The nonspecific pathway may represent a host response to stress or viral infection, and, in general, the effects of the nonspecific pathway are preferably minimized under preferred methods of the present application. Significantly, longer dsRNAs appear to be required to induce the nonspecific pathway and, accordingly, dsRNAs shorter than about 30 bases pairs are preferred to effect gene repression by RNAi (see Hunter et al. (1975) J Biol Chem 250: 409-17; Manche et al. (1992) Mol Cell Biol 12: 523948; Minks et al. (1979) J Biol Chem 254: 10180-3; and Elbashir et al. (2001) Nature 411: 494-8).

RNAi has been shown to be effective in reducing or eliminating the expression of genes in a number of different organisms including Caenorhabditiis elegans (see e.g., Fire et al. (1998) Nature 391: 806-11), mouse eggs and embryos (Wianny et al. (2000) Nature Cell Biol 2: 70-5; Svoboda et al. (2000) Development 127: 4147-56), and cultured RAT-1 fibroblasts (Bahramina et al. (1999) Mol Cell Biol 19: 274-83), and appears to be an anciently evolved pathway available in eukaryotic plants and animals (Sharp (2001) Genes Dev. 15: 485-90). RNAi has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass (2001) Nature 411: 428-9). In mammalian cells, siRNAs are effective at concentrations that are several orders of magnitude below the concentrations typically used in antisense experiments (Elbashir et al. (2001) Nature 411: 494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides of the application may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashir et al. (2001) Nature 411: 494-8). Longer dsRNAs of 50, 75, 100 or even 500 base pairs or more may also be utilized in certain embodiments of the application. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g., Expedite RNA phophoramidites and thymidine phosphoramidite (Proligo, Germany). Synthetic oligonucleotides are preferably deprotected and gel-purified using methods known in the art (see e.g., Elbashir et al. (2001) Genes Dev. 15: 188-200). Longer RNAs may be transcribed from promoters, such as 17 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a POSH or POSH-AP, such as POSH, nucleic acid, such as, for example, a nucleic acid that hybridizes, under stringent and/or physiological conditions, to any of the POSH sequences presented in the Examples, such as, for example, the sequences depicted in SEQ ID NOs: 1, 3, 4, 6, 8 and 10 and complements thereof.

The specific sequence utilized in design of the oligonucleotides may be any contiguous sequence of nucleotides contained within the expressed gene message of the target Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference. Messenger RNA (mRNA) is generally thought of as a linear molecule which contains the information for directing protein synthesis within the sequence of ribonucleotides, however studies have revealed a number of secondary and tertiary structures that exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g., Jaeger et al. (1989) Proc. Natl. Acad. Sci. USA 86:7706 (1989); and Turner et al. (1988) Annu. Rev. Biophys. Biophys. Chem. 17:167). The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerheadribozyme compositions of the application.

The dsRNA oligonucleotides may be introduced into the cell by transfection with an heterologous target gene using carrier compositions such as liposomes, which are known in the art—e.g., Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al. (1998) J Cell Biol 141: 863-74). The effectiveness of the RNAi may be assessed by any of a number of assays following introduction of the dsRNAs. These include Western blot analysis using antibodies which recognize the POSH or POSH-AP (e.g., HERPUD1) gene product following sufficient time for turnover of the endogenous pool after new protein synthesis is repressed, reverse transcriptase polymerase chain reaction and Northern blot analysis to determine the level of existing POSH or POSH-AP (e.g., HERPUD1) target mRNA.

Further compositions, methods and applications of RNAi technology are provided in U.S. Pat. Nos. 6,278,039, 5,723,750 and 5,244,805, which are incorporated herein by reference.

Ribozyme molecules designed to catalytically cleave POSH or POSH-AP mRNA transcripts can also be used to prevent translation of subject POSH or POSH-AP mRNAs and/or expression of POSH or POSH-AP (see, e.g., PCT International Publication WO90/11364, published Oct. 4, 1990; Sarver et al. (1990) Science 247:1222-1225 and U.S. Pat. No. 5,093,246). Ribozymes are enzymatic RNA molecules capable of catalyzing the specific cleavage of RNA. (For a review, see Rossi (1994) Current Biology 4: 469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to a POSH or POSH-AP mRNA, and the well known catalytic sequence responsible for mRNA cleavage or a functionally equivalent sequence (see, e.g., U.S. Pat. No. 5,093,246, which is incorporated herein by reference in its entirety).

While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach ((1988) Nature 334:585-591; and see PCT Appln. No. WO89/05852, the contents of which are incorporated herein by reference). Hammerhead ribozyme sequences can be embedded in a stable RNA such as a transfer RNA (tRNA) to increase cleavage efficiency in vivo (Perriman et al. (1995) Proc. Natl. Acad. Sci. USA, 92: 6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J). In particular, RNA polymerase III-mediated expression of tRNA fusion ribozymes are well known in the art (see Kawasaki et al. (1998) Nature 393: 284-9; Kuwabara et al. (1998) Nature Biotechnol. 16: 961-5; and Kuwabara et al. (1998) Mol. Cell 2: 617-27; Koseki et al. (1999) J Virol 73: 1868-77; Kuwabara et al. (1999) Proc Natl Acad Sci USA 96: 1886-91; Tanabe et al. (2000) Nature 406: 473-4). There are typically a number of potential hammerhead ribozyme cleavage sites within a given target cDNA sequence. Preferably the ribozyme is engineered so that the cleavage recognition site is located near the 5′ end of the target mRNA—to increase efficiency and minimize the intracellular accumulation of non-functional mRNA transcripts. Furthermore, the use of any cleavage recognition site located in the target sequence encoding different portions of the C-terminal amino acid domains of, for example, long and short forms of target would allow the selective targeting of one or the other form of the target, and thus, have a selective effect on one form of the target gene product.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a POSH or POSH-AP mRNA, such as an mRNA of a sequence represented in any of SEQ ID NOs: 1, 3, 4, 6, 8 or 10. In addition, ribozymes possess highly specific endonbonuclease activity, which autocatalytically cleaves the target sense mRNA. The present application extends to ribozymes which hybridize to a sense mRNA encoding a POSH or POSH-AP gene such as a therapeutic drug target candidate gene, thereby hybridizing to the sense mRNA and cleaving it, such that it is no longer capable of being translated to synthesize a functional polypeptide product.

The ribozymes of the present application also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al. (1984) Science 224:574-578; Zaug, et al. (1986) Science 231:470-475; Zaug, et al. (1986) Nature 324:429-433; published International patent application No. WO88/04300 by University Patents Inc.; Been, et al. (1986) Cell 47:207-216). The Cech-type ribozymes have an eight base pair active site which hybridizes to a target RNA sequence whereafter cleavage of the target RNA takes place. The application encompasses those Cech-type ribozymes which target eight base-pair active site sequences that are present in a target gene or nucleic acid sequence.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme. In this aspect of the application, the gene-targeting portions of the ribozyme or RNAi are substantially the same sequence of at least 5 and preferably 6, 7, 8, 9, 10, 11, 12, 13, 14, 15, 16, 17, 18, 19 or 20 or more contiguous nucleotides of a POSH nucleic acid, such as a nucleic acid of any of SEQ ID NOs: 1, 3, 4, 6, 8, or 10. In a long target RNA chain, significant numbers of target sites are not accessible to the ribozyme because they are hidden within secondary or tertiary structures (Birikh et al. (1997) Eur J Biochem 245: 1-16). To overcome the problem of target RNA accessibility, computer generated predictions of secondary structure are typically used to identify targets that are most likely to be single-stranded or have an “open” configuration (see Jaeger et al. (1989) Methods Enzymol 183: 281-306). Other approaches utilize a systematic approach to predicting secondary structure which involves assessing a huge number of candidate hybridizing oligonucleotides molecules (see Milner et al. (1997) Nat Biotechnol 15: 53741; and Patzel and Sczakiel (1998) Nat Biotechnol 16: 64-8). Additionally, U.S. Pat. No. 6,251,588, the contents of which are hereby incorporated herein, describes methods for evaluating oligonucleotide probe sequences so as to predict the potential for hybridization to a target nucleic acid sequence. The method of the application provides for the use of such methods to select preferred segments of a target mRNA sequence that are predicted to be single-stranded and, further, for the opportunistic utilization of the same or substantially identical target mRNA sequence, preferably comprising about 10-20 consecutive nucleotides of the target mRNA, in the design of both the RNAi oligonucleotides and ribozymes of the application.

A further aspect of the application relates to the use of the isolated “antisense” nucleic acids to inhibit expression, e.g., by inhibiting transcription and/or translation of a POSH or POSH-AP nucleic acid. The antisense nucleic acids may bind to the potential drug target by conventional base pair complementarity, or, for example, in the case of binding to DNA duplexes, through specific interactions in the major groove of the double helix. In general, these methods refer to the range of techniques generally employed in the art, and include any methods that rely on specific binding to oligonucleotide sequences.

An antisense construct of the present application can be delivered, for example, as an expression plasmid which, when transcribed in the cell, produces RNA which is complementary to at least a unique portion of the cellular mRNA which encodes a POSH or POSH-AP polypeptide. Alternatively, the antisense construct is an oligonucleotide probe, which is generated ex vivo and which, when introduced into the cell causes inhibition of expression by hybridizing with the mRNA and/or genomic sequences of a POSH or POSH-AP nucleic acid. Such oligonucleotide probes are preferably modified oligonucleotides, which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

With respect to antisense DNA, oligodeoxyribonucleotides derived from the translation initiation site, e.g., between the −10 and +10 regions of the target gene, are preferred. Antisense approaches involve the design of oligonucleotides (either DNA or RNA) that are complementary to mRNA encoding a POSH or POSH-AP polypeptide. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of a gene could be used in an antisense approach to inhibit translation of that mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the application. Whether designed to hybridize to the 5′, 3′ or coding region of mRNA, antisense nucleic acids should be at least six nucleotides in length, and are preferably less that about 100 and more preferably less than about 50, 25, 17 or 10 nucleotides in length.

It is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Results obtained using the antisense oligonucleotide may be compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published December 15, 1988) or the blood-brain barrier (see, e.g., PCT Publication No. WO89/10134, published Ap. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an alpha-anomeric oligonucleotide. An alpha-anomeric oligonucleotide forms specific double-stranded hybrids with complementary RNA in which, contrary to the usual antiparallel orientation, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

While antisense nucleotides complementary to the coding region of a POSH or POSH-AP mRNA sequence can be used, those complementary to the transcribed untranslated region may also be used.

In certain instances, it may be difficult to achieve intracellular concentrations of the antisense sufficient to suppress translation on endogenous mRNAs. Therefore a preferred approach utilizes a recombinant DNA construct in which the antisense oligonucleotide is placed under the control of a strong pol III or pol II promoter. The use of such a construct to transfect target cells will result in the transcription of sufficient amounts of single stranded RNAs that will form complementary base pairs with the endogenous potential drug target transcripts and thereby prevent translation. For example, a vector can be introduced such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct, which can be introduced directly into the tissue site.

Alternatively, POSH or POSH-AP gene expression can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C. 1991, Anticancer Drug Des., 6(6):569-84; Helene, C., et al., 1992, Ann. N.Y. Acad. Sci., 660:27-36; and Maher, L. J., 1992, Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, POSH or POSH-AP sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

A further aspect of the application relates to the use of DNA enzymes to inhibit expression of a POSH or POSH-AP gene. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid.

There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo include methods of delivery RNA ribozyme, as outlined in detail above. Additionally, one of skill in the art will recognize that, like antisense oligonucleotide, DNA enzymes can be optionally modified to improve stability and improve resistance to degradation.

Antisense RNA and DNA, ribozyme, RNAi and triple helix molecules of the application may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

6. Drug Screening Assays

In certain aspects, the present application provides assays for identifying therapeutic agents which either interfere with or promote POSH or POSH-AP function. In certain aspects, the present application also provides assays for identifying therapeutic agents which either interfere with or promote the complex formation between a POSH polypeptide and a POSH-AP polypeptide. In preferred embodiments of the application, the application provides assays for identifying therapeutic agents which either interfere with or promote POSH or POSH-AP (e.g., HERPUD1) function. In certain preferred aspects, the present application also provides assays for identifying therapeutic agents which either interfere with or promote the complex formation between a POSH polypeptide and a HERPUD1 polypeptide.

In preferred embodiments, the application provides agents for the treatment of neurological disorders. In certain embodiments, the application provides assays to identify, optimize or otherwise assess agents that disrupt the interaction between a POSH polypeptide and a HERPUD1 polypeptide. In certain preferred embodiments, an agent of the application is one that disrupts a complex comprising POSH and HERPUD1. Optionally, the agent is one that disrupts a complex comprising POSH and HERPUD1 without inhibiting POSH ubiquitin ligase activity, such as POSH auto-ubiquitination. In certain embodiments, an agent of the application is one that inhibits POSH-mediated ubiquitination of HERPUD1, optionally without inhibiting POSH auto-ubiquitination.

In certain embodiments, agents of the application are useful in treating or preventing neurological disorders. Treatment or prevention of a neurological disorder includes inhibition of the progression of a neurological disorder. In certain embodiments, an agent useful in the treatment or prevention of a neurological disorder interferes with the ubiquitin ligase catalytic activity of POSH (e.g., POSH ubiquitination of a target protein such as HERPUD1). In certain embodiments, an agent that inhibits the progression of a neurological disorder interferes with the ubiquitin ligase activity of POSH (e.g., POSH ubiquitination of a target protein such as HERPUD1). In other embodiments, agents disclosed herein inhibit or promote POSH and POSH-AP, such as HERPUD1, mediated cellular processes such as protein processing in the secretory pathway, for example, processing of amyloid polypeptides.

In certain embodiments, agents of the application are antiviral agents, optionally interfering with viral maturation, and preferably where the virus is an envelope virus, and optionally a retroid virus or an RNA virus. In certain embodiments, an antiviral agent interferes with the interaction between POSH and a POSH-AP polypeptide, for example an antiviral agent may disrupt an interaction between a POSH polypeptide and a HERPUD1 polypeptide. In yet additional embodiments, agents of the application interfere with the signaling of a GTPase, such as Rac or Ras, optionally disrupting the interaction between a POSH polypeptide and a Rac protein. In certain embodiments, agents of the application modulate the ubiquitin ligase activity of POSH and may be used to treat certain diseases related to ubiquitin ligase activity, such as various neurological disorders. In certain embodiments, agents of the application interfere with the trafficking of a protein through the secretory pathway.

In certain embodiments, the application provides assays to identify, optimize or otherwise assess agents that increase or decrease a ubiquitin-related activity of a POSH polypeptide. Ubiquitin-related activities of POSH polypeptides may include the self-ubiquitination activity of a POSH polypeptide, generally involving the transfer of ubiquitin from an E2 enzyme to the POSH polypeptide, and the ubiquitination of a target protein (e.g., HERPUD1), generally involving the transfer of a ubiquitin from a POSH polypeptide to the target protein. In certain embodiments, a POSH activity is mediated, at least in part, by a POSH RING domain.

In certain embodiments, an assay comprises forming a mixture comprising a POSH polypeptide, an E2 polypeptide and a source of ubiquitin (which may be the E2 polypeptide pre-complexed with ubiquitin). Optionally the mixture comprises an El polypeptide and optionally the mixture comprises a target polypeptide, such as, for example, HERPUD1. Additional components of the mixture may be selected to provide conditions consistent with the ubiquitination of the POSH polypeptide. One or more of a variety of parameters may be detected, such as POSH-ubiquitin conjugates, E2-ubiquitin thioesters, free ubiquitin and target polypeptide-ubiquitin complexes. The term “detect” is used herein to include a determination of the presence or absence of the subject of detection (e.g., POSH-ubiquitin, E2-ubiquitin, etc.), a quantitative measure of the amount of the subject of detection, or a mathematical calculation of the presence, absence or amount of the subject of detection, based on the detection of other parameters. The term “detect” includes the situation wherein the subject of detection is determined to be absent or below the level of sensitivity. Detection may comprise detection of a label (e.g., fluorescent label, radioisotope label, and other described below), resolution and identification by size (e.g., SDS-PAGE, mass spectroscopy), purification and detection, and other methods that, in view of this specification, will be available to one of skill in the art. For instance, radioisotope labeling may be measured by scintillation counting, or by densitometry after exposure to a photographic emulsion, or by using a device such as a Phosphorimager. Likewise, densitometry may be used to measure bound ubiquitin following a reaction with an enzyme label substrate that produces an opaque product when an enzyme label is used. In a preferred embodiment, an assay comprises detecting the POSH-ubiquitin conjugate.

In certain embodiments, an assay comprises forming a mixture comprising a POSH polypeptide, a target polypeptide and a source of ubiquitin (which may be the POSH polypeptide pre-complexed with ubiquitin). Optionally the mixture comprises an E1 and/or E2 polypeptide and optionally the mixture comprises an E2-ubiquitin thioester. Additional components of the mixture may be selected to provide conditions consistent with the ubiquitination of the target polypeptide. One or more of a variety of parameters may be detected, such as POSH-ubiquitin conjugates and target polypeptide-ubiquitin conjugates. In a preferred embodiment, an assay comprises detecting the target polypeptide-ubiquitin conjugate, such as, for example, detecting ubiquitinated HERPUD1. In another preferred embodiment, an assay comprises detecting the POSH-ubiquitin conjugate.

An assay described above may be used in a screening assay to identify agents that modulate a ubiquitin-related activity of a POSH polypeptide. A screening assay will generally involve adding a test agent to one of the above assays, or any other assay designed to assess a ubiquitin-related activity of a POSH polypeptide. The parameter(s) detected in a screening assay may be compared to a suitable reference. A suitable reference may be an assay run previously, in parallel or later that omits the test agent. A suitable reference may also be an average of previous measurements in the absence of the test agent In general the components of a screening assay mixture may be added in any order consistent with the overall activity to be assessed, but certain variations may be preferred. For example, in certain embodiments, it may be desirable to pre-incubate the test agent and the E3 (e.g., the POSH polypeptide), followed by removing the test agent and addition of other components to complete the assay. In this manner, the effects of the agent solely on the POSH polypeptide may be assessed.

In certain embodiments, an assay is performed in a high-throughput format. For example, one of the components of a mixture may be affixed to a solid substrate and one or more of the other components is labeled. For example, the POSH polypeptide may be affixed to a surface, such as a 96-well plate, and the ubiquitin is in solution and labeled. An E2 and E1 are also in solution, and the POSH-ubiquitin conjugate formation may be measured by washing the solid surface to remove uncomplexed labeled ubiquitin and detecting the ubiquitin that remains bound. Other variations may be used. For example, the amount of ubiquitin in solution may be detected. In certain embodiments, the formation of ubiquitin complexes may be measured by an interactive technique, such as FRET, wherein a ubiquitin is labeled with a first label and the desired complex partner (e.g., POSH polypeptide or target polypeptide) is labeled with a second label, wherein the first and second label interact when they come into close proximity to produce an altered signal. In FRET, the first and second labels are fluorophores. FRET is described in greater detail below. The formation of polyubiquitin complexes may be performed by mixing two or more pools of differentially labeled ubiquitin that interact upon formation of a polyubiqutin (see, e.g., US Patent Publication 20020042083). High-throughput may be achieved by performing an interactive assay, such as FRET, in solution as well. In addition, if a polypeptide in the mixture, such as the POSH polypeptide or target polypeptide, is readily purifiable (e.g., with a specific antibody or via a tag such as biotin, FIAG, polyhistidine, etc.), the reaction may be performed in solution and the tagged polypeptide rapidly isolated, along with any polypeptides, such as ubiquitin, that are associated with the tagged polypeptide. Proteins may also be resolved by SDS-PAGE for detection.

In certain embodiments, the ubiquitin is labeled, either directly or indirectly. This typically allows for easy and rapid detection and measurement of ligated ubiquitin, making the assay useful for high-throughput screening applications. As descrived above, certain embodiments may employ one or more tagged or labeled proteins. A “tag” is meant to include moieties that facilitate rapid isolation of the tagged polypeptide. A tag may be used to facilitate attachment of a polypeptide to a surface. A “label” is meant to include moieties that facilitate rapid detection of the labeled polypeptide. Certain moieties may be used both as a label and a tag (e.g., epitope tags that are readily purified and detected with a well-characterized antibody). Biotinylation of polypeptides is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known.

An “E1” is a ubiquitin activating enzyme. In a preferred embodiment, E1 is capable of transferring ubiquitin to an E2. In a preferred embodiment, E1 forms a high energy thiolester bond with ubiquitin, thereby “activating” the ubiquitin. An “E2” is a ubiquitin carrier enzyme (also known as a ubiquitin conjugating enzyme). In a preferred embodiment, ubiquitin is transferred from E1 to E2. In a preferred embodiment, the transfer results in a thiolester bond formed between E2 and ubiquitin. In a preferred embodiment, E2 is capable of transferring ubiquitin to a POSH polypeptide.

In an alternative embodiment, a POSH polypeptide, E2 or target polypeptide is bound to a bead, optionally with the assistance of a tag. Following ligation, the beads may be separated from the unbound ubiquitin and the bound ubiquitin measured. In a preferred embodiment, POSH polypeptide is bound to beads and the composition used includes labeled ubiquitin. In this embodiment, the beads with bound ubiquitin may be separated using a fluorescence-activated cell sorting (FACS) machine. Methods for such use are described in U.S. patent application Ser. No. 09/047,119, which is hereby incorporated in its entirety. The amount of bound ubiquitin can then be measured.

In a screening assay, the effect of a test agent may be assessed by, for example, assessing the effect of the test agent on kinetics, steady-state and/or endpoint of the reaction.

The components of the various assay mixtures provided herein may be combined in varying amounts. In a preferred embodiment, ubiquitin (or E2 complexed ubiquitin) is combined at a final concentration of from 5 to 200 ng per 100 microliter reaction solution. Optionally E1 is used at a final concentration of from 1 to 50 ng per 100 microliter reaction solution. Optionally E2 is combined at a final concentration of 10 to 100 ng per 100 microliter reaction solution, more preferably 10-50 ng per 100 microliter reaction solution. In a preferred embodiment, POSH polypeptide is combined at a final concentration of from 1 to 500 ng per 100 microliter reaction solution.

Generally, an assay mixture is prepared so as to favor ubiquitin ligase activity and/or ubiquitination activity. Generally, this will be physiological conditions, such as 50-200 mM salt (e.g., NaCl, KCl), pH of between 5 and 9, and preferably between 6 and 8. Such conditions may be optimized through trial and error. Incubations may be performed at any temperature which facilitates optimal activity, typically between 4 and 40° C. Incubation periods are selected for optimum activity, but may also be optimized to facilitate rapid high through put screening. Typically between 0.5 and 1.5 hours will be sufficient. A variety of other reagents may be included in the compositions. These include reagents like salts, solvents, buffers, neutral proteins, e.g., albumin, detergents, etc. which may be used to facilitate optimal ubiquitination enzyme activity and/or reduce non-specific or background interactions. Also reagents that otherwise improve the efficiency of the assay, such as protease inhibitors, nuclease inhibitors, anti-microbial agents, etc., may be used. The compositions will also preferably include adenosine tri-phosphate (ATP). The mixture of components may be added in any order that promotes ubiquitin ligase activity or optimizes identification of candidate modulator effects. In a preferred embodiment, ubiquitin is provided in a reaction buffer solution, followed by addition of the ubiquitination enzymes. In an alternate preferred embodiment, ubiquitin is provided in a reaction buffer solution, a candidate modulator is then added, followed by addition of the ubiquitination enzymes.

Test agents may be modified for use in vivo, e.g., by addition of a hydrophobic moiety, such as an ester.

An additional POSH-AP may be added to a POSH ubiquitination assay to assess the effect of the POSH-AP (e.g., HERPUD1) on POSH-mediated ubiquitination and/or to assess whether the POSH-AP (e.g., HERPUD1) is a target for POSH-mediated ubiquitination.

Certain embodiments of the application relate to assays for identifying agents that bind to a POSH or POSH-AP, such as HERPUD1, polypeptide, optionally a particular domain of POSH such as an SH3 or RING domain of a POSH polypeptide. A wide variety of assays may be used for this purpose, including labeled in vitro protein-protein binding assays, electrophoretic mobility shift assays, immunoassays for protein binding, and the like. The purified protein may also be used for determination of three-dimensional crystal structure, which can be used for modeling intermolecular interactions and design of test agents. In one embodiment, an assay detects agents which inhibit interaction of one or more subject POSH polypeptides with a POSH-AP, such as HERPUD1. In another embodiment, the assay detects agents which modulate the intrinsic biological activity of a POSH polypeptide or POSH complex, such as an enzymatic activity, binding to other cellular components, cellular compartmentalization, and the like.

In one aspect, the application provides methods and compositions for the identification of compositions that interfere with the function of POSH or POSH-AP polypeptides, such as HERPUD1 polypeptides.

A variety of assay formats will suffice and, in light of the present disclosure, those not expressly described herein will nevertheless be comprehended by one of ordinary skill in the art. Assay formats which approximate such conditions as formation of protein complexes, enzymatic activity, and even a POSH polypeptide-mediated membrane reorganization or vesicle formation activity, may be generated in many different forms, and include assays based on cell-free systems, e.g., purified proteins or cell lysates, as well as cell-based assays which utilize intact cells. Simple binding assays can also be used to detect agents which bind to POSH. Such binding assays may also identify agents that act by disrupting the interaction between a POSH polypeptide and a POSH interacting protein, such as a HERPUD1 protein, or the binding of a POSH polypeptide or complex to a substrate. Agents to be tested can be produced, for example, by bacteria, yeast or other organisms (e.g., natural products), produced chemically (e.g., small molecules, including peptidomimetics), or produced recombinantly. In a preferred embodiment, the test agent is a small organic molecule, e.g., other than a peptide or oligonucleotide, having a molecular weight of less than about 2,000 daltons.

In many drug screening programs which test libraries of compounds and natural extracts, high throughput assays are desirable in order to maximize the number of compounds surveyed in a given period of time. Assays of the present application which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

In preferred in vitro embodiments of the present assay, a reconstituted POSH complex comprises a reconstituted mixture of at least semi-purified proteins. By semi-purified, it is meant that the proteins utilized in the reconstituted mixture have been previously separated from other cellular or viral proteins. For instance, in contrast to cell lysates, the proteins involved in POSH complex formation are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure POSH complex assembly and/or disassembly.

Assaying POSH complexes, such as POSH:HERPUD1 complexes, in the presence and absence of a candidate inhibitor, can be accomplished in any vessel suitable for containing the reactants. Examples include microtitre plates, test tubes, and micro-centrifuge tubes.

In one embodiment of the present application, drug screening assays can be generated which detect inhibitory agents on the basis of their ability to interfere with assembly or stability of the POSH complex, such as the assembly or stability of a complex comprising one or more of a POSH polypeptide and a HERPUD1 polypeptide. In an exemplary binding assay, the compound of interest is contacted with a mixture comprising a POSH polypeptide and at least one interacting polypeptide, such as HERPUD1. Detection and quantification of POSH complexes provides a means for determining the compound's efficacy at inhibiting (or potentiating) interaction between the two polypeptides. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound.

Complex formation between the POSH polypeptides and a substrate polypeptide, such as HERPUD1, may be detected by a variety of techniques, many of which are effectively described above. For instance, modulation in the formation of complexes can be quantitated using, for example, detectably labeled proteins (e.g., radiolabeled, fluorescently labeled, or enzymatically labeled), by immunoassay, or by chromatographic detection. Surface plasmon resonance systems, such as those available from Biacore International AB (Uppsala, Sweden), may also be used to detect protein-protein interaction

Often, it will be desirable to immobilize one of the polypeptides to facilitate separation of complexes from uncomplexed forms of one of the proteins, as well as to accommodate automation of the assay. In an illustrative embodiment, a fusion protein can be provided which adds a domain that permits the protein to be bound to an insoluble matrix. For example, GST-POSH fusion proteins can be adsorbed onto glutathione sepharose beads (Sigma Chemical, St. Louis, Mo.) or glutathione derivatized microtitre plates, which are then combined with a potential interacting protein, e.g., an ³⁵S-labeled polypeptide, and the test compound and incubated under conditions conducive to complex formation. Following incubation, the beads are washed to remove any unbound interacting protein, and the matrix bead-bound radiolabel determined directly (e.g., beads placed in scintillant), or in the supernatant after the complexes are dissociated, e.g., when microtitre plate is used. Alternatively, after washing away unbound protein, the complexes can be dissociated from the matrix, separated by SDS-PAGE gel, and the level of interacting polypeptide found in the matrix-bound fraction quantitated from the gel using standard electrophoretic techniques.

In a further embodiment, agents that bind to a POSH or POSH-AP (e.g., HERPUD1) may be identified by using an immobilized POSH or POSH-AP. In an illustrative embodiment, a fusion protein can be provided which adds a domain that permits the protein to be bound to an insoluble matrix. For example, GST-POSH fusion proteins can be adsorbed onto glutathione sepharose beads (Sigma Chemical, St. Louis, Mo.) or glutathione derivatized microtitre plates, which are then combined with a potential labeled binding agent and incubated under conditions conducive to binding. Following incubation, the beads are washed to remove any unbound agent, and the matrix bead-bound label determined directly, or in the supernatant after the bound agent is dissociated.

In yet another embodiment, the POSH polypeptide and potential interacting polypeptide can be used to generate an interaction trap assay (see also, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696), for subsequently detecting-agents which disrupt binding of the proteins to one and other.

In particular, the method makes use of chimeric genes which express hybrid proteins. To illustrate, a first hybrid gene comprises the coding sequence for a DNA-binding domain of a transcriptional activator can be fused in frame to the coding sequence for a “bait” protein, e.g., a POSH polypeptide of sufficient length to bind to a potential interacting protein. The second hybrid protein encodes a transcriptional activation domain fused in frame to a gene encoding a “fish” protein, e.g., a potential interacting protein of sufficient length to interact with the POSH polypeptide portion of the bait fusion protein. If the bait and fish proteins are able to interact, e.g., form a POSH complex, they bring into close proximity the two domains of the transcriptional activator. This proximity causes transcription of a reporter gene which is operably linked to a transcriptional regulatory site responsive to the transcriptional activator, and expression of the reporter gene can be detected and used to score for the interaction of the bait and fish proteins.

One aspect of the present application provides reconstituted protein preparations including a POSH polypeptide and one or more interacting polypeptides.

In still further embodiments of the present assay, the POSH complex is generated in whole cells, taking advantage of cell culture techniques to support the subject assay. For example, as described below, the POSH complex can be constituted in a eukaryotic cell culture system, including mammalian and yeast cells. It may be desirable to express one or more viral proteins (e.g., Gag or Env) in such a cell along with a subject POSH polypeptide. It may also be desirable to infect the cell with a virus of interest Advantages to generating the subject assay in an intact cell include the ability to detect inhibitors which are functional in an environment more closely approximating that which therapeutic use of the inhibitor would require, including the ability of the agent to gain entry into the cell. Furthermore, certain of the in vivo embodiments of the assay, such as examples given below, are amenable to high through-put analysis of candidate agents.

The components of the POSH complex can be endogenous to the cell selected to support the assay. Alternatively, some or all of the components can be derived from exogenous sources. For instance, fusion proteins can be introduced into the cell by recombinant techniques (such as through the use of an expression vector), as well as by microinjecting the fusion protein itself or mRNA encoding the fusion protein.

In many embodiments, a cell is manipulated after incubation with a candidate agent and assayed for a POSH or POSH-AP activity. In certain embodiments a POSH or POSH-AP activity, such as HERPUD1 activity, is represented by production of virus like particles. As demonstrated herein, an agent that disrupts POSH or POSH-AP (e.g., HERPUD1) activity can cause a decrease in the production of virus like particles.

POSH complex formation may be assessed by immunoprecipitation and analysis of co-immunoprecipiated proteins or affinity purification and analysis of co-purified proteins. Fluorescence Resonance Energy Transfer (FRET)-based assays or other energy transfer assays may also be used to determine complex formation.

Additional bioassays for assessing POSH and POSH-AP activities may include assays to detect the improper processing of a protein that is associated with a neurological disorder. One assay that may be used is an assay to detect the presence, including an increase or a decrease in the amount, of a protein associated with a neurological disorder. For example, the use of RNAi may be employed to knockdown the expression of a POSH or POSH-AP polypeptide, such as HERPUD1, in cells (e.g., CHO cells, COS cells, or HeLa cells). The production of a secreted protein such as for example, amyloid beta, in the cell culture media, can then be assessed and compared to production of the secreted protein from control cells, which may be cells in which the POSH or POSH-AP activity (e.g., HERPUD1 activity) has not been inhibited. In some instances, a label may be incorporated into a secreted protein and the presence of the labeled secreted protein detected in the cell culture media. Proteins secreted from any cell type may be assessed, including for example, neural cells.

Bioassays for POSH or POSH-AP activities may include assays to detect the improper processing of a protein that is associated with a degenerative neurological disorder, such as Alzheimer's disease. One assay that may be used to detect POSH or POSH-AP activity associated with a neurological disorder is an assay to detect the presence, including an increase or a decrease in the amount, of amyloid polypeptides. One such assay includes assessing the effect of modulation of a POSH or POSH-AP on the production of amyloid polypeptides. For example, the use of RNAi may be employed to knockdown the expression of a POSH polypeptide or a POSH-AP (e.g., HERPUD1) in cells (e.g., HeLa cells) that express proteins associated with gamma-secretase activity, such as presenilin (e.g., presenilin 1), which enzymatic activity is involved in the proteolytic cleavage of amyloid beta precursor protein (“APP”) to yield amyloid beta peptide. Optionally, other proteins associated with gamma-secretase may be expressed, such as, for example, nicastrin, Aph-1, and Pen-2. The production of amyloid polypeptides, e.g., in the cell culture media, can then be assessed and compared to the production of amyloid polypeptides from cells in which the POSH or POSH-AP activity has not been modulated. In certain embodiments, the levels of APP can be assessed and compared to the levels of APP in which POSH or POSH-AP activity has not been modulated.

Additional assays for POSH or POSH-AP activities include in vitro gamma-secretase assays, which may be employed to assess the effect of modulation of a POSH or POSH-AP (e.g., knockdown of POSH expression or knockdown of HERPUD1 expression by RNAi) on gamma-secretase activity in comparison to the gamma-secretase activity in cells in which the POSH or POSH-AP activity has not been modulated. For example, gamma-secretase activity in the cells in which POSH or POSH-AP activity has been modulated (e.g., by RNAi) may be monitored by incubating solubilized gamma-secretase from the cells with tagged (e.g., a FLAG epitope) APP-based substrate and detecting the substrates and cleavage products (e.g., amyloid beta peptide) by immunoblotting and comparing the results to those of control cells (cells in which the POSH or POSH-AP activity has not been modulated) manipulated in the same manner. The effect of modulation of an activity of a POSH polypeptide or a POSH-AP on amyloid polypeptide production may be assessed in any cell capable of producing amyloid polypeptides.

The effect of an agent that modulates the activity of POSH or a POSH-AP, such as HERPUD1, may be evaluated for effects on mouse models of various neurological disorders. For example, mouse models of Alzheimer's disease have been described. See, for example, U.S. Pat. No. 5,612,486 for “Transgenic Animals Harboring APP Allele Having Swedish Mutation,” U.S. Pat. No. 5,850,003 (the '003 patent) for “Transgenic Rodents Harboring APP Allele Having Swedish Mutation,” and U.S. Pat. No. 5,455,169 entitled “Nucleic Acids for Diagnosing and Modeling Alzheimer's Disease”. Mouse models of Alzheimer's disease tend to produce elevated levels of beta-amyloid protein in the brain, and the increase or decrease of such protein in response to treatment with a test agent may be detected. In some instances, it may also be desirable to assess the effects of a test agent on cognitive or behavioral characteristics of a mouse model for Alzheimer's disease, as well as mouse models for other neurological disorders.

In a further embodiment, transcript levels may be measured in cells having higher or lower levels of POSH or POSH-AP activity, such as HERPUD1 activity, in order to identify genes that are regulated by POSH or POSH-APs. Promoter regions for such genes (or larger portions of such genes) may be operatively linked to a reporter gene and used in a reporter gene-based assay to detect agents that enhance or diminish POSH- or POSH-AP-regulated gene expression. Transcript levels may be determined in any way known in the art, such as, for example, Northern blotting, RT-PCR, microarray, etc. Increased POSH activity may be achieved, for example, by introducing a strong POSH expression vector. Decreased POSH activity may be achieved, for example, by RNAi, antisense, ribozyme, gene knockout, etc.

In general, where the screening assay is a binding assay (whether protein-protein binding, agent-protein binding, etc.), one or more of the molecules may be joined to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

In further embodiments, the application provides methods for identifying targets for therapeutic intervention. A polypeptide that interacts with POSH or participates in a POSH-mediated process (such as viral maturation) may be used to identify candidate therapeutics. Such targets may be identified by identifying proteins that associated with POSH (POSH-APs) by, for example, immunoprecipitation with an anti-POSH antibody, in silico analysis of high-throughput binding data, two-hybrid screens, and other protein-protein interaction assays described herein or otherwise known in the art in view of this disclosure. Agents that bind to such targets or disrupt protein-protein interactions thereof, or inhibit a biochemical activity thereof may be used in such an assay. Targets that have been identified by such approaches include HERPUD1.

A variety of other reagents may be included in the screening assay. These include reagents like salts, neutral proteins, e.g., albumin, detergents, etc that are used to facilitate optimal protein-protein binding and/or reduce nonspecific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4° C. and 40° C. Incubation periods are selected for optimum activity, but may also be optimized to facilitate rapid high-throughput screening.

In certain embodiments, a test agent may be assessed for antiviral activity by assessing effects on an activity (function) of a POSH-AP, such as, for example, POSH. Activity (function) may be affected by an agent that acts at one or more of the transcriptional, translational or post-translational stages. For example, an siRNA directed to a POSH-AP encoding gene will decrease activity, as will a small molecule that interferes with a catalytic activity of a POSH-AP. In certain embodiments, the agent inhibits the activity of one or more POSH polypeptides.

7. Exemplary Nucleic Acids and Expression Vectors

In certain aspects, the application relates to nucleic acids encoding POSH polypeptides and POSH-AP polypeptides, such as, for example, HERPUD1 polypeptides. For example, HERPUD1 polypeptides of the disclosure are listed in the Examples. Nucleic acid sequences encoding these HERPUD1 polypeptides are provided in the Examples. In certain embodiments, variants will also include nucleic acid sequences that will hybridize under highly stringent conditions to a nucleotide sequence of a coding sequence of a HERPUD1 polypeptide. Preferred nucleic acids of the application are human HERPUD1 sequences and variants thereof.

In certain aspects, the application relates to nucleic acids encoding POSH polypeptides. In preferred embodiments, the application relates to nucleic acids encoding POSH polypeptides, such as, for example, SEQ ID NOs: 2, 5, 7, 9, 11, 26, 27, 28, 29 and 30. Nucleic acids of the application are further understood to include nucleic acids that comprise variants of SEQ ID Nos:1, 3, 4, 6, 8, 10, 31, 32, 33, 34, and 35. Variant nucleotide sequences include sequences that differ by one or more nucleotide substitutions, additions or deletions, such as allelic variants; and will, therefore, include coding sequences that differ from the nucleotide sequence of the coding sequence designated in SEQ ID Nos:1, 3, 4, 6, 8 10, 31, 32, 33, 34, and 35, e.g., due to the degeneracy of the genetic code. In other embodiments, variants will also include sequences that will hybridize under highly stringent conditions to a nucleotide sequence of a coding sequence designated in any of SEQ ID Nos: 1, 3, 4, 6, 8 10, 31, 32, 33, 34, and 35. Preferred nucleic acids of the application are human POSH sequences, including, for example, any of SEQ ID Nos: 1, 3, 4, 6, 31, 32, 33, 34, 35 and variants thereof and nucleic acids encoding an amino acid sequence selected from among SEQ ID Nos: 2, 5, 7, 26, 27, 28, 29 and 30.

One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the application provides nucleic acids which hybridize under low stringency conditions of 6×SSC at room temperature followed by a wash at 2×SSC at room temperature.

Isolated nucleic acids which differ from the POSH nucleic acid sequences or from the POSH-AP nucleic acid sequences, such as the HERPUD1 nucleic acid sequences, due to degeneracy in the genetic code are also within the scope of the application. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this application.

Optionally, a POSH or a POSH-AP (e.g., HERPUD1) nucleic acid of the application will genetically complement a partial or complete loss of function phenotype in a cell. For example, a POSH nucleic acid of the application may be expressed in a cell in which endogenous POSH has been reduced by RNAi, and the introduced POSH nucleic acid will mitigate a phenotype resulting from the RNAi. An exemplary POSH loss of function phenotype is a decrease in virus-like particle production in a cell transfected with a viral vector, optionally an HIV vector.

Another aspect of the application relates to POSH and POSH-AP nucleic acids, such as HERPUD1 nucleic acids, that are used for antisense, RNAi or ribozymes. As used herein, nucleic acid therapy refers to administration or in situ generation of a nucleic acid or a derivative thereof which specifically hybridizes (e.g., binds) under cellular conditions with the cellular mRNA and/or genomic DNA encoding one of the POSH or POSH-AP, such as HERPUD1, polypeptides so as to inhibit production of that protein, e.g., by inhibiting transcription and/or translation. The binding may be by conventional base pair complementarity, or, for example, in the case of binding to DNA duplexes, through specific interactions in the major groove of the double helix.

A nucleic acid therapy construct of the present application can be delivered, for example, as an expression plasmid which, when transcribed in the cell, produces RNA which is complementary to at least a unique portion of the cellular mRNA which encodes a POSH or POSH-AP polypeptide, such as a HERPUD1 polypeptide. Alternatively, the construct is an oligonucleotide which is generated ex vivo and which, when introduced into the cell causes inhibition of expression by hybridizing with the mRNA and/or genomic sequences encoding a POSH or POSH-AP (e.g., HERPUD1) polypeptide. Such oligonucleotide probes are optionally modified oligonucleotide which are resistant to endogenous nucleases, e.g., exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., (1988) Biotechniques 6:958-976; and Stein et al., (1988) Cancer Res 48:2659-2668.

Accordingly, the modified oligomers of the application are useful in therapeutic, diagnostic, and research contexts. In therapeutic applications, the oligomers are utilized in a manner appropriate for nucleic acid therapy in general.

In another aspect of the application, the subject nucleic acid is provided in an expression vector comprising a nucleotide sequence encoding a POSH or POSH-AP, such as HERPUD1, polypeptide and operably linked to at least one regulatory sequence. Regulatory sequences are art-recognized and are selected to direct expression of the POSH or POSH-AP polypeptide. Accordingly, the term regulatory sequence includes promoters, enhancers and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a POSH or POSH-AP polypeptide. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

As will be apparent, the subject gene constructs can be used to cause expression of the POSH or POSH-AP polypeptides in cells propagated in culture, e.g., to produce proteins or polypeptides, including fusion proteins or polypeptides, for purification.

This application also pertains to a host cell transfected with a recombinant gene including a coding sequence for one or more of the POSH or POSH-AP (e.g., HERPUD1) polypeptides. The host cell may be any prokaryotic or eukaryotic cell. For example, a polypeptide of the present application may be expressed in bacterial cells such as E. coli, insect cells (e.g., using a baculovirus expression system), yeast, or mammalian cells. Other suitable host cells are known to those skilled in the art. Accordingly, the present application further pertains to methods of producing the POSH or POSH-AP (e.g., HERPUD1) polypeptides. For example, a host cell transfected with an expression vector encoding a POSH polypeptide can be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide. In a preferred embodiment, the POSH or POSH-AP polypeptide is a fusion protein containing a domain which facilitates its purification, such as a POSH-GST fusion protein, POSH-intein fusion protein, POSH-cellulose binding domain fusion protein, POSH-polyhistidine fusion protein etc.

A recombinant POSH or POSH-AP, such as HERPUD1, nucleic acid can be produced by ligating the cloned gene, or a portion thereof, into a vector suitable for expression in either prokaryotic cells, eukaryotic cells, or both. Expression vehicles for production of recombinant POSH or POSH-AP polypeptides include plasmids and other vectors. For instance, suitable vectors for the expression of a POSH polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant POSH or POSH-AP polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Alternatively, the coding sequences for the polypeptide can be incorporated as a part of a fusion gene including a nucleotide sequence encoding a different polypeptide. This type of expression system can be useful under conditions where it is desirable, e.g., to produce an immunogenic fragment of a POSH or POSH-AP (e.g., HERPUD1) polypeptide. For example, the VP6 capsid protein of rotavirus can be used as an immunologic carrier protein for portions of polypeptide, either in the monomeric form or in the form of a viral particle. The nucleic acid sequences corresponding to the portion of the POSH or POSH-AP polypeptide to which antibodies are to be raised can be incorporated into a fusion gene construct which includes coding sequences for a late vaccinia virus structural protein to produce a set of recombinant viruses expressing fusion proteins comprising a portion of the protein as part of the virion. The Hepatitis B surface antigen can also be utilized in this -role as well. Similarly, chimeric constructs coding for fusion proteins containing a portion of a POSH polypeptide and the poliovirus capsid protein can be created to enhance immunogenicity (see, for example, EP Publication NO: 0259149; and Evans et al., (1989) Nature 339:385; Huang et al., (1988) J. Virol. 62:3855; and Schlienger et al., (1992) J. Virol. 66:2).

The Multiple Antigen Peptide system for peptide-based immunization can be utilized, wherein a desired portion of a POSH or POSH-AP polypeptide is obtained directly from organo-chemical synthesis of the peptide onto an oligomeric branching lysine core (see, for example, Posnett et al., (1988) JBC 263:1719 and Nardelli et al., (1992) J. Immunol 148:914). Antigenic determinants of a POSH or POSH-AP polypeptide can also be expressed and presented by bacterial cells.

In another embodiment, a fusion gene coding for a purification leader sequence, such as a poly-(His)/enterolinase cleavage site sequence at the N-terminus of the desired portion of the recombinant protein, can allow purification of the expressed fusion protein by affinity chromatography using a Ni²⁺ metal resin. The purification leader sequence can then be subsequently removed by treatment with enterokinase to provide the purified POSH or POSH-AP polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

TABLE 2
Exemplary POSH nucleic acids

| | | Accession |
|---|---|---|
| Sequence Name | Organism | Number |
| CDNA FLJ11367 fis, clone | Homo sapiens | AK021429 |
| HEMBA1000303 | | |
| Plenty of SH3 domains | Mus musculus | NM_021506 |
| (POSH) mRNA | | |
| Plenty of SH3s (POSH) | Mus musculus | AF030131 |
| mRNA | | |
| Plenty of SH3s (POSH) | Drosophila melanogaster | NM_079052 |
| mRNA | | |
| Plenty of SH3s (POSH) | Drosophila melanogaster | AF220364 |
| mRNA | | |

TABLE 3
Exemplary POSH polypeptides

| Sequence Name | Organism | Accession Number |
|---|---|---|
| SH3 domains- | Mus musculus | T09071 |
| containing protein POSH | | |
| plenty of SH3 domains | Mus musculus | NP_067481 |
| Plenty of SH3s; POSH | Mus musculus | AAC40070 |
| Plenty of SH3s | Drosophila melanogaster | AAF37265 |
| LD45365p | Drosophila melanogaster | AAK93408 |
| POSH gene product | Drosophila melanogaster | AAF57833 |
| Plenty of SH3s | Drosophila melanogaster | NP_523776 |

In addition the following Tables provide the nucleic acid sequence and related SEQ ID NOs for domains of human POSH protein and a summary of POSH sequence identification numbers used in this application.

TABLE 4

Nucleic Acid Sequences and related
SEQ ID NOs for domains in human
POSH

| | |
|---|---|
| Name of | SEQ |
| the | ID |

| | | |
|---|---|---|
| sequence | Sequence | NO. |

| | | |
|---|---|---|
| RING | TGTCCGGTGTGTCTAGAGCGCCTTGATGCTTCTGCGAAG | 31 |
| domain | GTCTTTGCCTTGCCAGCATACGTTTTGCAAGCGATGTTT | |
| | GCTGGGGATCGTAGGTTCTCGAAATGAACTCAGATGTCC | |
| | CGAGT | |
| 1^{st} SH₃ | CCATGTGCCAAAGCGTTATACAACTATGAAGGAAAAGAG | 32 |
| domain | CCTGGAGACCTTAAATTCAGCAAAGGCGACATCATCATT | |
| | TTGCGAAGACAAGTGGATGAAAATTGGTACCATGGGGAA | |
| | GTCAATGGAATCCATGGCTTTTTCCCCACCAACTTTGTG | |
| | CAGATTATT | |
| 2^{nd} SH₃ | CCTCAGTGCAAAGCACTTTATGACTTTGAAGTGAAAGAC | 33 |
| domain | AAGGAAGCAGACAAAGATTGCCTTCCATTTGCAAAGGAT | |
| | GATGTTCTGACTGTGATCCGAAGAGTGGATGAAAACTGG | |
| | GCTGAAGGAATGCTGGCAGACAAAATAGGAATATTTCCA | |
| | ATTTCATATGTTGAGTTTAAC | |
| 3^{rd} SH₃ | AGTGTGTATGTTGCTATATATCCATACACTCCTCGGAAA | 34 |
| domain | GAGGATGAACTAGAGCTGAGAAAAGGGGAGATGTTTTTA | |
| | GTGTTTGAGCGCTGCCAGGATGGCTGGTTCAAAGGGACA | |
| | TCCATGCATACCAGCAAGATAGGGGTTTTCCCTGGCAAT | |
| | TATGTGGCACCAGTC | |
| 4^{th} SH₃ | GAAAGGCACAGGGTGGTGGTTTCCTATCCTCCTCAGAGT | 35 |
| domain | GAGGCAGAACTTGAACTTAAAGAAGGAGATATTGTGTTT | |
| | GTTCATAAAAAACGAGAGGATGGCTGGTTCAAAGGCACA | |
| | TTACAACGTAATGGGAAAACTGGCCTTTTCCCAGGAAGC | |
| | TTTGTGGAAAACA | |

TABLE 5
Summary of POSH sequence Identification Numbers

| | Sequence |
|---|---|
| | Identification |
| | Number |
| Sequence Information | (SEQ ID NO) |
| Human POSH Coding Sequence | SEQ ID No: 1 |
| Human POSH Amino Acid Sequence | SEQ ID No: 2 |
| Human POSH cDNA Sequence | SEQ ID No: 3 |
| 5′ cDNA Fragment of Human POSH | SEQ ID No: 4 |
| N-terminus Protein Fragment of Human POSH | SEQ ID No: 5 |
| 3′ mRNA Fragment of Human POSH | SEQ ID No: 6 |
| C-terminus Protein Fragment of Human POSH | SEQ ID No: 7 |
| Mouse POSH mRNA Sequence | SEQ ID No: 8 |
| Mouse POSH Protein Sequence | SEQ ID No: 9 |
| Drosophila melanogaster POSH mRNA Sequence | SEQ ID No: 10 |
| Drosophila melanogaster POSH Protein Sequence | SEQ ID No: 11 |
| Human POSH RING Domain Amino Acid Sequence | SEQ ID No: 26 |
| Human POSH 1^{st} SH₃ Domain Amino Acid Sequence | SEQ ID No: 27 |
| Human POSH 2^{nd} SH₃ Domain Amino Acid Sequence | SEQ ID No: 28 |
| Human POSH 3^{rd} SH₃ Domain Amino Acid Sequence | SEQ ID No: 29 |
| Human POSH 4^{th} SH₃ Domain Amino Acid Sequence | SEQ ID No: 30 |
| Human POSH RING Domain Nucleic Acid Sequence | SEQ ID No: 31 |
| Human POSH 1^{st} SH₃ Domain Nucleic Acid Sequence | SEQ ID No: 32 |
| Human POSH 2^{nd} SH₃ Domain Nucleic Acid Sequence | SEQ ID No: 33 |
| Human POSH 3^{rd} SH₃ Domain Nucleic Acid Sequence | SEQ ID No: 34 |
| Human POSH 4^{th} SH₃ Domain Nucleic Acid Sequence | SEQ ID No: 35 |

8. Exemplary Polypeptides

In certain aspects, the present application relates to POSH polypeptides, which are isolated from, or otherwise substantially free of, other intracellular proteins which might normally be associated with the protein or a particular complex including the protein. In certain embodiments, POSH polypeptides have an amino acid sequence that is at least 60% identical to an amino acid sequence as set forth in any of SEQ ID NOs: 2, 5, 7, 9, 11, 26, 27, 28, 29 and 30. In other embodiments, the polypeptide has an amino acid sequence at least 65%, 70%, 75%, 80%, 85%, 90%, 95%, 97%, 98%, 99% or 100% identical to an amino acid sequence as set forth in any of SEQ ID NOs: 2, 5, 7, 9, 11, 26, 27, 28, 29 and 30.

In certain aspects, the application relates to POSH-AP polypeptides. In preferred embodiments, the present application relates to the POSH-AP, HERPUD1, polypeptides, which are isolated from, or otherwise substantially free of, other intracellular proteins which might normally be associated with the protein or a particular complex including the protein.

Optionally, a POSH or POSH-AP polypeptide of the application will function in place of an endogenous POSH or POSH-AP polypeptide, for example by mitigating a partial or complete loss of function phenotype in a cell. For example, a POSH polypeptide of the application may be produced in a cell in which endogenous POSH has been reduced by RNAi, and the introduced POSH polypeptide will mitigate a phenotype resulting from the RNAi. An exemplary POSH loss of function phenotype is a decrease in virus-like particle production in a cell transfected with a viral vector, optionally an HIV vector.

In another aspect, the application provides polypeptides that are agonists or antagonists of a POSH or POSH-AP polypeptide. Variants and fragments of a POSH or POSH-AP polypeptide may have a hyperactive or constitutive activity, or, alternatively, act to prevent POSH or POSH-AP polypeptides from performing one or more functions. For example, a mutant form of a POSH or POSH-AP protein domain may have a dominant negative effect.

Another aspect of the application relates to polypeptides derived from a full-length POSH or POSH-AP (e.g., HERPUD1) polypeptide. Isolated peptidyl portions of the subject proteins can be obtained by screening polypeptides recombinantly produced from the corresponding fragment of the nucleic acid encoding such polypeptides. In addition, fragments can be chemically synthesized using techniques known in the art such as conventional Merrifield solid phase f-Moc or t-Boc chemistry. For example, any one of the subject proteins can be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of the formation of a specific protein complex, or more generally of a POSH:POSH-AP complex, such as by microinjection assays.

It is also possible to modify the structure of the POSH or POSH-AP polypeptides for such purposes as enhancing therapeutic or prophylactic efficacy, or stability (e.g., ex vivo shelf life and resistance to proteolytic degradation in vivo). Such modified polypeptides, when designed to retain at least one activity of the naturally-occurring form of the protein, are considered functional equivalents of the POSH or POSH-AP (e.g., HERPUD1) polypeptides described in more detail herein. Such modified polypeptides can be produced, for instance, by amino acid substitution, deletion, or addition.

For instance, it is reasonable to expect, for example, that an isolated replacement of a leucine with an isoleucine or valine, an aspartate with a glutamate, a threonine with a serine, or a similar replacement of an amino acid with a structurally related amino acid (i.e, conservative mutations) will not have a major effect on the biological activity of the resulting molecule. Conservative replacements are those that take place within a family of amino acids that are related in their side chains. Genetically encoded amino acids are can be divided into four families (see, for example, Biochemistry, 2nd ed., Ed. by L. Stryer, W.H. Freeman and Co., 1981). Whether a change in the amino acid sequence of a polypeptide results in a functional homolog can be readily determined by assessing the ability of the variant polypeptide to produce a response in cells in a fashion similar to the wild-type protein. For instance, such variant forms of a POSH polypeptide can be assessed, e.g., for their ability to bind to another polypeptide, e.g., another POSH polypeptide or another protein involved in the generation of beta-amyloid peptides, such as the POSH-AP, HERPUD1. Polypeptides in which more than one replacement has taken place can readily be tested in the same manner.

This application further contemplates a method of generating sets of combinatorial mutants of the POSH or POSH-AP polypeptides, as well as truncation mutants, and is especially useful for identifying potential variant sequences (e.g., homologs) that are functional in binding to a POSH or POSH-AP polypeptide. The purpose of screening such combinatorial libraries is to generate, for example, POSH homologs which can act as either agonists or antagonist, or alternatively, which possess novel activities all together. Combinatorially-derived homologs can be generated which have a selective potency relative to a naturally occurring POSH or POSH-AP polypeptide. Such proteins, when expressed from recombinant DNA constructs, can be used in gene therapy protocols.

Likewise, mutagenesis can give rise to homologs which have intracellular half-lives dramatically different than the corresponding wild-type protein. For example, the altered protein can be rendered either more stable or less stable to proteolytic degradation or other cellular process which result in destruction of, or otherwise inactivation of the POSH or POSH-AP polypeptide of interest. Such homologs, and the genes which encode them, can be utilized to alter POSH or POSH-AP levels by modulating the half-life of the protein. For instance, a short half-life can give rise to more transient biological effects and, when part of an inducible expression system, can allow tighter control of recombinant POSH or POSH-AP levels within the cell. As above, such proteins, and particularly their recombinant nucleic acid constructs, can be used in gene therapy protocols.

In similar fashion, POSH or POSH-AP homologs can be generated by the present combinatorial approach to act as antagonists, in that they are able to interfere with the ability of the corresponding wild-type protein to function.

In a representative embodiment of this method, the amino acid sequences for a population of POSH or POSH-AP homologs are aligned, preferably to promote the highest homology possible. Such a population of variants can include, for example, homologs from one or more species, or homologs from the same species but which differ due to mutation. Amino acids which appear at each position of the aligned sequences are selected to create a degenerate set of combinatorial sequences. In a preferred embodiment, the combinatorial library is produced by way of a degenerate library of genes encoding a library of polypeptides which each include at least a portion of potential POSH or POSH-AP sequences. For instance, a mixture of synthetic oligonucleotides can be enzymatically ligated into gene sequences such that the degenerate set of potential POSH or POSH-AP nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential POSH or POSH-AP sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. A G Walton, Amsterdam: Elsevier pp 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, POSH or POSH-AP homologs (both agonist and antagonist forms) can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of POSH or POSH-AP polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of POSH or POSH-AP homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In an illustrative embodiment of a screening assay, candidate combinatorial gene products of one of the subject proteins are displayed on the surface of a cell or virus, and the ability of particular cells or viral particles to bind a POSH or POSH-AP polypeptide is detected in a “panning assay”. For instance, a library of POSH variants can be cloned into the gene for a surface membrane protein of a bacterial cell (Ladner et al., WO 88/06630; Fuchs et al., (1991) Bio/Technology 9:1370-1371; and Goward et al., (1992) TIBS 18:136-140), and the resulting fusion protein detected by panning, e.g., using a fluorescently labeled molecule which binds the POSH polypeptide, to score for potentially functional homologs. Cells can be visually inspected and separated under a fluorescence microscope, or, where the morphology of the cell permits, separated by a fluorescence-activated cell sorter.

In similar fashion, the gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and fl are most often used in phage display libraries, as either of the phage gIII or gVIII coat proteins can be used to generate fusion proteins without disrupting the ultimate packaging of the viral particle (Ladner et al., PCT publication WO 90/02909; Garrard et al., PCT publication WO 92/09690; Marks et al., (1992) J. Biol. Chem. 267:16007-16010; Griffiths et al., (1993) EMBO J. 12:725-734; Clackson et al., (1991) Nature 352:624-628; and Barbas et al., (1992) PNAS USA 89:4457-4461).

The application also provides for reduction of the POSH or POSH-AP polypeptides to generate minetics, e.g., peptide or non-peptide agents, which are able to mimic binding of the authentic protein to another cellular partner. Such mutagenic techniques as described above, as well as the thioredoxin system, are also particularly useful for mapping the determinants of a POSH or POSH-AP polypeptide which participate in protein-protein interactions involved in, for example, binding of proteins involved in viral maturation to each other. To illustrate, the critical residues of a POSH or POSH-AP polypeptide which are involved in molecular recognition of a substrate protein can be determined and used to generate its derivative peptidomimetics which bind to the substrate protein, and by inhibiting POSH or POSH-AP binding, act to inhibit its biological activity. By employing, for example, scanning mutagenesis to map the amino acid residues of a POSH polypeptide which are involved in binding to another polypeptide, peptidomimetic compounds can be generated which mimic those residues involved in binding. For instance, non-hydrolyzable peptide analogs of such residues can be generated using benzodiazepine (e.g., see Freidinger et al., in Peptides: Chemistry and Biology, G. R. Marshall ed., ESCOM Publisher: Leiden, Netherlands, 1988), azepine (e.g., see Huffinan et al., in Peptides: Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., (1985) Tetrahedron Lett 26:647; and Sato et al., (1986) J Chem Soc Perkin Trans 1:1231), and b-aminoalcohols (Gordon et al., (1985) Biochem Biophys Res Commun 126:419; and Dann et al., (1986) Biochem Biophys Res Commun 134:71).

The following table provides the sequences of the RING domain and the various SH3 domains of POSH.

TABLE 6

Amino Acid Sequences and related SEQ ID NOs
for domains in human POSH

| | |
|---|---|
| Name of | SEQ |
| the | ID |

| | | |
|---|---|---|
| sequence | Sequence | NO. |

| | | |
|---|---|---|
| RING | CPVCLERLDASAKVLPCQHTFCKRCLLGIVGSRNELRCP | 26 |
| domain | EC | |
| 1^{st} SH₃ | PCAKALYNYEGKEPGDLKFSKGDIIILRRQVDENWYHGE | 27 |
| domain | EC | |
| 2^{nd} SH₃ | PQCKALYDFEVKDKEADKDCLPFAKDDVLTVIRRVDENW | 28 |
| domain | AEGMLADKIGIFPISYVEFNS | |
| 3^{rd} SH₃ | SVYVAIYPYTPRKEDELELRKGEMFLVFERCQDGWFKGT | 29 |
| domain | SMHTSKIGVFPGNYVAPVT | |
| 4^{th} SH₃ | ERHRVVVSYPPQSEAELELKEGDIVFVHKKREDGWFKGT | 30 |
| domain | LQRNGKTGLFPGSFVENI | |

10. Effective Dose

Toxicity and therapeutic efficacy of such compounds can be determined by standard pharmaceutical procedures in cell cultures or experimental animals, e.g., for determining The LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exlubit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the application, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

11. Formulation and Use

Pharmaceutical compositions for use in accordance with the present application may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

An exemplary composition of the application comprises an RNAi mixed with a delivery system, such as a liposome system, and optionally including an acceptable excipient. In a preferred embodiment, the composition is formulated for topical administration for, e.g., herpes virus infections.

For such therapy, the compounds of the application can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the application can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present application are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. in addition, detergents may be used to facilitate permeation. Transmucosal administration may be through nasal sprays or using suppositories. For topical administration, the oligomers of the application are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

For therapies involving the administration of nucleic acids, the oligomers of the application can be formulated for a variety of modes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, intranodal, and subcutaneous for injection, the oligomers of the application can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, the oligomers of the application are formulated into ointments, salves, gels, or creams as generally known in the art.

The application now being generally described, it will be more readily understood by reference to the following examples, which are included merely for purposes of illustration of certain aspects and embodiments of the present application, and are not intended to limit the application.

### EXAMPLES

### Example 1

### Role of POSH in Virus-Like Particle (VLP) Budding

1. Objective:

Use RNAi to inhibit POSH gene expression and compare the efficiency of viral budding and GAG expression and processing in treated and untreated cells.

2. Study Plan:

HeLa SS-6 cells are transfected with mRNA-specific RNAi in order to knockdown the target proteins. Since maximal reduction of target protein by RNAi is achieved after 48 hours, cells are transfected twice—first to reduce target mRNAs, and subsequently to express the viral Gag protein. The second transfection is performed with pNLenv (plasmid that encodes HIV) and with low amounts of RNAi to maintain the knockdown of target protein during the time of gag expression and budding of VLPs. Reduction in mRNA levels due to RNAi effect is verified by RT-PCR amplification of target mRNA.

3. Methods, Materials, Solutions

a. Methods
i. Transfections according to manufacturer's protocol and as described in procedure.
ii. Protein determined by Bradford assay.
iii. SDS-PAGE in Hoeffer miniVE electrophoresis system. Transfer in Bio-Rad mini-protean II wet transfer system. Blots visualized using Typhoon system, and ImageQuant software (ABbiotech)

b. Materials

| Material | Manufacturer | Catalog # | Batch # |
|---|---|---|---|
| Lipofectamine 2000 | Life | 11668-019 | 1112496 |
| (LF2000) | Technologies | | |
| OptiMEM | Life | 31985-047 | 3063119 |
| | Technologies | | |
| RNAi Lamin A/C | Self | 13 | |
| RNAi TSG101 688 | Self | 65 | |
| RNAi Posh 524 | Self | 81 | |
| plenvl1 PTAP | Self | 148 | |
| plenvl1 ATAP | Self | 149 | |
| Anti-p24 polyclonal | Seramun | | A-0236/ |
| antibody | | | 5-10-01 |
| Anti-Rabbit Cy5 | Jackson | 144-175-115 | 48715 |
| conjugated antibody | | | |
| 10% acrylamide Tris- | Life | NP0321 | 1081371 |
| Glycine SDS-PAGE gel | Technologies | | |
| Nitrocellulose | Schleicher & | 401353 | BA-83 |
| membrane | Schuell | | |
| NuPAGE 20× transfer | Life | NP0006-1 | 224365 |
| buffer | Technologies | | |
| 0.45 μm filter | Schleicher & | 10462100 | CS1018-1 |
| | Schuell | | |

c. Solutions

| | Compound | Concentration |
|---|---|---|
| Lysis Buffer | Tris-HCl pH 7.6 | 50 mM |
| | MgCl₂ | 15 mM |
| | NaCl | 150 mM |
| | Glycerol | 10% |
| | EDTA | 1 mM |
| | EGTA | 1 mM |
| | ASB-14 | 1% |
| | (add immediately before use) | |
| 6× Sample | Tris-HCl, pH = 6.8 | 1M |
| Buffer | Glycerol | 30% |
| | SDS | 10% |
| | DTT | 9.3% |
| | Bromophenol Blue | 0.012% |
| TBS-T | Tris pH = 7.6 | 20 mM |
| | NaCl | 137 mM |
| | Tween-20 | 0.1% |

4. Procedure

a. Schedule

Day

| 1 | 2 | 3 | 4 | 5 |
|---|---|---|---|---|
| Plate | Transfection | Passage | Transfection II | Extract RNA |
| cells | I | cells | (RNAi and | for RT-PCR |
| | (RNAi only) | (1:3) | pNlenv) | (post |
| | | | (12:00, PM) | transfection) |
| | | | Extract RNA for | Harvest VLPs |
| | | | RT-PCR | and cells |
| | | | (pre-transfection) | |

b. Day 1

Plate HeLa SS-6 cells in 6-well plates (35mm wells) at concentration of 5×10⁵ cells/well.

c. Day2

2 hours before transfection replace growth medium with 2 ml growth medium without antibiotics.

Transfection I:

| | | | | | RNAi | A | B |
|---|---|---|---|---|---|---|---|
| | | | | | [20 μM] | OPtiMEM | LF2000 mix |
| Reaction | RNAi name | TAGDA# | Reactions | RNAi [nM] | μl | (μl) | (μl) |
| 1 | Lamin A/C | 13 | 2 | 50 | 12.5 | 500 | 500 |
| 2 | Lamin A/C | 13 | 1 | 50 | 6.25 | 250 | 250 |
| 3 | TSG101 688 | 65 | 2 | 20 | 5 | 500 | 500 |
| 5 | Posh 524 | 81 | 2 | 50 | 12.5 | 500 | 500 |

Transfections:
Prepare LF2000 mix: 250 μl OptiMEM+5 μl LF2000 for each reaction. Mix by inversion, 5 times. Incubate 5 minutes at room temperature.
Prepare RNA dilution in OptiMEM (Table 1, column A). Add LF2000 mix dropwise to diluted RNA (Table 1, column B). Mix by gentle vortex. Incubate at room temperature 25 minutes, covered with aluminum foil.
Add 500 μl transfection mixture to cells dropwise and mix by rocking side to side.
Incubate overnight.

d. Day 3
Split 1:3 after 24 hours. (Plate 4 wells for each reaction, except reaction 2 which is plated into 3 wells.)

e. Day 4

2 hours pre-transfection replace medium with DMEM growth medium without antibiotics.

Transfection II

| | | | | | B | | |
|---|---|---|---|---|---|---|---|
| | | | | A | RNAi | | |
| | | | | Plasmid | [20 μM] | C | D |
| | TAG | | Reaction | for 2.4 μg | for 10 nM | OPtiMEM | LF2000 mix |
| RNAi name | DA# | Plasmid | # | (μl) | (μl) | (μl) | (μl) |
| Lamin A/C | 13 | PTAP | 3 | 3.4 | 3.75 | 750 | 750 |
| Lamin A/C | 13 | ATAP | 3 | 2.5 | 3.75 | 750 | 750 |
| TSG101 688 | 65 | PTAP | 3 | 3.4 | 3.75 | 750 | 750 |
| Posh 524 | 81 | PTAP | 3 | 3.4 | 3.75 | 750 | 750 |

Prepare LF2000 mix: 250 μl OptiMEM+5 μl LF2000 for each reaction. Mix by inversion, 5 times. Incubate 5 minutes at room temperature.
Prepare RNA+DNA diluted in OptiMEM (Transfection II, A+B+C)
Add LF2000 mix (Transfection II, D) to diluted RNA+DNA dropwise, mix by gentle vortex, and incubate 1 h while protected from light with aluminum foil.
Add LF2000 and DNA+RNA to cells, 500μl/well, mix by gentle rocking and incubate overnight.

f. Day 5
Collect samples for VLP assay (approximately 24 hours post-transfection) by the following procedure (cells from one well from each sample is taken for RNA assay, by RT-PCR).

g. Cell Extracts
i. Pellet floating cells by centrifugation (5 min, 3000 rpm at 4° C.), save supernatant (continue with supernatant immediately to step h), scrape remaining cells in the medium which remains in the well, add to the corresponding floating cell pellet and centrifuge for 5 minutes, 1800 rpm at 4° C.
ii. Wash cell pellet twice with ice-cold PBS.
iii. Resuspend cell pellet in 100 μl lysis buffer and incubate 20 minutes on ice.
iv. Centrifuge at 14,000 rpm for 15 min. Transfer supernatant to a clean tube. This is the cell extract.
v. Prepare 10 μl of cell extract samples for SDS-PAGE by adding SDS-PAGE sample buffer to 1×, and boiling for 10 minutes. Remove an aliquot of the remaining sample for protein determination to verify total initial starting material. Save remaining cell extract at −80° C.

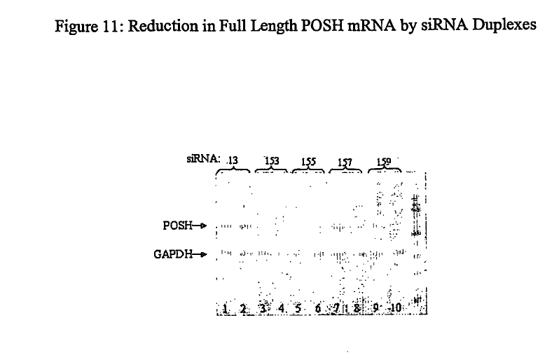

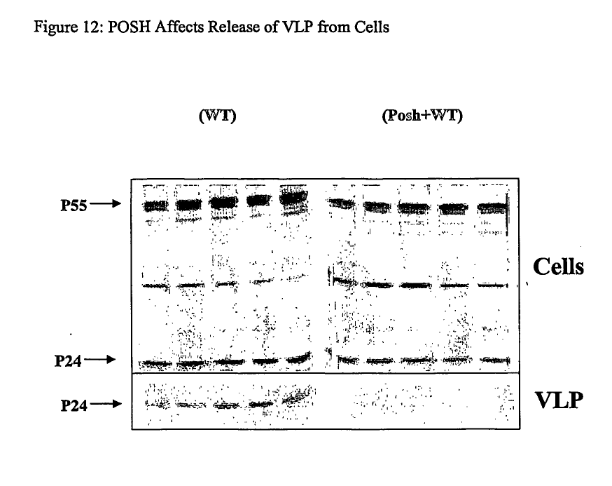

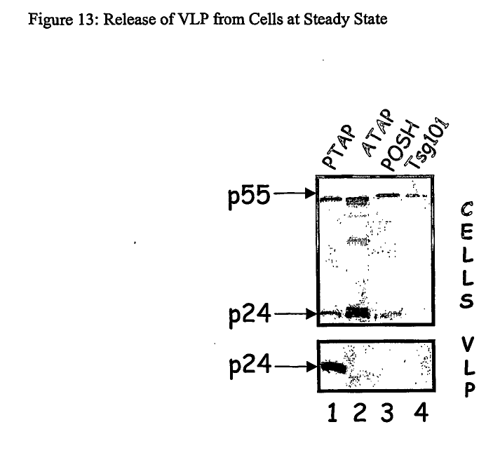

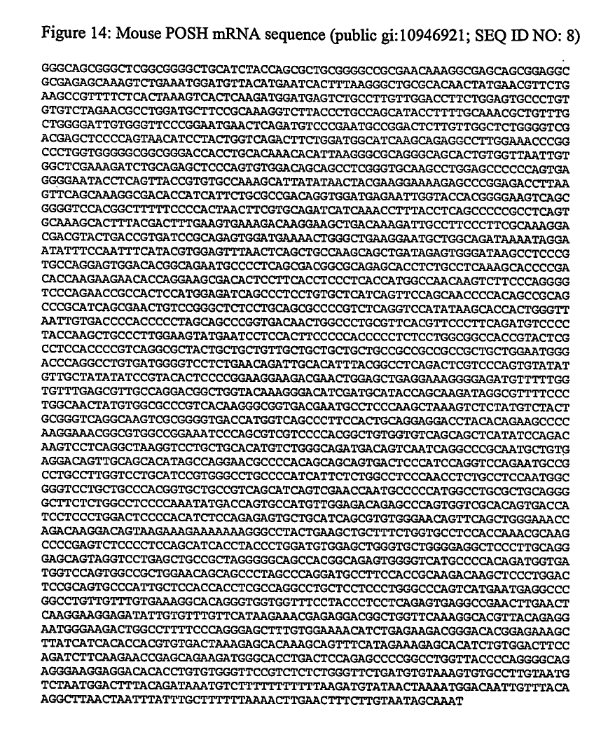

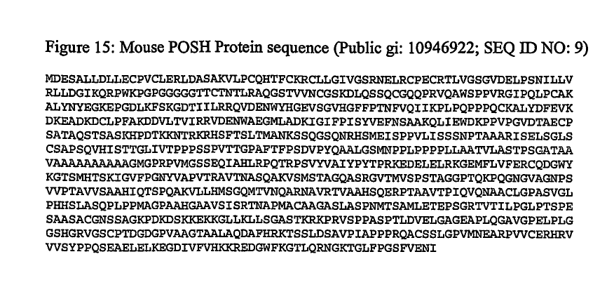

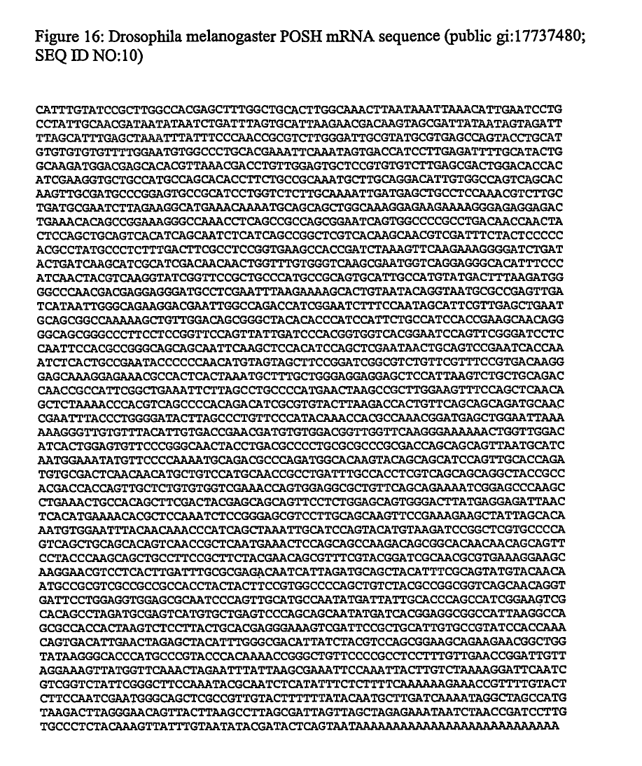

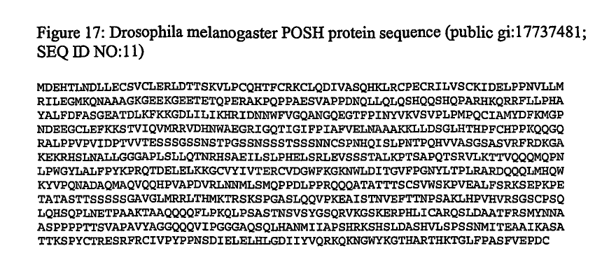

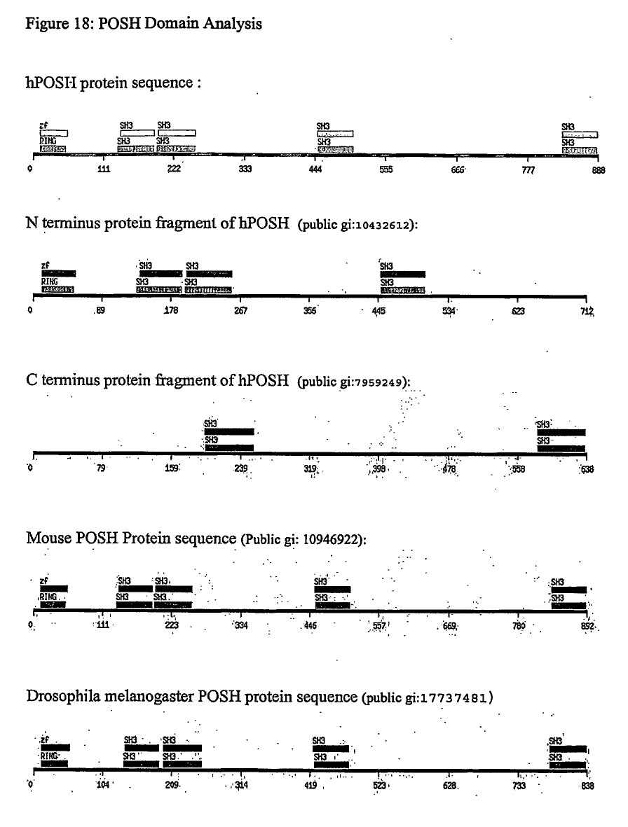

h. Purification of VLPs from Cell Cedia
i. Filter the supernatant from step g through a 0.45 m filter.
ii. Centrifuge supernatant at 14,000 rpm at 4° C. for at least 2 h.
iii. Aspirate supernatant carefully.
iv. Re-suspend VLP pellet in hot (100° C. warmed for 10 min at least) 1× sample buffer.
v. Boil samples for 10 minutes, 100° C.
   i. Western Blot Analysis
i. Run all samples from stages A and B on Tris-Glycine SDS-PAGE 10% (120V for 1.5 h).
ii. Transfer samples to nitrocellulose membrane (65V for 1.5 h).
iii. Stain membrane with ponceau S solution.
iv. Block with 10% low fat milk in TBS-T for 1 h.
v. Incubate with anti p24 rabbit 1:500 in TBS-T o/n.
vi. Wash 3 times with TBS-T for 7 min each wash.
vii. Incubate with secondary antibody anti rabbit cy5 1:500 for 30 min.
viii. Wash five times for 10 min in TBS-T.
ix. View in Typhoon gel imaging system (Molecular Dynamics/APBiotech) for fluorescence signal.
   Results are shown in FIGS. 11-13.

### Example 2

### Exemplary POSH RT-PCR Primers and siRNA Duplexes

RT-PGR primers

| | | |
|---|---|---|
| Name | Position | Sequence |

| | | | |
|---|---|---|---|
| Sense | POSH = 271 | 271 | 5′ CTTGCCTTGCCAGCATAC 3′ |
| primer | | | (SEQ ID NO:12) |
| Anti- | POSH = 926c | 926C | 5′ CTGCCAGCATTCCTTCAG 3′ |
| sense | | | (SEQ ID NO:13) |
| primer | | | |

siRNA Duplexes:

| | |
|---|---|
| siRNA No: | |
| 153 | |
| siRNA Name: | |
| POSH-230 | |
| Position in mRNA | |
| 426-446 | |
| Target sequence: | |
| 5′ AACAGAGGCCTTGGAAACCTG 3′ | SEQ ID NO: 14 |
| siRNA sense strand: | |
| 5′ dTdTCAGAGGCCUUGGAAACCUG 3′ | SEQ ID NO: 15 |
| siRNA anti-sense strand: | |
| 5′dTdTCAGGUUUCCAAGGCCUCUG 3′ | SEQ ID NO: 16 |
| siRNA No: | |
| 155 | |
| siRNA Name: | |
| POSH-442 | |
| Position in mRNA | |
| 638-658 | |
| Target sequence: | |
| 5′ AAAGAGCCTGGAGACCTTAAA 3′ | SEQ ID NO: 17 |
| siRNA sense strand: | |
| 5′ ddTdTAGAGCCUGGAGACCUUAAA 3′ | SEQ ID NO: 18 |
| siRNA anti-sense strand: | |
| 5′ ddTdTUUUAAGGUCUCCAGGCUCU 3′ | SEQ ID NO: 19 |
| siRNA No: | |
| 157 | |
| siRNA Name: | |
| POSH-U111 | |
| Position in mRNA | |
| 2973-2993 | |
| Target sequence: | |
| 5′ AAGGATTGGTATGTGACTCTG 3′ | SEQ ID NO: 20 |
| siRNA sense strand: | |
| 5′ dTdTGGAUUGGUAGUGACUCUG 3′ | SEQ ID NO: 21 |
| siRNA anti-sense strand: | |
| 5′ dTdTCAGAGUCACAUACCAAUCC 3′ | SEQ ID NO: 22 |
| siRNA No: | |
| 159 | |
| siRNA Name: | |
| POSH-U410 | |
| Position in mRNA | |
| 3272-3292 | |
| Target sequence: | |
| 5′ AAGCTGGATTATCTCCTGTTG 3′ | SEQ ID NO: 23 |
| siRNA sense strand: | |
| 5′ ddTdTGCUGGAUUAUCUCCUGUUG 3′ | SEQ ID NO: 24 |
| siRNA anti-sense strand: | |
| 5′ ddTdTCAACAGGAGAUAAUCCAGC 3′ | SEQ ID NO: 25 |
| siRNA No.: | |
| 187 | |
| siRNA Name: | |
| POSH-control | |
| Position in mRNA: | |
| None. Reverse to #153 | |
| Target sequence: | |
| 5′ AAGTCCAAAGGTTCCGGAGAC 3′ | SEQ ID NO: 36 |

### Example 3

### Knock-Down of hPOSH Entraps HIV Virus Particles in Intracellular Vesicles

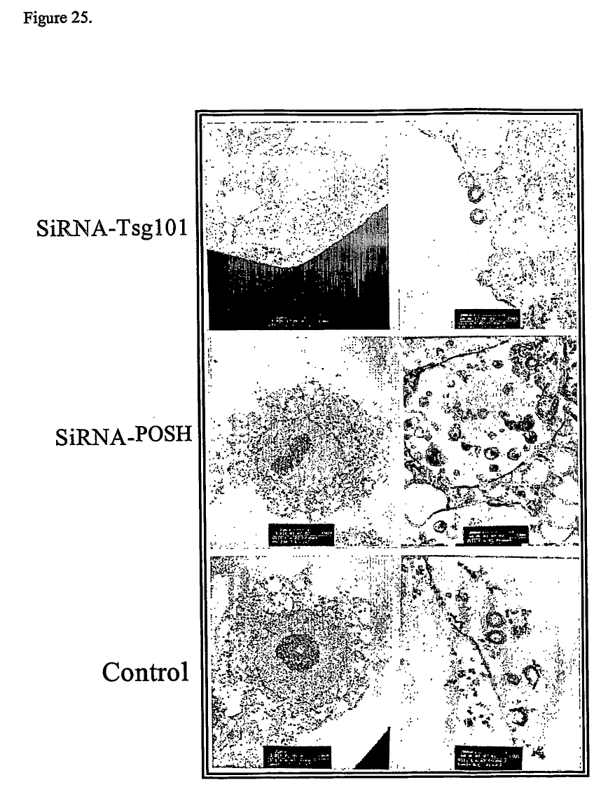

HIV virus release was analyzed by electron microscopy following siRNA and full-length HIV plasmid (missing the envelope coding region) transfection. Mature viruses were secreted by cells transfected with HIV plasmid and non-relevant siRNA (control, lower panel). Knockdown of Tsg101 protein resulted in a budding defect, the viruses that were released had an immature phenotype (upper panel). Knockdown of hPOSH levels resulted in accumulation of viruses inside the cell in intracellular vesicles (middle panel). Results, shown in FIG. 25, indicate that inhibiting hPOSH entraps HIV virus particles in intracellular vesicles. As accumulation of HIV virus particles in the cells accelerate cell death, inhibition of hPOSH therefore destroys HIV reservoir by killing cells infected with HIV.

### Example 4

### In-vitro Assay of Human POSH Self-Ubiquitination

Recombinant hPOSH was incubated with ATP in the presence of E1, E2 and ubiquitin as indicated in each lane. Following incubation at 37° C. for 30 minutes, reactions were terminated by addition of SDS-PAGE sample buffer. The samples were subsequently resolved on a 10% polyacrylamide gel. The separated samples were then transferred to nitrocellulose and subjected to immunoblot analysis with an anti ubiquitin polyclonal antibody. The position of migration of molecular weight markers is indicated on the right.

Poly-Ub: Ub-hPOSHconjugates, detected as high molecular weight adducts only in reactions containing E1, E2 and ubiquitin. hPOSH-176 and hPOSH-178 are a short and a longer derivatives (respectively) of bacterially expressed hPOSH; C, control E3.

Preliminary Steps in a High-Throughput Screen

Materials

1. E1 recombinant from bacculovirus
2. E2 Ubch5c from bacteria
3. Ubiquitin
4. POSH #178 (1-361) GST fusion-purified but degraded
5. POSH # 176 (1-269) GST fusion-purified but degraded
6. hsHRD1 soluble ring containing region
5. Bufferx12 (Tris 7.6 40 mM, DTT 1 mM, MgCl₂ 5mM, ATP 2 uM)

6. Dilution buffer (Tris 7.6 40 mM, DTT 1 mM, ovalbumin 1 ug/ul) protocol

| | 0.1 ug/ul | 0.5 ug/ul | 5 ug/ul | 0.4 ug/ul | 2.5 ug/u/ | 0.8 ug/ul | |
|---|---|---|---|---|---|---|---|
| | E1 | E2 | Ub | 176 | 178 | Hrd1 | Bx12 |
| −E1 (E2 + 176) | — | 0.5 | 0.5 | 1 | — | — | 10 |
| −E2 (E1 + 176) | 1 | — | 0.5 | 1 | — | — | 9.5 |
| −ub (E1 + E2 + 176) | 1 | 0.5 | — | 1 | — | — | 9.5 |
| E1 + E2 + 176 + Ub | 1 | 0.5 | 0.5 | 1 | | — | 9 |
| −E1 (E2 + 178) | — | 0.5 | 0.5 | — | 1 | — | 10 |
| −E2 (E1 + 178) | 1 | — | 0.5 | — | 1 | — | 9.5 |
| −ub (E1 + E2 + 178) | 1 | 0.5 | — | — | 1 | — | 9.5 |
| E1 + E2 + 178 + Ub | 1 | 0.5 | 0.5 | — | 1 | —1 | 9 |
| Hrd1, E1 + E2 + Ub | 1 | 0.5 | 0.5 | — | — | 1 | 8.5 |
| | | | | | | | * |

1. Incubate for 30 minutes at 37° C.
2. Run 12% SDS PAGE gel and transfer to nitrocellulose membrane
3. Incubate with anti-Ubiquitin antibody.

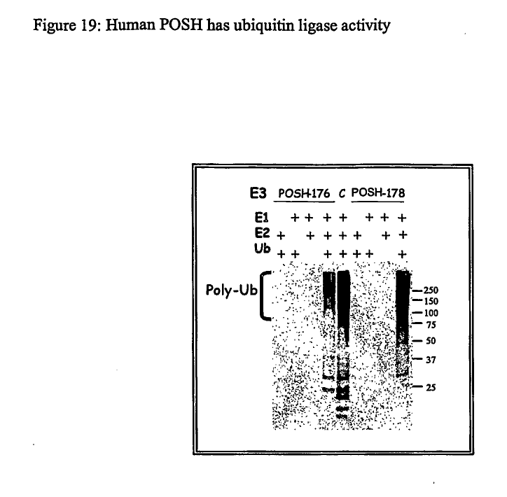

Results, shown in FIG. 19, demonstrate that human POSH has ubiquitin ligase activity.

### Example 5

### POSH Reduction Results in Decreased Secretion of Phospholipase D

Hela SS6 cells (two wells of 6-well plate) were transfected with POSH siRNA or control siRNA (100 nM). 24 hours later each well was split into 5 wells of a 24-well plate. The next day cells were transfected again with 100 nM of either POSH siRNA or control siRNA. The next day cells were washed three times with 1× PBS and than 0.5 ml of PLD incubation buffer (118 mM NaCl, 6 mM KCl, 1 mM CaCl₂, 1.2 mM MgSO4, 12.4 mM HEPES, pH 7.5 and 1% fatty acid free bovine serum albumin) were added.

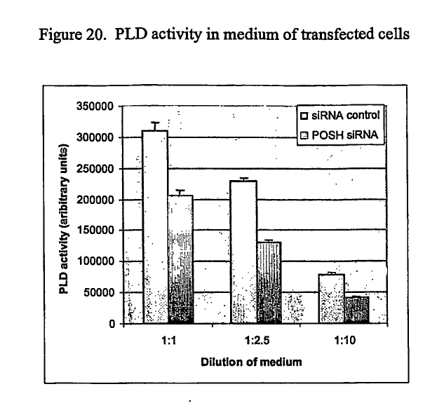

48 hours later medium was collected and centrifuged at 800×g for 15 minutes. The medium was diluted with 5× PLD reaction buffer (Amplex red PLD kit) and assayed for PLD by using the Amplex Red PLD kit (Molecular probes, A-12219). The assay results were quantified and presented below in as a bar graph. The cells were collected and lysed in 1% Triton X-100 lysis buffer (20 mM HEPES-NaOH, pH 7.4, 150 mM NaCl, 1.5 mM MgCl₂, 1 mM EDTA, 1% Triton X-100 and 1× protease inhibitors) for 15 minutes on ice. Lysates were cleared by centrifugation and protein concentration was determined. There were equal protein concentrations between the two transfectants. Equal amount of extracts were immunoprecipitated with anti-POSH antibodies, separated by SDS-PAGE and immunoblotted with anti-POSH antibodies to assess the reduction of POSH levels. There was approximately 40% reduction in POSH levels (FIG. 20).

### Example 6

### Effect of hPOSH on Gag-EGFP Intracellular Distribution

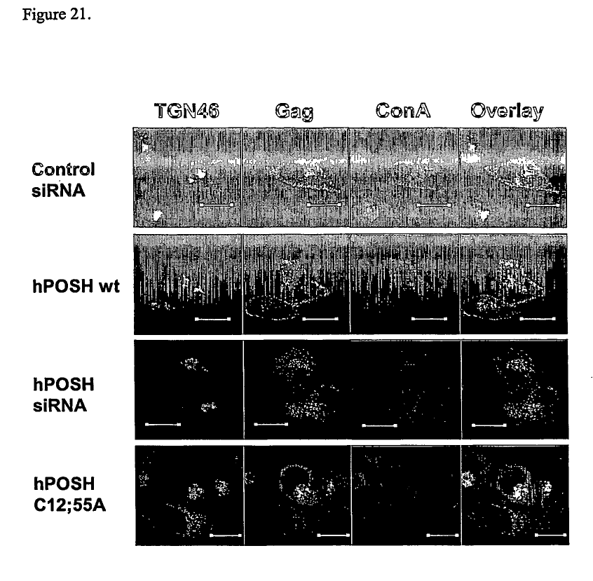

HeLa SS6 were transfected with Gag-EGFP, 24 hours after an initial transfection with either hPOSH-specific or scrambled siRNA (control) (100 nM) or with plasmids encoding either wild type hPOSH or hPOSH C(12,55)A. Fixation and staining was preformed 5 hours after Gag-EGFP transfection. Cells were fixed, stained with Alexa fluor 647-conjugated Concanavalin A (ConA) (Molecular Probes), permeabilized and then stained with sheep anti-human TGN46. After the primary antibody incubation cells were incubated with Rhodamin-conjugated goat anti-sheep. Laser scanning confocal microscopy was performed on LSM510 confocal microscope (Zeiss) equipped with Axiovert 100M inverted microscope using ×40 magnification and 1.3-numerical-aperture oil-immersion lens for imaging. For co-localization experiments, 10 optical horizontal sections with intervals of 1 μm were taken through each preparation (Z-stack). A single median section of each preparation is shown. See FIG. 21.

### Example 7

### POSH-Regulated Intracellular Transport of Myristoylated Proteins

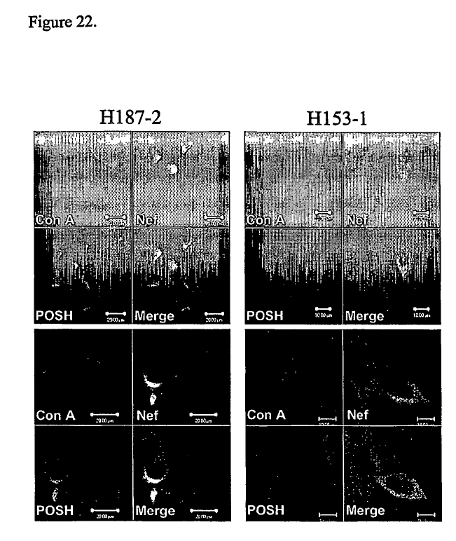

The localization of myristoylated proteins, Gag (see FIG. 21), HIV-1 Nef, Src and Rapsyn, in cells depleted of hPOSH were analyzed by immunofluorescence. In control cells, HIV-1 Nef was found in a perinuclear region co-localized with hPOSH, indicative of a TGN localization (FIG. 22). When hPOSH expression was reduced by siRNA treatment, Nef expression was weaker relative to control and nef lost its TGN, perinuclear localization. Instead it accumulated in punctated intracellular loci segregated from the TGN.

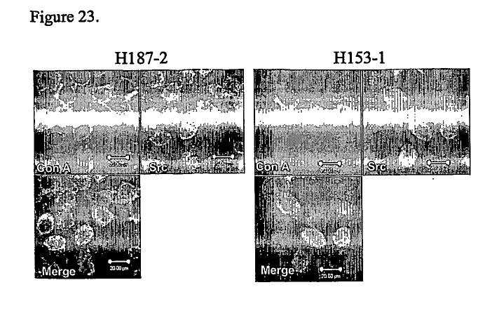

Src is expressed at the plasma membrane and in intracellular vesicles, which are found close to the plasma membrane (FIG. 23, H187 cells). However, when hPOSH levels were reduced, Src was dispersed in the cytoplasm and loses its plasma membrane proximal localization detected in control (H187) cells (FIG. 23, compare H153-1 and H187-2 panels).

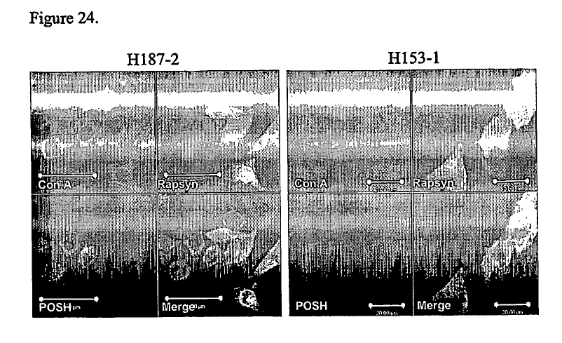

Rapsyn, a peripheral membrane protein expressed in skeletal muscle, plays a critical role in organizing the structure of the nicotinic postsynaptic membrane (Sanes and Lichtman, Annu. Rev. Neurosci. 22: 389442 (1999)). Newly synthesized Rapsyn associates with the TGN and than transported to the plasma membrane (Marchand et al., J. Neurosci. 22: 8891-01 (2002)). In hPOSH-depleted cells (H153-1) Rapsyn was dispersed in the cytoplasm, while in control cells it had a punctuated pattern and plasma membrane localization, indicating that hPOSH influences its intracellular transport (FIG. 24).

Materials and Methods Used:

Antibodies:

Src antibody was purchased from Oncogene research products(Darmstadt, Germany). Nef antibodies were pusrchased from ABI (Columbia, Mass.) and Fitzgerald Industries Interantional (Concord, Mass.). Alexa Fluor conjugated antibodies were pusrchased from Molecular Probes Inc. (Eugene, Oreg.).

hPOSH antibody: Glutathione S-transferase (GST) fusion plasmids were constructed by PCR amplification of hPOSH codons 285-430. The amplified PCR products was cloned into pGEX-6P-2 (Amersham Pharmacia Biotech, Buckinghamshire, UK). The truncated hPOSH protein was generated in E. coli BL21. Bacterial cultures were grown in LB media with carbenicillin (100 μg/ml) and recombinant protein production was induced with 1 mM IPTG for 4 hours at 30° C. Cells were lysed by sonication and the recombinant protein was then isolated from the cleared bacterial lysate by affinity chromatography on a glutathione-sepharose resin (Amersham Pharmacia Biotech, Buckinghamshire, UK). The hPOSH portion of the fusion protein was then released by incubation with PreScission protease (Amersham Pharmacia Biotech, Buckinghamshire, UK) according to the manufacturer's instructions and the GST portion was then removed by a second glutathione-sepharose affinity chromatography. The purified partial hPOSH polypeptide was used to immunize New Zealand white rabbits to generate antibody 15B (Washington Biotechnology, Baltimore, Md.).

Construction of siRNA Retroviral Vectors:

hPOSH scrambled oligonucleotide (5′-CACACACTGCCG TCAACT GTTCAAGAGAC AGTTGACGGCAGTGTGTGTTTTT-3′; and 5′-AATTAAAAAACACA CACTGCCGTCAACTGTC TCTTGAACAGTTGA CGGCAGTGTGTGGGCC-3′) were annealed and cloned into the ApaI-EcoRI digested pSilencer 1.0-US (Ambion) to generate pSIL-scrambled. Subsequently, the U6-promoter and RNAi sequences were digested with BamHI, the ends filled in and the insert cloned into the Olil site in the retroviral vector, pMSVhyg (Clontech), generating pMSCVhyg-U6-scrambled. hPOSH oligonucleotide encoding RNAi against hPOSH (5′-AACAGAGGCCTTGGAAA CCTGGAAGC TTGCAGGTTT CCAAGGCCTCTGTT-3′; and 5′-GATCAACAGAG GCCTTGGAAACCTGC AAGCTTCCAGGTTTCCAA GGCCTCTGTT-3′) were annealed and cloned into the BamHI-EcoRI site of pLIT-U6, generating pLIT-U6 hPOSH-230. pLIT-U6 is an shRNA vector containing the human U6 promoter (amplified by PCR from human genomic DNA with the primers, 5′-GGCCCACTAGTCA AGGTCG GGCA GGAAGA-3′ and 5′-GCCGAATT CAAAAAGGATC CGGCGATATCCGG TGTTTCGTCCTTTCCA-3′) cloned into pLITMUS38 (New England Biolabs) digested with SpeI-EcoRI. Subsequently, the U6 promoter-hPOSH shRNA (pLIT-U6 hPOSH-230 digested with SnaBI and PvuI) was cloned into the Olil site of pMSVhyg (Clontech), generating pMSCVhyg U6-hPOSH-230.

Generation of Stable Clones:

HEK 293T cells were transfected with retroviral RNAi plasmids (pMSCVhyg-U6-POSH-230 and pMSCVhyg-U6-scrambled and with plasmids encoding VSV-G and moloney gag-pol. Two days post transfection, medium containing retroviruses was collected and filtered and polybrene was added to a final concentration of 8 μg/ml. This was used to infect HeLa SS6 cells grown in 60 mm dishes. Forty-eight hours post-infection cells were selected for RNAi expression by the addition of hygromycin to a final concentration of 300 μg/ml. Clones expressing RNAi against hPOSH were named H153, clones expressing scrambled RNAi were named H187.

Transfection and Immunofluorescent Analysis:

Gag-EGFP experiments are described in Example 6 and FIG. 22.

H153 or H187 cells were transfected with Src or Rapsyn-GFP (Image clone image: 3530551 or pNLenv-1). Eighteen hours post transfection cells were washed with PBS and incubated on ice with Alexa Fluor 647 conjugated Con A to label plasma membrane glycoproteins. Subsequently cells were fixed in 3% paraformaldehyde, blocked with PBS containing 4% bovine serum albumin and 1% gelatin. Staining with rabbit anti-Src, rabbit anti-hPOSH (15B) or mouse anti-nef was followed with secondary antibodies as indicated.

Laser scanning confocal microscopy was performed on LSM510 confocal microscope (Zeiss) equipped with Axiovert 100M inverted microscope using ×40 magnification and 1.3-numerical-aperture oil-immersion lens for imaging. For co-localization experiments, 10 optical horizontal sections with intervals of 1 μm were taken through each preparation (Z-stack). A single median section of each preparation is shown.

### Example 8

### POSH Protein-Protein Interactions by Yeast Two Hybrid Assay

POSH-associated proteins were identified by using a yeast two-hybrid assay.

Procedure:

Bait plasmid (GAL4-BD) was transformed into yeast strain AH109 (Clontech) and transformants were selected on defined media lacking tryptophan. Yeast strain Y187 containing pre-transformed Hela cDNA prey (GAL4-AD) library (Clontech) was mated according to the Clontech protocol with bait containing yeast and plated on defined media lacking tryptophan, leucine, histidine and containing 2 mM 3 amino triazol. Colonies that grew on the selective media were tested for beta-galactosidase activity and positive clones were further characterized. Prey clones were identified by amplifying cDNA insert and sequencing using vector derived primers.

Bait:
Plasmid vector: pGBK-T7 (Clontech)
Plasmid name: pPL269-pGBK-T7 GAL4 POSHdR
Protein sequence: Corresponds to aa 53-888 of POSH
(RING domain deleted)
RTLVGSGVEELPSNILLVRLLDGIKQRPWKPGPGGGSGTNCTNALRSQSS
TVANCSSKDLQSSQGGQQPRVQSWSPPVRGIPQLPCAKALYNYEGKEPGD
LKFSKGDIIILRRQVDENWYHGEVNGIHGFFPTNFVQIIKPLPQPPPQCK
ALYDFEVKDKEADKDCLPFAKDDVLTVIRRVDENWAEGMLADKIGIFPIS
YVEFNSAAKQLIEWDKPPVPGVDAGECSSAAAQSSTAPKHSDTKKNTKKR
HSFTSLTMANKSSQASQNRHSMEISPPVLISSSNPTAAARISELSGLSCS
APSQVHISTTGLIVTPPPSSPVTTGPSFTFPSDVPYQAALGTLNPPLPPP
PLLAATVLASTPPGATAAAAAAGMGPRPMAGSTDQIAHLRPQTRPSVYVA
IYPYTPRKEDELELRKGEMFLVFERCQDGWFKGTSMHTSKIGVFPGNYVA
PVTRAVTNASQAKVPMSTAGQTSRGVTMVSPSTAGGPAQKLQGNGVAGSP
SVVPAAVVSAAHIQTSPQAKVLLHMTGQMTVNQARNAVRTVAAHNQERPT
AAVTPIQVQNAAGLSPASVGLSHHSLASPQPAPLMPGSATHTAAISISRA
SAPLACAAAAPLTSPSITSASLEAEPSGRIVTVLPGLPTSPDSASSACGN
SSATKPDKDSKKEKKGLLKLLSGASTKRKPRVSPPASPTLEVELGSAELP
LQGAVGPELPPGGGHGRAGSCPVDGDGPVTTAVAGAALAQDAFHRKASSL
DSAVPIAPPPRQACSSLGPVLNESRPVVCERHRVVVSYPPQSEAELELKE
GDIVFVHKKREDGWFKGTLQRNGKTGLFPGSFVENI

Library Screened: Hela Pretransformed Library (Clontech).

The POSH-AP, HERPUD1 (Hs.146393), was identified by yeast two-hybrid assay. Examples of nucleic acid and amino acid sequences of HERPUD1 are provided below.

Human HERPUD1 mRNA sequence-var1 (public gi:
16507801)
AGAGACGTGAACGGTCGTTGCAGAGATTGCGGGCGGCTGAGACGCCGCCT
GCCTGGCACCTAGGAGCGCAGCGGAGCCCCGACACCGCCGCCGCCGCCAT
GGAGTCCGAGACCGAACCCGAGCCCGTCACGCTCCTGGTGAAGAGCCCCA
ACCAGCGCCACCGCGACTTGGAGCTGAGTGGCGACCGCGGCTGGAGTGTG
GGCCACCTCAAGGCCCACCTGAGCCGCGTCTACCCCGAGCGTCCGCGTCC
AGAGGACCAGAGGTTAATTTATTCTGGGAAGCTGTTGTTGGATCACCAAT
GTCTCAGGGACTTGCTTCCAAAGGAAAAACGGCATGTTTTGCATCTGGTG
TGCAATGTGAAGAGTCCTTCAAAAATGCCAGAAATCAACGCCAAGGTGGC
TGAATCCACAGAGGAGCCTGCTGGTTCTAATCGGGGACAGTATCCTGAGG
ATTCCTCAAGTGATGGTTTAAGGCAAAGGGAAGTTCTTCGGAACCTTTCT
TCCCCTGGATGGGAAAACATCTCAAGGCATCACGTTGGGTGGTTTCCATT
TAGACCGAGGCCGGTTCAGAACTTCCCAAATGATGGTCCTCCTCCTGACG
TTGTAAATCAGGACCCCAACAATAACTTACAGGAAGGCACTGATCCTGAA
ACTGAAGACCCCAACCACCTCCCTCCAGACAGGGATGTACTAGATGGCGA
GCAGACCAGCCCCTCCTTTATGAGCACAGCATGGCTTGTCTTCAAGACTT
TCTTTGCCTCTCTTCTTCCAGAAGGCCCCCCAGCCATCGCAAACTGATGG
TGTTTGTGCTGTAGCTGTTGGAGGCTTTGACAGGAATGGACTGGATCACC
TGACTCCAGCTAGATTGCCTCTCCTGGACATGGCAATGATGAGTTTTTAA
AAAACAGTGTGGATGATGATATGCTTTTGTGAGCAAGCAAAAGCAGAAAC
GTGAAGCCGTGATACAAATTGGTGAACAAAAAATGCCCAAGGCTTCTCAT
GTCTTTATTCTGAAGAGCTTTAATATATACTCTATGTAGTTTAATAAGCA
CTGTACGTAGAAGGCCTTAGGTGTTGCATGTCTATGCTTGAGGAACTTTT
CCAAATGTGTGTGTCTGCATGTGTGTTTGTACATAGAAGTCATAGATGCA
GAAGTGGTTCTGCTGGTACGATTTGATTCCTGTTGGAATGTTTAAATTAC
ACTAAGTGTACTACTTTATATAATCAATGAAATTGCTAGACATGTTTTAG
CAGGACTTTTCTAGGAAAGACTTATGTATAATTGCTTTTTAAAATGCAGT
GCTTTACTTTAAACTAAGGGGAACTTTGCGGAGGTGAAAACCTTTGCTGG
GTTTTCTGTTCAATAAAGTTTTACTATGAATGACCCTGAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AA
Human HERPUD1 mRNA sequence-var2 (public gi:
10441910)
GCTGTGTGGCCCAGGCTTTTCTCAAACTCCTGAGGGCAAGCGATCCTCCC
ACCTCAGCCTCCTGAGTAGCTGGGACTACAGGCATGTGCCACTAGACCTG
GCTCTAAAGACATATATGACACACGAAACCATTTATTTTTCATTTCACAA
TGTTTATTCACATATATGGTATTAGTATTCTAATGTAGTGATGCACTCTA
AATTTGCATTATATTTCCTAGAACATCTGAACAGAGCATAGGAAATTCCC
TATTTTGCCATTATCAGTTCTAACAAAAATCTTAAAAGCACTTTATCATT
TCATTTCCCTGCACTGTAATTTTTTTAAATGATCAAAAACAGTATCATAC
CAAGGCTTACTTATATTGGAATACTATTTTAGAAAGTTGTGGGCTGGGTT
GTATTTATAAATCTTGTTGGTCAGATGTCTGCAATGAGTAAATTTAGCAC
CATTATCAGGAAGCTTTCTCACCAATGACAACTTCATTGGAAGATTTTAA
TGAAAGTGTAGCATACTCTAGGGAAAAATATGAATATTTTTAGCATCTAT
GTATTGAAAATTATGTTGAATAAATGTCAGACTATTTTTTACATAACGTT
GCTTCTGTTTAATTTTGTCACGTTCAGAGGTGGGGGGTAGGAGATGTAAG
CCCTTGACAGCAAAATAATTCCTTTTGCTTGATTTCAGACAGTTGCATCA
GCTCCTTTGTTCTGTGTTCATGTTACACTTATTTAGGTGGCTGAATCCAC
AGAGGAGCCTGCTGGTTCTAATCGGGGACAGTATCCTGAGGATTCCTCAA
GTGATGGTTTAAGGCAAAGGGAAGTTCTTCGGAACCTTTCTTCCCCTGGA
TGGGAAAACATCTCAAGGCCTGAAGCTGCCCAGCAGGCATTCCAAGGCCT
GGGTCCTGGTTTCTCCGGTTACACACCCTATGGGTGGCTTCAGCTTTCCT
GGTTCCAGCAGATATATGCACGACAGTACTACATGCAATATTTAGCAGCC
ACTGCTGCATCAGGGGCTTTTGTTCCACCACCAAGTGCACAAGAGATACC
TGTGGTCTCTGCACCTGCTCCAGCCCCTATTCACAACCAGTTTCCAGCTG
AAAACCAGCCTGCCAATCAGAATGCTGCTCCTCAAGTGGTTGTTAATCCT
GGAGCCAATCAAAATTTGCGGATGAATGCACAAGGTGGCCCTATTGTGGA
AGAAGATGATGAAATAAATCGAGATTGGTTGGATTGGACCTATTCAGCAG
CTACATTTTCTGTTTTTCTCAGTATCCTCTACTTCTACTCCTCCCTGAGC
AGATTCCTCATGGTCATGGGGGCCACCGTTGTTATGTACCTGCATCACGT
TGGGTGGTTTCCATTTAGACCGAGGCCGGTTCAGAACTTCCCAAATGATG
GTCCTCCTCCTGACGTTGTAAATCAGGACCCCAACAATAACTTACAGGAA
GGCACTGATCCTGAAACTGAAGACCCCAACCACCTCCCTCCAGACAGGGA
TGTACTAGATGGCGAGCAGACCAGCCCCTCCTTTATGAGCACAGCATGGC
TTGTCTTCAAGACTTTCTTTGCCTCTCTTCTTCCAGAAGGCCCCCCAGCC
ATCGCAAACTGATGGTGTTTGTGCTGTAGCTGTTGGAGGCTTTGACAGGA
ATGGACTGGATCACCTGACTCCAGCTAGATTGCCTCTCCTGGACATGGCA
ATGATGAGTTTTTAAAAAACAGTGTGGATGATGATATGCTTTTGTGAGCA
AGCAAAAGCAGAAACGTGAAGCCGTGATACAAATTGGTGAACAAAAAATG
CCCAAGGCTTCTCATGTCTTTATTCTGAAGAGCTTTAATATATACTCTAT
GTAGTTTAATAAGCACTGTACGTAGAAGGCCTTAGGTGTTGCATGTCTAT
GCTTGAGGAACTTTTCCAAATGTGTGTGTCTGCATGTGTGTTTGTACATA
GAAGTCATAGATGCAGAAGTGGTTCTGCTGGTACGATTTGATTCCTGTTG
GAATGTTTAAATTACACTAAGTGTACTACTTTATATAATCAATGAAATTG
CTAGACATGTTTTAGCAGGACTTTTCTAGGAAAGACTTATGTATAATTGC
TTTTTAAAATGCAGTGCTTTACTTTAAACTAAGGGGAACTTTGCGGAGGT
GAAAACCTTTGCTGGGTTTTCTGTTCAATAAAGTTTTACTATGAATGACA
AAAAAAAAAAAAAAAAA
Human HERPUD1 mRNA sequence-var3 (public gi:
3005722)
GGCCACCTCAAGGCCCACCTGAGCCGCGTCTACCCCGAGCGTCCGCGTCC
AGAGGACCAGAGGTTAATTTATTCTGGGAAGCTGTTGTTGGATCACCAAT
GTCTCAGGGACTTGCTTCCAAAGGAAAAACGGCATGTTTTGCATCTGGTG
TGCAATGTGAAGAGTCCTTCAAAAATGCCAGAAATCAACGCCAAGGTGGC
TGAATCCACAGAGGAGCCTGCTGGTTCTAATCGGGGACAGTATCCTGAGG
ATTCCTCAAGTGATGGTTTAAGGCAAAGGGAAGTTCTTCGGAACCTTTCT
TCCCCTGGATGGGAAAACATCTCAAGGCCTGAAGCTGCCCAGCAGGCATT
CCAAGGCCTGGGTCCTGGTTTCTCCGGTTACACACCCTATGGGTGGCTTC
AGCTTTCCTGGTTCCAGCAGATATATGCACGACAGTACTACATGCAATAT
TTAGCAGCCACTGCTGCATCAGGGGCTTTTGTTCCACCACCAAGTGCACA
AGAGATACCTGTGGTCTCTGCACCTGCTCCAGCCCCTATTCACAACCAGT
TTCCAGCTGAAAACCAGCCTGCCAATCAGAATGCTGCTCCTCAAGTGGTT
GTTAATCCTGGAGCCAATCAAAATTTGCGGATGAATGCACAAGGTGGCCC
TATTGTGGAAGAAGATGATGAAATAAATCGAGATTGGTTGGATTGGACCT
ATTCAGCAGCTACATTTTCTGTTTTTCTCAGTATCCTCTACTTCTACTCC
TCCCTGAGCAGATTCCTCATGGTCATGGGGGCCACCGTTGTTATGTACCT
GCATCACGTTGGGTGGTTTCCATTTAGACCGAGGCCGGTTCAGAACTTCC
CAAATGATGGTCCTCCTCCTGACGTTGTAAATCAGGACCCCAACAATAAC
TTACAGGAAGGCACTGATCCTGAAACTGAAGACCCCAACCACCTCCCTCC
AGACAGGGATGTACTAGATGGCGAGCAGACCAGCCCCTCCTTTATGAGCA
CAGCATGGCTTGTCTTCAAGACTTTCTTTGCCTCTCTTCTTCCAGAAGGC
CCCCCAGCCATCGCAAACTGATGGTGTTTGTGCTGTAGCTGTTGGAGGCT
TTGACAGGAATGGACTGGATCACCTGACTCCAGCTAGATTGCCTCTCCTG
GACATGGCAATGATGAGTTTTTAAAAAACAGTGTGGATGATGATATGCTT
TTGTGAGCAAGCAAAAGCAGAAACGTGAAGCCGTGATACAAATTGGTGAA
CAAAAAATGCCCAAGGCTTCTCATGTCTTTATTCTGAAGAGCTTTAATAT
ATACTCTATGTAGTTTAATAAGCACTGTACGTAGAAGGCCTTAGGTGTTG
CATGTCTATGCTTGAGGAACTTTTCCAAATGTGTGTGTCTGCATGTGTGT
TTGTACATAGAAGTCATAGATGCAGAAGTGGTTCTGCTGGTACGATTTGA
TTCCTGTTGGAATGTTTAAATTACACTAAGTGTACTACTTTATATAATCA
ATGAAATTGCTAGACATGTTTTAGCAGGACTTTTCTAGGAAAGACTTATG
TATAATTGCTTTTTAAAATGCAGTGCTTTACTTTAAACTAAGGGGAACTT
TGCGGAGGTGAAACCTTTGCTGGGTTTTCTGTTCAATAAAGTTTTACTAT
GAATGACCCTGAAAAAAAAAAAAAAAAAAAAAAA
Human HERPUD1 mRNA sequence-var4 (public gi:
21619176)
CCACGCGTCCGGGTCGTTGCAGAGATTGCGGGCGGCTGAGACGCCGCCTG
CCTGGCACCTAGGAGCGCAGCGGAGCCCCGACACCGCCGCCGCCGCCATG
GAGTCCGAGACCGAACCCGAGCCCGTCACGCTCCTGGTGAAGAGCCCCAA
CCAGCGCCACCGCGACTTGGAGCTGAGTGGCGACCGCGGCTGGAGTGTGG
GCCACCTCAAGGCCCACCTGAGCCGCGTCTACCCCGAGCGTCCGCGTCCA
GAGGACCAGAGGTTAATTTATTCTGGGAATCTGTTGTTGGATCACCAATG
TCTCAGGGACTTGCTTCCAAAGCAGGAAAAACGGCATGTTTTGCATCTGG
TGTGCAATGTGAAGAGTCCTTCAAAAATGCCAGAAATCAACGCCAAGGTG
GCTGAATCCACAGAGGAGCCTGCTGGTTCTAATCGGGGACAGTATCCTGA
GGATTCCTCAAGTGATGGTTTAAGGCAAAGGGAAGTTCTTCGGAACCTTT
CTTCCCCTGGATGGGAAAACATCTCAAGGCCTGAAGCTGCCCAGCAGGCA
TTCCAAGGCCTGGGTCCTGGTTTCTCCGGTTACACACCCTATGGGTGGCT
TCAGCTTTCCTGGTTCCAGCAGATATATGCACGACAGTACTACATGCAAT
ATTTAGCAGCCACTGCTGCATCAGGGGCTTTTGTTCCACCACCAAGTGCA
CAAGAGATACCTGTGGTCTCTGCACCTGCTCCAGCCCCTATTCACAACCA
GTTTCCAGCTGAAAACCAGCCTGCCAATCAGAATGCTGCTCCTCAAGTGG
TTGTTAATCCTGGAGCCAATCAAAATTTGCGGATGAATGCACAAGGTGGC
CCTATTGTGGAAGAAGATGATGAAATAAATCGAGATTGGTTGGATTGGAC
CTATTCAGCAGCTACATTTTCTGTTTTTCTCAGTATCCTCTACTTCTACT
CCTCCCTGAGCAGATTCCTCATGGTCATGGGGGCCACCGTTGTTATGTAC
CTGCATCACGTTGGGTGGTTTCCATTTAGACCGAGGCCGGTTCAGAACTT
CCCAAATGATGGTCCTCCTCCTGACGTTGTAAATCAGGACCCCAACAATA
ACTTACAGGAAGGCACTGATCCTGAAACTGAAGACCCCAACCACCTCCCT
CCAGACAGGGATGTACTAGATGGCGAGCAGACCAGCCCCTCCTTTATGAG
CACAGCATGGCTTGTCTTCAAGACTTTCTTTGCCTCTCTTCTTCCAGAAG
GCCCCCCAGCCATCGCAAACTGATGGTGTTTGTGCTGTAGCTGTTGGAGG
CTTTGACAGGAATGGACTGGATCACCTGACTCCAGCTAGATTGCCTCTCC
TGGACATGGCAATGATGAGTTTTTAAAAAACAGTGTGGATGATGATATGC
TTTTGTGAGCAAGCAAAGCAGAAACGTGAAGCCGTGATACAAATTGGTGA
ACAAAAAATGCCCAAGGCTTCTCATGTCTTTATTCTGAAGAGCTTTAATA
TATACTCTATGTAGTTTAATAAGCACTGTACGTAGAAGGCCTTAGGTGTT
GCATGTCTATGCTTGAGGAACTTTTCCAAATGTGTGTGTCTGCATGTGTG
TTTGTACATAGAAGTCATAGATGCAGAAGTGGTTCTGCTGGTACGATTTG
ATTCCTGTTGGAATGTTTAAATTACACTAAGTGTACTACTTTATATAATC
AATGAAATTGCTAGACATGTTTTAGCAGGACTTTTCTAGGAAAGACTTAT
GTATAATTGCTTTTTAAAATGCAGTGCTTTACTTTAAACTAAGGGGAACT
TTGCGGAGGTGAAAACCTTTGCTGGGTTTTCTGTTCAATAAAGTTTTACT
ATGAATGACCCTGAAAAAAAAAAAAAAA
Human HERPUD1 mRNA sequence-var5 (public gi:
14249882)
AACGGTCGTTGCAGAGATTGCGGGCGGCTGAGACGCCGCCTGCCTGGCAC
CTAGGAGCGCAGCGGAGCCCCGACACCGCCGCCGCCGCCATGGAGTCCGA
GACCGAACCCGAGCCCGTCACGCTCCTGGTGAAGAGCCCCAACCAGCGCC
ACCGCGACTTGGAGCTGAGTGGCGACCGCGGCTGGAGTGTGGGCCACCTC
AAGGCCCACCTGAGCCGCGTCTACCCCGAGCGTCCGCGTCCAGAGGACCA
GAGGTTAATTTATTCTGGGAAGCTGTTGTTGGATCACCAATGTCTCAGGG
ACTTGCTTCCAAAGCAGGAAAAACGGCATGTTTTGCATCTGGTGTGCAAT
GTGAAGAGTCCTTCAAAAATGCCAGAAATCAACGCCAAGGTGGCTGAATC
CACAGAGGAGCCTGCTGGTTCTAATCGGGGACAGTATCCTGAGGATTCCT
CAAGTGATGGTTTAAGGCAAAGGGAAGTTCTTCGGAACCTTTCTTCCCCT
GGATGGGAAAACATCTCAAGGCCTGAAGCTGCCCAGCAGGCATTCCAAGG
CCTGGGTCCTGGTTTCTCCGGTTACACACCCTATGGGTGGCTTCAGCTTT
CCTGGTTCCAGCAGATATATGCACGACAGTACTACATGCAATATTTAGAG
CCACTGCTGCATCAGGCTTTTGTTCCACCACCACCAAGTGCACAAGAGAT
ACCTGTGGTCTCTGCACCTGCTCCAGCCCCTATTCACAACCAGTTTCCAG
CTGAAAACCAGCCTGCCAATCAGAATGCTGCTCCTCAAGTGGTTGTTAAT
CCTGGAGCCAATCAAAATTTGCGGATGAATGCACAAGGTGGCCCTATTGT
GGAAGAAGATGATGAAATAAATCGAGATTGGTTGGATTGGACCTATTCAG
CAGCTACATTTTCTGTTTTTCTCAGTATCCTCTACTTCTACTCCTCCCTG
AGCAGATTCCTCATGGTCATGGGGGCCACCGTTGTTATGTACCTGCATCA
CGTTGGGTGGTTTCCATTTAGACCGAGGCCGGTTCAGAACTTCCCAAATG
ATGGTCCTCCTCCTGACGTTGTAAATCAGGACCCCAACAATAACTTACAG
GAAGGCACTGATCCTGAAACTGAAGACCCCAACCACCTCCCTCCAGACAG
GGATGTACTAGATGGCGAGCAGACCAGCCCCTCCTTTATGAGCACAGCAT
GGCTTGTCTTCAAGACTTTCTTTGCCTCTCTTCTTCCACAAGGCCCCCCA
GCCATCGCAAACTGATGGTGTTTGTGCTGTAGCTGTTGGAGGCTTTGACA
GGAATGGACTGGATCACCTGACTCCAGCTAGATTGCCTCTCCTGGACATG
GCAATGATGAGTTTTTAAAAAACAGTGTGGATGATGATATGCTTTTGTGA
GCAAGCAAAAGCAGAAACGTGAAGCCGTGATACAAATTGGTGAACAAAAA
ATGCCCAAGGCTTCTCATGTCTTTATTCTGAAGAGCTTTAATATATACTC
TATGTAGTTTAATAAGCACTGTACGTAGAAGGCCTTAGGTGTTGCATGTC
TATGCTTGAGGAACTTTTCCAAATGTGTGTGTCTGCATGTGTGTTTGTAC
ATAGAAGTCATAGATGCAGAAGTGGTTCTGCTGGTACGATTTGATTCCTG
TTGGAATGTTTAAATTACACTAAGTGTACTACTTTATATAATCAATGAAA
TTGCTAGACATGTTTTAGCAGGACTTTTCTAGGAAAGACTTATGTATAAT
TGCTTTTTAAAATGCAGTGCTTTACTTTAAACTAAGGGGAACTTTGCGGA
GGTGAAAACCTTTGCTGGGTTTTCTGTTCAATAAAGTTTTACTATGAAAA
AAAAAAAAAAAAAA
Human HERPUD1 mRNA sequence-var6 (public gi:
12652674)
GAACTGTCGTTGCAGAGATTGCGGGCGGCTGAGACGCCGCCTGCCTGGCA
CCTAGGAGCGCAGCGGAGCCCCGACACCGCCGCCGCCGCCATGGAGTCCG
AGACCGAACCCGAGCCCGTCACGCTCCTGGTGAAGAGCCCCAACCAGCGC
CACCGCGACTTGGAGCTGAGTGGCGACCGCGGCTGGAGTGTGGGCCACCT
CAAGGCCCACCTGAGCCGCGTCTACCCCGAGCGTCCGCGTCCAGAGGACC
AGAGGTTAATTTATTCTGGGAAGCTGTTGTTGGATCACCAATGTCTCAGG
GACTTGCTTCCAAAGCAGGAAAAACGGCATGTTTTGCATCTGGTGTGCAA
TGTGAAGAGTCCTTCAAAAATGCCAGAAATCAACGCCAAGGTGGCTGAAT
CCACAGAGGAGCCTGCTGGTTCTAATCGGGGACAGTATCCTGAGGATTCC
TCAAGTGATGGTTTAAGGCAAAGGGAAGTTCTTCGGAACCTTTCTTCCCC
TGGATGGGAAAACATCTCAAGGCCTGAAGCTGCCCAGCAGGCATTCCAAG
GCCTGGGTCCTGGTTTCTCCGGTTACACACCCTATGGGTGGCTTCAGCTT
TCCTGGTTCCAGCAGATATATGCACGACAGTACTACATGCAATATTTAGC
GCCACTGCTGCATCAAGGGGCTTTTGTTCCACCACCAAGTGCACAAGAGA
TACCTGTGGTCTCTGCACCTGCTCCAGCCCCTATTCACAACCAGTTTCCA
GCTGAAAACCAGCCTGCCAATCAGAATGCTGCTCCTCAAGTGGTTGTTAA
TCCTGGAGCCAATCAAAATTTGCGGATGAATGCACAAGGTGGCCCTATTG
TGGAAGAAGATGATGAAATAAATCGAGATTGGTTGGATTGGACCTATTCA
GCAGCTACATTTTCTGTTTTTCTCAGTATCCTCTACTTCTACTCCTCCCT
GAGCAGATTCCTCATGGTCATGGGGGCCACCGTTGTTATGTACCTGCATC
ACGTTGGGTGGTTTCCATTTAGACCGAGGCCGGTTCAGAACTTCCCAAAT
GATGGTCCTCCTCCTGACGTTGTAAATCAGGACCCCAACAATAACTTACA
GGAAGGCACTGATCCTGAAACTGAAGACCCCAACCACCTCCCTCCAGACA
GGGATGTACTAGATGGCGAGCAGACCAGCCCCTCCTTTATGAGCACAGCA
TGGCTTGTCTTCAAGACTTTCTTTGCCTCTCTTCTTCCAGAAGGCCCCCC
AGCCATCGCAAACTGATGGTGTTTGTGCTGTAGCTGTTGGAGGCTTTGAC
AGGAATGGACTGGATCACGTGACTCCAGCTAGATTGCCTCTCCTGGACAT
GGCAATGATGAGTTTTTAAAAAACAGTGTGGATGATGATATGCTTTTGTG
AGCAAGCAAAAGCAGAAACGTGAAGCCGTGATACAAATTGGTGAACAAAA
AATGCCCAAGGCTTCTCATGTCTTTATTCTGAAGAGCTTTAATATATACT
CTATGTAGTTTAATAAGCACTGTACGTAGAAGGCCTTAGGTGTTGCATGT
CTATGCTTGAGGAACTTTTCCAAATGTGTGTGTCTGCATGTGTGTTTGTA
CATAGAAGTCATAGATGCAGAAGTGGTTCTGCTGGTACGATTTGATTCCT
GTTGGAATGTTTAAATTACACTAAGTGTACTACTTTATATAATCAATGAA
ATTGCTAGACATGTTTTAGCAGGACTTTTCTAGGAAAGACTTATGTATAA
TTGCTTTTTAAAATGCAGTGCTTTACTTTAAACTAAGGGGAACTTTGCGG
AGGTGAAAACCTTTGCTGGGTTTTCTGTTCAATAAAGTTTTACTATGAAT
GAAAAAAAAAAAAAAAAAAAA
Human HERPUD1 mRNA sequence-var7 (public gi:
9711684)
AGAGACGTGAACTGTCGTTGCAGAGATTGCGGGCGGCTGAGACGCCGCCT
GCCTGGCACCTAGGAGCGCAGCGGAGCCCCGACACCGCCGCCGCCGCCAT
GGAGTCCGAGACCGAACCCGAGCCGTCACGCTCCTGGTTGAAGAGCCCCA
ACCAGCGCCACCGCGACTTGGAGCTGAGTGGCGACCGCGGCTGGAGTGTG
GGCCACCTCAAGGCCCACCTGAGCCGCGTCTACCCCGAGCGTCCGCGTCC
AGAGGACCAGAGGTTAATTTATTCTGGGAAGCTGTTGTTGGATCACCAAT
GTCTCAGGGACTTGCTTCCAAAGCAGGAAAAACGGCATGTTTTGCATCTG
GTGTGCAATGTGAAGAGTCCTTCAAAAATGCCAGAAATCAACGCCAAGGT
GGCTGAATCCACAGAGGAGCCTGCTGGTTCTAATCGGGGACAGTATCCTG
AGGATTCCTCAAGTGATGGTTTAAGGCAAAGGGAAGTTCTTCGGAACCTT
TCTTCCCCTGGATGGGAAAACATCTCAAGGCCTGAAGCTGCCCAGCAGGC
ATTCCAAGGCCTGGGTCCTGGTTTCTCCGGTTACACACCCTATGGGTGGC
TTCAGCTTTCCTGGTTCCAGCAGATATATGCACGACAGTACTACATGCAA
TATTTAGCAGCCACTGCTGCATCAGGGGCTTTTGTTCCACCACCAAGTGC
ACAAGAGATACCTGTGGTCTCTGCACCTGCTCCAGCCCCTATTCACAACC
AGTTTCCAGCTGAAAACCAGCCTGCCAATCAGAATGCTGCTCCTCAAGTG
GTTGTTAATCCTGGAGCCAATCAAAATTTGCGGATGAATGCACAAGGTGG
CCCTATTGTGGAAGAAGATGATGAAATAAATCGAGATTGGTTGGATTGGA
CCTATTCAGCAGCTACATTTTCTGTTTTTCTCATTATCCTCTACTTCTAC
TCCTCCCTGAGCAGATTCCTCATGGTCATGGGGGCCACCGTTGTTATGTA
CCTGCATCACGTTGGGTGGTTTCCATTTAGACCGAGGCCGGTTCAGAACT
TCCCAAATGATGGTCCTCCTCCTGACGTTGTAAATCAGGACCCCAACAAT
AACTTACAGGAAGGCACTGATCCTGAAACTGAAGACCCCAACCACCTCCC
TCCAGACAGGGATGTACTAGATGGCGAGCAGACCAGCCCCTCCTTTATGA
GCACAGCATGGCTTGTCTTCAAGACTTTCTTTGCCTCTCTTCTTCCAGAA
GGCCCCCCAGCCATCGCAAACTGATGGTGTTTGTGCTGTAGCTGTTGGAG
GCTTTGACAGGAATGGACTGGATCACCTGACTCCAGCTAGATTGCCTCTC
CTGGACATGGCAATGATGAGTTTTTAAAAAACAGTGTGGATGATGATATG
CTTTTGTGAGCAAGCAAAAGCAGAAACGTGAAAGCCGTGATACAAATTGG
TGAACAAAAAATGCCCAAGGTTCTCATGTCTTTATTCTGAAGAGCTTTAA
TATATACTCTATGTAGTTTAATAAGCACTGTACGTAGAAGGCCTTAGGTG
TTGCATGTCTATGCTTGAGGAACTTTTCCAAATGTGTGTGTCTGCATGTG
TGTTTGTACATAGAAGTCATAGATGCAGAAGTGGTTCTGCTGGTACGATT
TGATTCCTGTTGGAATGTTTAAATTACACTAAGTGTACTACTTTATATAA
TCAATGAAATTGCTAGACATGTTTTAGCAGGACTTTTCTAGGAAAGACTT
ATGTATAATTGCTTTTTAAAATGCAGTGCTTTACTTTAAACTAAGGGGAA
CTTTGCGGAGGTGAAAACCTTTGCTGGGTTTTCTGTTCAATAAAGTTTTA
CTATGAATGACCCTG
Human HERPUD1 mRNA sequence-var8 (public gi:
3005718)
GACGTGAACGGTCGTTGCAGAGATTGCGGGCGGCTGAGACGCCGCCTGCC
TGGCACCTAGGAGCGCAGCGGAGCCCCGACACCGCCGCCGCCGCCATGGA
GTCCGAGACCGAACCCGAGCCCGTCACGCTCCTGGTGAAGAGCCCCAACC
AGCGCCACCGCGACTTGGAGCTGAGTGGCGACCGCGGCTGGAGTGTGGGC
CACCTCAAGGCCCACCTGAGCCGCGTCTACCCCGAGCGTCCGCGTCCAGA
GGACCAGAGGTTAATTTATTCTGGGAAGCTGTTGTTGGATCACCAATGTC
TCAGGGACTTGCTTCCAAAGCAGGAAAAACGGCATGTTTTGCATCTGGTG
TGCAATGTGAAGAGTCCTTCAAAAATGCCAGAAATCAACGCCAAGGTGGC
TGAATCCACAGAGGAGCCTGCTGGTTCTAATCGGGGACAGTATCCTGAGG
ATTCCTCAAGTGATGGTTTAAGGCAAAGGGAAGTTCTTCTGAACCTTTCT
TCCCCTGGATGGGAAAACATCTCAAGGCCTGAAGCTGCCCAGCAGGCATT
CCAAGGCCTGGGTCCTGGTTTCTCCGGTTACACACCCTATGGGTGGCTTC
AGCTTTCCTGGTTCCAGCAGATATATGCACGACAGTACTACATGCAATAT
TTAGCAGCCACTGCTGCATCAGGGGCTTTTGTTCCACCACCAAGTGCACA
AGAGATACCTGTGGTCTCTGCACCTGCTCCAGCCCCTATTCACAACCAGT
TTCCAGCTGAAAACCAGCCTGCCAATCAGAATGCTGCTCCTCAAGTGGTT
GTTAATCCTGGAGCCAATCAAAATTTGCGGATGAATGCACAAGGTGGCCC
TATTGTGGAAGAAGATGATGAAATAAATCGAGATTGGTTGGATTGGACCT
ATTCAGCAGCTACATTTTCTGTTTTTCTCAGTATCCTCTACTTCTACTCC
TCCCTGAGCAGATTCCTCATGGTCATGGGGGCCACCGTTGTTATGTACCT
GCATCACGTTGGGTGGTTTCCATTTAGACCGAGGCCGGTTCAGAACTTCC
CAAATGATGGTCCTCCTCCTGACGTTGTAAATCAGGACCCCAACAATAAC
TTACAGGAAGGCACTGATCCTGAAACTGAAGACCCCAACCACCTCCCTCC
AGACAGGGATGTACTAGATGGCGAGCAGACCAGCCCCTCCTTTATGAGCA
CAGCATGGCTTGTCTTCAAGACTTTCTTTGCCTCTCTTCTTCCAGAAGGC
CCCCCAGCCATCGCAAACTGATGGTGTTTGTGCTGTAGCTGTTGGAGGCT
TTGACAGGAATGGACTGGATCACCTGACTCCAGCTAGATTGCCTCTCCTG
GACATGGCAATGATGAGTTTTTAAAAAACAGTGTGGATGATGATATGCTT
TTGTGAGCAAGCAAAAGCAGAAACGTGAAGCCGTGATACAAATTGGTGAA
CAAAAAATGCCCAAGGCTTCTCATGTCTTEATTCTGAAGAGCTTTAATAT
ATACTCTATGTAGTTTAATAAGCACTGTACGTAGAAGGCCTTAGGTGTTG
CATGTCTATGCTTGAGGAACTTTTCCAAATGTGTGTGTCTGCATGTGTGT
TTGTACATAGAAGTCATAGATGCAGAAGTGGTTCTGCTGGTACGATTTGA
TTCCTGTTGGAATGTTTAAATTACACTAAGTGTACTACTTTATATAATCA
ATGAAATTGCTAGACATGTTTTAGCAGGACTTTTCTAGGAAAGACTTATG
TATAATTGCTTTTTAAAATGCAGTGCTTTACTTTAAACTAAGGGGAACTT
TGCGGAGGTGAAAACCTTTGCTGGGTTTTCTGTTCAATAAAGTTTTACTA
TGAATGACCCTGAAAAAAAAAAAAAAAAAAAAAA
Human HERPUD1 mRNA sequence-var9 (public gi:
285960)
CGTGAACGGTCGTTGCAGAGATTGCGGGCGGCTGAGACGCCGCCTGCCTG
GCACCTAGGAGCGCAGCGGAGCCCCGACACCGCCGCCGCCGCCATGGAGT
CCGAGACCGAACCCGAGCCCGTCACGCTCCTGGTGAAGAGCCCCAACCAG
CGCCACCGCGACTTGGAGCTGAGTGGCGACCGCGGCTGGAGTGTGGGCCA
CCTCAAGGCCCACCTGAGCCGCGTCTACCCCGAGCGTCCGCGTCCAGAGG
ACCAGAGGTTAATTTATTCTGGGAAGCTGTTGTTGGATCACCAATGTCTC
AGGGACTTGCTTCCAAAGCAGGAAAAACGGCATGTTTTGCATCTGGTGTG
CAATGTGAAGAGTCCTTCAAAAATGCCAGAAATCAACGCCAAGGTGGCTG
AATCCACAGAGGAGCCTGCTGGTTCTAATCGGGGACAGTATCCTGAGGAT
TCCTCAAGTGATGGTTTAAGGCAAAGGGAAGTTCTTCGGAACCTTTCTTC
CCCTGGATGGGAAAACATCTCAAGGCCTGAAGCTGCCCAGCAGGCATTCC
AAGGCCTGGGTCCTGGTTTCTCCGGTTACACACCCTATGGGTGGCTTCAG
CTTTCCTGGTTCCAGCAGATATATGCACGACAGTACTACATGCAATATTT
AGCAGCCACTGCTGCATCAGGGGCTTTTGTTCCACCACCAAGTGCACAAG
AGATACCTGTGGTCTCTGCACCTGCTCCAGCCCCTATTCACAACCAGTTT
CCAGCTGAAAACCAGCCTGCCAATCAGAATGCTGCTCCTCAAGTGGTTGT
TAATCCTGGAGCCAATCAAAATTTGCGGATGAATGCACAAGGTGGCCCTA
TTGTGGAAGAAGATGATGAAATAAATCGAGATTGGTTGGATTGGACCTAT
TCAGCAGCTACATTTTCTGTTTTTCTCAGTATCCTCTACTTCTACTCCTC
CCTGAGCAGATTCCTCATGGTCATGGGGGCCACCGTTGTTATGTACCTGC
ATCACGTTGGGTGGTTTCCATTTAGACCGAGGCCGGTTCAGAACTTCCCA
AATGATGGTCCTCCTCCTGACGTTGTAAATCAGGACCCCAACAATAACTT
ACAGGAAGGCACTGATCCTGAAACTGAAGACCCCAACCACCTCCCTCCAG
ACAGGGATGTACTAGATGGCGAGCAGACCAGCCCCTCCTTTATGAGCACA
GCATGGCTTGTCTTCAAGACTTTCTTTGCCTCTCTTCTTCCAGAAGGCCC
CCCAGCCATCGCAAACTGATGGTGTTTGTGCTGTAGCTGTTGGAGGCTTT
GACAGGAATGGACTGGATCACCTGACTCCAGCTAGATTGCCTCTCCTGGA
CATGGCAATGATGAGTTTTTAAAAAACAGTGTGGATGATGATATGCTTTT
GTGAGCAAGCAAAAGCAGAAACGTGAAGCCGTGATACAAATTGGTGAACA
AAAAATGCCCAAGGCTTCTCATGTGTTTATTCTGAAGAGCTTTAATATAT
ACTCTATGTAGTTTAATAAGCACTGTACGTAGAAGGCCTTAGGTGTTGCA
TGTCTATGCTTGAGGAACTTTTCCAAATGTGTGTGTCTGCATGTGTGTTT
GTACATAGAAGTCATAGATGCAGAAGTGGTTCTGCTGGTAAGATTTGATT
CCTGTTGGAATGTTTAAATTACACTAAGTGTACTACTTTATATAATCAAT
GAAATTGCTAGACATGTTTTAGCAGGACTTTTCTAGGAAAGACTTATGTA
TAATTGCTTTTTAAAATGCAGTGCTTTACTTTAAACTAAGGGGAACTTTG
CGGAGGTGAAAACCTTTGCTGGGTTTTCTGTTCAATAAAGTTTTACTATG
AATGACCCTG
Human HERPUD1 mRNA sequence-var10 (public gi:
7661869)
GACGTGAACGGTCGTTGCAGAGATTGCGGGCGGCTGAGACGCCGCCTGCC
TGGCACCTAGGAGCGCAGCGGAGCCCCGACACCGCCGCCGCCGCCATGGA
GTCCGAGACCGAACCCGAGCCCGTCACGCTCCTGGTGAAGAGCCCCAACC
AGCGCCACCGCGACTTGGAGCTGAGTGGCGACCGCGGCTGGAGTGTGGGC
CACCTCAAGGCCCACCTGAGCCGCGTCTACCCCGAGCGTCCGCGTCCAGA
GGACCAGAGGTTAATTTATTCTGGGAAGCTGTTGTTGGATCACCAATGTC
TCAGGGACTTGCTTCCAAAGCAGGAAAAACGGCATGTTTTGCATCTGGTG
TGCAATGTGAAGAGTCCTTCAAAAATGCCAGAAATCAACGCCAAGGTGGC
TGAATCCACAGAGGAGCCTGCTGGTTCTAATCGGGGACAGTATCCTGAGG
ATTCCTCAAGTGATGGTTTAAGGCAAAGGGAAGTTCTTCGGAACCTTTCT
TCCCCTGGATGGGAAAACATCTCAAGGCCTGAAGCTGCCCAGCAGGCATT
CCAAGGCCTGGGTCCTGGTTTCTCCGGTTACACACCCTATGGGTGGCTTC
AGCTTTCCTGGTTCCAGCAGATATATGCACGACAGTACTACATGCAATAT
TTAGCAGCCACTGCTGCATCAGGGGCTTTTGTTCCACCACCAAGTGCACA
AGAGATACCTGTGGTCTCTGCACCTGCTCCAGCCCCTATTCACAACCAGT
TTCCAGCTGAAAACCAGCCTGCCAATCAGAATGCTGCTCCTCAAGTGGTT
GTTAATCCTGGAGCCAATCAAAATTTGCGGATGAATGCACAAGGTGGCCC
TATTGTGGAAGAAGATGATGAAATAAATCGAGATTCCTTGGATTGGACCT
ATTCAGCAGCTACATTTTCTGTTTTTCTCAGTATCCTCTACTTCTACTCC
TCCCTGAGCAGATTCCTCATGGTCATGGGGGCCACCGTTGTTATGTACCT
GCATCACGTTGGGTGGTTTCCATTTAGACCGAGGCCGGTTCAGAACTTCC
CAAATGATGGTCCTCCTCCTGACGTTGTAAATCAGGACCCCAACAATAAC
TTACAGGAAGGCACTGATCCTGAAACTGAAGACCCCAACCACCTCCCTCC
AGACAGGGATGTACTAGATGGCGAGCAGACCAGCCCCTCCTTTATGAGCA
CAGCATGGCTTGTCTTCAAGACTTTCTTTGCCTCTCTTCTTCCAGAAGGC
CCCCCAGCCATCGCAAACTGATGGTGTTTGTGCTGTAGCTGTTGGAGGCT
TTGACAGGAATGGACTGGATCACCTGACTCCAGCTAGATTGCCTCTCCTG
GACATGGCAATGATGAGTTTTTAAAAAACAGTGTGGATGATGATATGCTT
TTGTGAGCAAGCAAAAGCAGAAACGTGAAGCCGTGATACAAATTGGTGAA
CAAAAAATGCCCAAGGCTTCTCATGTCTTTATTCTGAAGAGCTTTAATAT
ATACTCTATGTAGTTTAATAAGCACTGTACGTAGAAGGCCTTAGGTGTTG
CATGTCTATGCTTGAGGAACTTTTCCAAATGTGTGTGTCTGCATGTGTGT
TTGTACATAGAAGTCATAGATGCAGAAGTGGTTCTGCTGGTACGATTTGA
TTCCTGTTGGAATGTTTAAATTACACTAAGTGTACTACTTTATATAATCA
ATGAAATTGCTAGACATGTTTTAGCAGGACTTTTCTAGGAAAGACTTATG
TATAATTGCTTTTTAAAATGCAGTGCTTTACTTTAAACTAAGGGGAACTT
TGCGGAGGTGAAAACCTTTGCTGGGTTTTCTGTTCAATAAAGTTTTACTA
TGAATGACCCTGAAAAAAAAAAAAAAAAAAAAAA
Human HERPUD1 Protein sequence-var1 (public gi:
16507802)
MESETEPEPVTLLVKSPNQRHRDLELSGDRGWSVGHLKAHLSRVYPERPR
PEDQRLILSGKLLLDHQCLRDLLPKEKRHVLHLVCNVKSPSKMPEINAKV
AESTEEPAGSNRGQYPEDSSSDGLRQREVLRNLSSPGWENISRHHVGWFP
FRPRPVQNFPNDGPPPDVVNQDPNNNLQEGTDPETEDPNHLPPDRDVLDG
EQTSPSFMSTAWLVFKTFFASLLPEGPPAIAN
Human HERPUD1 Protein sequence-var2 (public gi:
10441911)
MQYLAATAASGAFVPPPSAQEIPVVSAPAPAPIHNQFPAENQPANQNAAP
QVVVNPGANQNLRMNAQGGPIVEEDDEINRDWLDWTYSAATFSVFLSILY
FYSSLSRFLMVMGATVVMYLHHVGWFPFRPRPVQNFPNDGPPPDVVNQDP
NNNLQEGTDPETEDPNHLPPDRDVLDGEQTSPSFMSTAWLVFKTFFASLL
PEGPPAIAN
Human HERPUD1 Protein sequence-var3 (public gi:
3005723)
GHLKAHLSRVYPERPRPEDQRLIYSGKLLLDHQCLRDLLPKEKRHVLHLV
CNVKSPSKMPEINAKVAESTEEPAGSNRGQYPEDSSSDGLRQREVLRNLS
SPGWENISRPEAAQQAFQGLGPGFSGYTPYGWLQLSWFQQIYARQYYMQY
LAATAASGAFVPPPSAQEIPVVSAPAPAPIHNQFPAENQPANQNAAPQVV
VNPGANQNLRMNAQGGPIVEEDDEINRDWLDWTYSAATFSVFLSILYFYS
SLSRFLMVMGATVVMYLHHVGWFPFRPRPVQNFPNDGPPPDVVNQDPNNN
LQEGTDPETEDPNHLPPDRDVLDGEQTSPSFMSTAWLVFKTFFASLLPEG
PPAIAN
Human HERPUD1 Protein sequence-var4 (public gi:
7661870)
MESETEPEPVTLLVKSPNQRHRDLELSGDRGWSVGHLKAHLSRVYPERPR
PEDQRLIYSGKLLLDHQCLRDLLPKQEKRHVLHLVCNVKSPSKMPEINAK
VAESTEEPAGSNRGQYPEDSSSDGLRQREVLRNLSSPGWENISRPEAAQQ
AFQGLGPGFSGYTPYGWLQLSWFQQIYARQYYMQYLAATAASGAFVPPPS
AQEIPVVSAPAPAPIHNQFPAENQPANQNAAPQVVVNPGANQNLRMNAQG
GPIVEEDDEINRDWLDWTYSAATFSVFLSILYFYSSLSRFLMVMGATVVM
YLHHVGWFPFRPRPVQNFPNDGPPPDVVDQDPNNNLQEGTDPETEDPNHL
PPDRDVLDGEQTSPSFMSTAWLVFKTFFASLLPEGPPAIAN
Rat HERPUD1 mRNA sequence (public gi: 16758961)
AAGACACCAAGTGTCGTTGTGGGGTCGCAGACGGCTGCGTCGCCGCCCGT
TCGGCATCCCTGAGCGCAGTCGAGCCTCCAGCGCCGCAGACATGGAGCCC
GAGCCACAGCCCGAGCCGGTCACGCTGCTGGTGAAGAGCCCCAATCAGCG
CCACCGCGACTTGGAGCTGAGTGGCGACCGCGGTTGGAGTGTGAGTCGCC
TCAAGGCCCACCTGAGCCGAGTCTACCCCGAACGCCCGCGCCCAGAGGAC
CAGAGGTTAATTTATTCTGGGAAGCTGCTGTTGGATCACCAATGTCTCCA
AGACTTGCTTCCAAAGCAGGAAAAGCGACATGTTTTGCACCTCGTGTGCA
ATGTGAGGAGTCCCTCAAAAAAGCCAGAAGCCAGCACAAAGGGTGCTGAG
TCCACAGAGCAGCCGGACAACACTAGTCAGGCACAGTATCCTGGGGATTC
CTCAAGCGATGGCTTACGGGAAAGGGAAGTCCTTCGGAACCTTCCTCCCT
CTGGATGGGAGAACGTCTCTAGGCCTGAAGCCGTCCAGCAGACTTTCCAA
GGCCTCGGGCCCGGCTTCTCTGGCTACACCACCTACGGGTGGCTGCAGCT
CTCCTGGTTCCAGCAGATCTATGCAAGACAGTACTACATGCAATACTTGG
CTGCCACTGCTGCTTCAGGAGCTTTTGGCCCTACACCAAGTGCACAAGAA
ATACCTGTGGTCTCTACACCGGCTCCCGCCCCTATACACAACCAGTTTCC
GGCAGAAAACCAGCCGGCCAATCAGAATGCAGCCGCTCAAGCGGTTGTTA
ATCCCGGAGCCAATCAGAACTTGCGGATGAATGCACAAGGCGGCCCTCTG
GTGGAAGAAGATGATGAGATAAACCGAGACTGGTTGGATTGGACCTACTC
AGCAGCGACATTTTCCGTTTTCCTCAGCATTCTTTACTTCTACTCCTCCC
TGAGCAGATTCCTCATGGTCATGGGCGCCACCGTAGTCATGTACCTGCAC
CACGTCGGGTGGTTTCCATTCAGACAGAGGCCAGTTCAGAACTTCCCAGA
TGACGGTCCCCCTCAGGAAGCTGCCAACCAGGACCCCAACAATAACCTCC
AGGGAGGTTTGGACCCTGAAATGGAAGACCCCAACCGCCTCCCCGTAGGC
CGTGAAGTGCTGGACCCTGAGCATACCAGCCCCTCGTTCATGAGCACAGC
ATGGCTAGTCTTCAAGACTTTCTTTGCCTCTCTTCTTCCGGAAGGCCCAC
CAGCCCTAGCAAACTGATGGCCCCTGTGCTCTGTTGCTGGAGGCTTTCAC
AGCTTGGACTGGATCGTCCCCTGGCGTGGACTCGAGAGAGTCATTGAAAA
CCCACAGGATGACGATGTGCTTCTGTGCCAAGCAAAAGCACAAACTAAGA
CATGAAGCCGTGGTACAAACTGAACAGGGCCCCTCATGTCGTTATTCTGA
AGAGCTTTAATGTATACTGTATGTAGTCTCATAGGCACTGTAAACAGAAG
GCCCAGGGTCGCATGTTCTGCCTGAGCACCTCCCCAGACGTGTGTGCATG
TGTGCCGTACATGGAAGTCATAGACGTGTGTGCATGTGTGCTCTACATGG
AAGTCATAGATGCAGAAACGGTTCTGCTGGTTCGATTTGATTCCTGTTGG
AATGTTGCAATTACACTAAGTGTACTACTTTATATAATCAGTGACTTGCT
AGACATGTTAGCAGGACTTTTCTAGGAGAGACTTATTGTATCATTGCTTT
TTAAAACGCAGTGCTTACTTACTGAGGGCGGCGACTTGGCACAGGTAAAG
CCTTTGCCGGGTTTTCTGTTCATAAAGTTTTGCTATGAACGACAAAAAAA
AAAAAAA
Rat HERPUD1 Protein sequence (public gi: 16758962)
MEPEPQPEPVTLLVKSPNQRHRDLELSGDRGWSVSRLKAHLSRVYPERPR
PEDQRLIYSGKLLLDHQCLQDLLPKQEKRHVLHLVCNVRSPSKKPEASTK
GAESTEQPDNTSQAQYPGDSSSDGLREREVLRNLPPSGWENVSRPEAVQQ
TFQGLGPGFSGYTTYGWLQLSWFQQIYARQYYMQYLAATAASGAFGPTPS
AQEIPVVSTPAPAPIHNQFPAENQPANQNAAAQAVVNPGANQNLRMNAQG
GPLVEEDDEINRDWLDWTYSAATFSVFLSILYFYSSLSRFLMVMGATVVM
YLHHVGWFPFRQRPVQNFPDDGPPQEAANQDPNNNLQGGLDPEMEDPNRL
PVGREVLDPEHTSPSFMSTAWLVFKTFFASLLPEGPPALAN
Mouse HERPUD1 mRNA sequence (public gi: 11612514)
AAAGACGCCAAGTGTCGTTGTGTGGTCTCACACGGCTGCGTCGCCGCCCG
TTCGGCATCCCTGAGCGCAGTCGAGCCGCCAGCGACGCAGACATGGAGCC
CGAGCCACAGCCCGAGCCGGTCACGCTGCTGGTGAAGAGTCCCAATCAGC
GCCACCGCGACTTGGAGCTGAGTGGCGACCGCAGTTGGAGTGTGAGTCGC
CTCAAGGCCCACCTGAGCCGAGTCTACCCCGAGCGCCCGCGTCCAGAGGA
CCAGAGGTTAATTTATTCTGGGAAGCTGCTGTTGGATCACCAGTGTCTCC
AAGATTTGCTTCCAAAGCAGGAAAAGCGACATGTTTTGCACCTTGTGTGC
AATGTGAAGAATCCCTCCAAAATGCCAGAAACCAGCACAAAGGGTGCTGA
ATCCACAGAGCAGCCGGACAACTCTAATCAGACACAGCATCCTGGGGACT
CCTCAAGTGATGGTTTACGGCAAAGAGAAGTTCTTCGGAACCTTTCTCCC
TCCGGATGGGAGAACATCTCTAGGCCTGAGGCTGTCCAGCAGACTTTCCA
AGGCCTGGGGCCTGGCTTCTCTGGCTACACAACGTATGGGTGGCTGCAGC
TCTCCTGGTTCCAGCAGATCTATGCAAGGCAGTACTACATGCAATACTTA
GCTGCCACTGCTGCATCAGGAACTTTTGTCCCGACACCAAGTGCACAAGA
GATACCTGTGGTCTCTACACCTGCTCCGGCTCCTATACACAACCAGTTTC
CGGCAGAAAACCAGCCGGCCAATCAGAATGCAGCTGCTCAAGCGGTTGTC
AATCCCGGAGCCAATCAGAACTTGCGGATGAATGCACAAGGTGGCCCCCT
GGTGGAGGAAGATGATGAGATAAACCGAGACTGGTTGGATTGGACCTATT
CCGCAGCGACGTTTTCTGTTTTCCTCAGCATCCTTTACTTCTACTCGTCG
CTGAGCAGATTTCTCATGGTCATGGGTGCCACTGTAGTCATGTACCTGCA
CCACGTCGGGTGGTTTCCGTTCAGACAGAGGCCAGTTCAGAACTTCCCGG
ATGATGGTGGTCCTCGAGATGCTGCCAACCAGGACCCCAACAATAACCTC
CAGGGAGGTATGGACCCAGAAATGGAAGACCCCAACCGCCTCCCCCCAGA
CCGCGAAGTGCTGGACCCTGACCACACCAGCCCCTCGTTTATGAGCACAG
CATGGCTAGTCTTCAAGACTTTCTTTGCCTCTCTTCTTCCAGAAGGCCCA
CCAGCCCTAGCCAACTGATGGCCCTTGTGCTCTGTCGCTGGTGGCTTTGA
CAGCTCGGACTGGATCGTCTGGCTCCGGCTCCTTTTCCTCCCCTGGCGTG
GACTCGACAGAGTCATTGAAAACCCACAGGATGACATGTGCTTCTGTGCC
AAGCAAAAGCACAAACTAAGACATGAAGCCGTGGTACAAACTGAACAGGG
CCCCTCATGTCGTTATTCTGAAGAGCTTTAATGTATACTGTATGTAGTTT
CATAGGCACTGTAAGCAGAAGGCCCAGGGTCGCATGTTCTGCCTGAGCAC
CTCCCCAGATGTGTGTGCATGTGTGCTGTACATGGAAGTCATAGACGTGT
GTGCATGTGTGCTCTACATGGAAGTCATAGATGCAGAAACGGTTCTGCTG
GTTCGATTTGATTCCTGTTGGAATGTTCAAATTACACTAAGTGTACTACT
TTATATAATCAGTGAATTGCTAGACATGTTAGCAGGACTTTTCTAGGAGA
GACTTATGTATAATTGCTTTTTAAAATGCAGTGCTTTCCTTTAAACCGAG
GGTGGCGACTTGGCAGAGGTAAAACCTTGCCGAGTTTTCTGTTCAATAAA
GTTTTGCTATGAATGACTGT
Mouse HERPUD1 Protein sequence (public gi:
11612515)
MEPEPQPEPVTLLVKSPNQRHRDLELSGDRSWSVSRLKAHLSRVYPERPR
PEDQRLIYSGKLLLDHQCLQDLLPKQEKRHVLHLVCNVKNPSKMPETSTK
GAESTEQPDNSNQTQHPGDSSSDGLRQREVLRNLSPSGWENISRPEAVQQ
TFQGLGPGFSGYTTYGWLQLSWFQQIYARQYYMQYLAATAASGTFVPTPS
AQEIPVVSTPAPAPIHNQFPAENQPANQNAAAQAVVNPGANQNLRMNAQG
GPLVEEDDEINRDWLDWTYSAATFSVFLSILYFYSSLSRFLMVMGATVVM
YLHHVGWFPFRQRPVQNFPDDGGPRDAANQDPNNNLQGGMDPEMEDPNRL
PPDREVLDPEHTSPSFMSTAWLVFKTFFASLLPEGPPALAN

### Example 9

### HERPUD1 Depletion by siRNA Reduces HIV Maturation

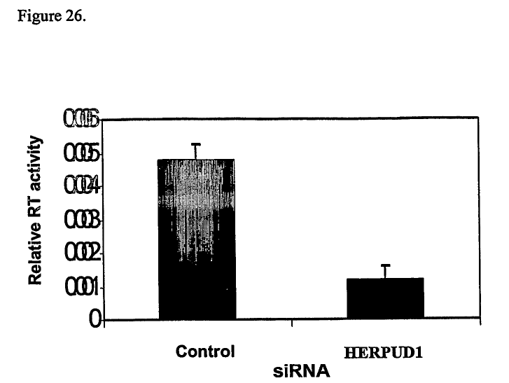

Hela SS6 cells were transfeted with siRNA directed against HERPUD1 and with a plsmid encoding HIV proviral genome (pNLenv-1). Twenty four hours post-HIV transfection, virus-like particles (VLP) secreted into the medium were isolated and reverse transcriptase activity was determined. HIV release of active RT is an indication for a release of processed and mature virus. When the levels of HERPUD1 were reduced RT activity was inhibited by 80%, demonstrating the importance of HERPUD1 in HIV-maturation. See FIG. 26.

Experimental Outline

Cell Culture and Transfection:

HeLa SS6 were kindly provided by Dr. Thomas Tuschl (the laboratory of RNA Molecular Biology, Rockefeller University, New York, N.Y.). Cells were grown in Dulbecco's modified Eagle's medium (DMEM) supplemented with 10% heat-inactivated fetal calf serum and 100 U/ml penicillin and 100 μg/ml streptomycin. For transfections, HeLa SS6 cells were grown to 50% confluency in DMEM containing 10% FCS without antibiotics. Cells were then transfected with the relevant double-stranded siRNA (50-100 nM) (HERPUD1: 5′-GGGAAGUUCUUCGGAACCUdTdT-3′ and 5′-dTdTCCCUUCAAGAAGCCUUGGA-5′) using lipofectamin 2000 (Invitrogen, Paisley, UK). A day following the initial transfection cells were split 1:3 in complete medium and co-transfected 24 hours later with HIV-INLenv1 (2 μg per 6-well) (Schubert et al., J. Virol. 72:2280-88 (1998)) and a second portion of double-stranded siRNA.

Assay for Virus Release

Virus and virus-like particle (VLP) release was determined one day after transfection with the proviral DNA as previously described (Adachi et al., J. Virol. 59: 284-91 (1986); Fukumori et al., Vpr. Microbes Infect. 2: 1011-17 (2000); Lenardo et al., J. Virol. 76: 5082-93 (2002)). The culture medium of virus-expressing cells was collected and centrifuged at 500×g for 10 minutes. The resulting supernatant was passed through a 0.45 μm-pore filter and the filtrate was centrifuged at 14,000×g for 2 hours at 4° C. The resulting supernatant was removed and the viral-pellet was re-suspended in SDS-PAGE sample buffer. The corresponding cells were washed three times with phosphate-buffered saline (PBS) and then solubilized by incubation on ice for 15 minutes in lysis buffer containing the following components: 50 mM HEPES-NaOH, (pH 7.5), 150 mM NaCl, 1.5 mM MgCl₂, 0.5% NP-40, 0.5% sodium deoxycholate, 1 mM EDTA, 1 mM EGTA and 1:200 dilution of protease inhibitor cocktail (Calbiochem, La Jolla, Calif.). The cell detergent extract was then centrifuged for 15 minutes at 14,000×g at 4° C. The VLP sample and a sample of the cleared extract (normally 1:10 of the initial sample) were resolved on a 12.5% SDS-polyacrylamide gel, then transferred onto nitrocellulose paper and subjected to immunoblot analysis with rabbit anti-CA antibodies. The CA was detected either after incubation with a secondary anti-rabbit horseradish peroxidase-conjugated antibody and detected by Enhanced Chemi-Luminescence (ECL) (Amersham Pharmacia) or after incubation with a secondary anti-rabbit antibody conjugated to Cy5 (Jackson Laboratories, West Grove, Pa.) and detected by fluorescence imaging (Typhoon instrument, Molecular Dynamics, Sunnyvale, Calif.). The Pr55 and CA were then quantified by densitometry and the amount of released VLP was then determined by calculating the ratio between VLP-associated CA and intracellular CA and Pr55 as previously described (Schubert et al., J. Virol. 72:2280-88 (1998)).

Analysis of Reverse Transcriptase Activity in Supernatants

RT activity was determined in pelleted VLP (see above) by using an RT assay kit (Roche, Germany; Cat. No. 1468120). Briefly, VLP pellets were resuspended in 40 μl RT assay lysis buffer and incubated at room temperature for 30 minutes. At the end of incubation 20 μl RT assay reaction mix was added to each sample and incubation continued at 37° C. overnight. Samples (60 μl) were than transferred to MTP strip wells and incubated at 37° C. for 1 hour. Wells were washed five times with wash buffer and DIG-POD added for a one-hour incubation at 37° C. At the end of incubation wells were washed five times with wash buffer and ABST substrate solution was added and incubated until color developed. The absorbance was read in an ELISA reader at 405 nm (reference wavelength 492 nm). The resulting signal intensity is directly proportional to RT activity; RT concentration was determined by plotting against a known amount of RT enzyme included in separate wells of the reaction.

### Example 10

### POSH-Depleted Cells Have Lower Levels of Herp and It is Not Monoubiquitinated

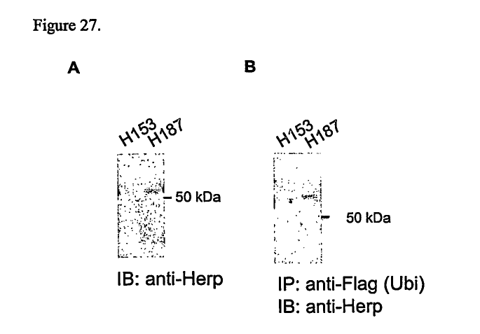

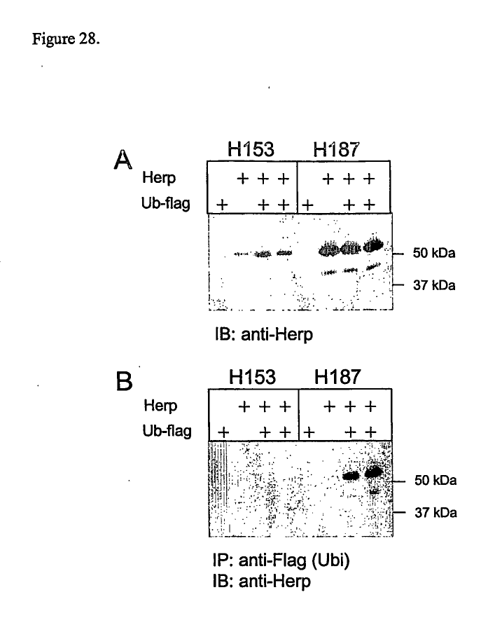

POSH-depleted cells and their control counterparts were lysed and immunoblotted with anti-herp antibodies. Cells depleted of POSH (H153 RNAi stables cell lines) cells have lower levels of Herp compared with control cells (H187 RNAi) (FIG. 27 panel A). When cells were transfected with a plasmid encoding flagged-tagged ubiquitin, and immunoprecipitated with anti-flag antibodies to immunoprecipitate ubiquitinated proteins, Herp was ubiquitinated only in H187 cells and not in H153 cells (FIG. 27 panel B). When the aforementioned cells were transfected with Herp-encoding plasmid, exogenous herp levels were also reduced in H153 cells compared to H187 cells (FIG. 28 panel A) and the ubiquitination of exogenous herp was reduced in the former cells, similar to endogenous Herp. The molecular weight of ubiquitinated Herp is as predicated to full-length Herp and does not seem as a high molecular weight smear, a characteristic of polyubiquitinated proteins. Thus POSH is responsible for the mono-ubiquitination of Herp, and in the absence of this modification herp is subjected to degradation, which may be mediated by the proteosome.

Materials and Methods

Plasmid Generation

Full-length Herp was cloned from image clone MGC:45131 IMAGE:5575914 (GeneBank Accesion BC032673) into pCMV-SPORT6.

Antibody Production

Herp1 (amino acids 1 to 251) was amplified from a plasmid (3Gd4) obtained by yeast two hybrid screen for interactors of POSH. The amplified open reading frame was cloned into pGEX-6P, expressed in E. coli BL21 by induction with 1 mM IPTG and purified on glutathione-agarose. The purified protein was cleaved with Precision™ protease (Amersham Biosciences) and the GST moiety removed by glutathione chromatography. The protein was injected into rabbits (Washington Biotechnology) to produce anti-Herp1 sera.

Transfections and Antibody Detection

Twenty-four hours prior to transfection POSH-RNAi clones (H153) or control-RNAi clones (H187) cells were plated in 10 cm dishes in growth medium (DMEM containing 10% fetal calf serun without antibiotics). Cells were transfected with lipofectamin 2000 (Invitrogen Corporation) and either Herp-expression plasmid (2.5 μg) or empty vector (2.5 μg) and a vector encoding Flag-tagged ubiquitin (1 μg). Twenty-four hours post-trasnfection cells were lysed in lysis buffer (50 mM Tris-HCl, pH7.6, 1.5 mM MgCl2, 150 mM NaCL, 10% glycerol, 1 mM EDTA, 1 mM EGTA, 0.5% NP40 and 0.5% sodium deoxycholate, containing protease inhibitors) and subjected to immunoprecipitation with anti-Flag antibodies (Sigma, F7425) to precipitate ubiquitinated proteins. Immunoprecipitated material and total cell lysates were separated on 10% SDS-PAGE and transferred to nitrocellulose membranes which were immunoblotted with anti-Herp antibodies.

Generation of H187 and H153 Cell Lines

To relieve the necessity for multiple transfections and to improve the reproducibility of hPOSH reduction, we have generated two cell lines, H187 and H153 constitutively expressing an integrated control and hPOSH siRNA (respectively).

Construction of shRNA retroviral vectors—hPOSH scrambled oligonucleotide (5′-CACACACTGCCGTCAACTGTTCAAGAGACAGTTGACGGCAGTGTGTGTTT TTT-3′; and 5′-AATTAAAAAACACACACTGCCGTCAACTGTCTCTTGAACA GTTGACGGCAGTGTGTGGGCC-3′) were annealed and cloned into the ApaI-EcoRI digested pSilencer 1.0-U6 (Ambion, Inc.) to generate pSIL-scrambled. Subsequently, the U6-promoter and RNAi sequences were digested with BamHI, and blunted by end filling. The insert was cloned into the Olil site in the retroviral vector, pMSCVhyg (13D Biosciences Clontech), generating pMSCVhyg-U6-scrambled. The hPOSH oligonucleotide encoding RNAi against hPOSH (5′-AACAGAGGCCTTGGAAACCTGGAAGCTTGCAGGTTTCCAAGGCCTCT GTT-3′; and 5 ′-GATCAACAGAGGCCTTGGAAACCTGCAAGCTTCCAGGTTTCCAAGGC CTCTGTT-3′) were annealed and cloned into the BamHI-EcoRV site of pLIT-U6, generating pLIT-U6 hPOSH-230. The pLIT-U6 is an shRNA vector containing the human U6 promoter (amplified by PCR from human genomic DNA with the primers, 5′-GGCCCACTAGTCAAGGTCGGGCAGGAAGA-3′ and 5′-GCCGAATTCAAAAAGGATCCGGCGATATCCGGTGTTTCGTCCTTTCCA-3′) cloned into pLITMUS38 (New England Biolabs, Inc.) digested with SpeI-EcoRI. Subsequently, the U6 promoter-hPOSH shRNA (pLIT-U6 hPOSH-230 digested with SnaBI and PvuI) was cloned into the Olil site of pMSCVhyg (BD Biosciences Clontech) generating pMSCVhyg U6-hPOSH-230.

Recombinant retrovirus production—HEK 293T cells were transfected with retroviral RNAi plasmids (pMSCVhyg-U6-POSH-230 and pMSCVhyg-U6-scrambled and with plasmids encoding VSV-G and Moloney Gag-pol. Two days post-transfection, the retrovirus-containing medium was collected and filtered.

Infection and selection—Polybrene (Hexadimethrine bromide) (Sigma) (8μg/ml) was added to the filtered and the treated medium was subsequently used to infect HeLa SS6 cells. Forty-eight hours post-infection clones were selected for RNAi expression by the addition of hygromycin (300 μg/ml). Clones expressing the scrambled and the hPOSH RNAi were termed H187 and H153 (respectively).

### Example 11

### Amyloid Precursor Protein Levels are Reduced in Cells that Have Reduced Levels of POSH

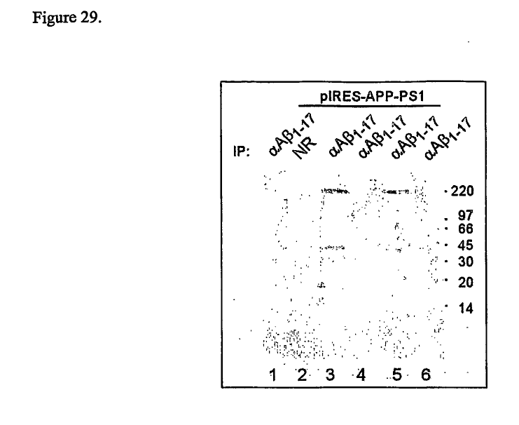

HeLa SS6 cells that express reduced levels of POSH (H153) and control cells expressing scrambled RNAi (H187) were transfected with a plasmid expressing amyloid precursor protein (APP) and presenilin 1 (PS1). Cells were metabolic labeled and protein extracts were immunoprecipitated with anti-amyloid beta specific antibody, which recognize an epitope common to APP, C199 and Aβ polypeptides. A labeled protein was specifically precipitated by the antibody in H187-transfected cells (see FIG. 29, Lanes 3 and 5). However, this polypeptide was not recognized in H153 cells (see FIG. 29, Lanes 4 and 6) indicating that APP steady state levels are reduced in H153 and may be rapidly degraded in these cells.

Methods

Cloning of pIRES-APP-PS1

Cloning was performed in two steps: Presenilin 1 (PS1) was first cloned from human brain library into pIREs (pIREs-PS1). Then APP-695 was obtained from amplifying two image clones (3639599 and 5582406) and mixing their PCR products in an additional PCR reaction to yield full-length APP695 that was further ligated into pIREs-PS1 to generate pIREs-APP-PS1.

Transfection, Metabolic Labeling and Immunoisolation of Amyloid Beta (Aβ)

Hela SS6 cells expressing POSH-specific RNAi or scrambled RNAi (H153 and H187, respectively) were transfected with pIREs-APP-PS1 (24 μg) using lipofectamin 2000 reagent (Invitrogen, LTD). Twenty-four hours post-transfection, cells were metabolic labeled with 1 mCi of ³⁵S-methionine at 37° C. for an additional twenty-four hours. Media was collected from cells and spun at 3000 rpm for 10 min to pellet cell debris. Protease inhibitors and 2 mM 1,10-phenanthroline were added to the cleared cell media. Cells were lysed in lysis buffer (50 mM Tris-HCl, pH7.8, 150 mM sodium chloride, 1 mM EDTA, 0.5% NP-40, 0.5% sodium deoxycholate and protease inhibitors). Cell media and lysate were immunoprecipitated with anti-Aβ(1-17) antibody (6E10) (Chemicon) or a non-relevant (NR) antibody. Precipitated proteins were separated on 16% Tris-Tricine gel. Gel was dried and bands detected by phosphoimager (Typhoon instrument, Amersham Biosciences, Corp.).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the subject applications have been discussed, the above specification is illustrative and not restrictive. Many variations of the applications will become apparent to those skilled in the art upon review of this specification and the claims below. The full scope of the applications should be determined by reference to the claims, along with their full scope of equivalents, and the specification, along with such variations.

1. An isolated, purified or recombinant complex comprising a POSH polypeptide and a POSH-associated protein (POSH-AP).

2. The complex of claim 1, wherein the POSH-AP is HERPUD1.

3. The complex of claim 2, wherein the HERPUD1 is ubiquitinated.

4. The complex of claim 2, wherein the HERPUD1 is monoubiquitinated.

5-15. (canceled)

16. A method of identifying an agent that inhibits a neurological disorder, comprising:
a) forming a mixture comprising a POSH polypeptide, a POSH-AP, ubiquitin and a test agent; and
b) detecting ubiquitination of the POSH-AP,
wherein an agent that inhibits ubiquitination of the POSH-AP is an agent that inhibits a neurological disorder.

17. The method of claim 16, wherein the POSH-AP is HERPUD1.

18. The method of claim 16, further comprising testing the effect of the agent on POSH-mediated ubiquitination of a second substrate.

19. The method of claim 18, wherein the second substrate is POSH.

20. (canceled)

21. The method of claim 16, wherein the agent inhibits POSH-mediated ubiquitination of HERPUD1.

22. The method of claim 21, wherein the agent does not substantially inhibit POSH auto-ubiquitination.

23-25. (canceled)

26. The method of claim 16, wherein the neurological disorder is selected from among: Alzheimer's disease, Parkinson's disease, Huntington's disease, Pick's disease, Niemann-Pick's disease, prion-associated diseases, depression, and schizophrenia.

27. (canceled)

28. The method of claim 16, wherein said agent is selected from among: an siRNA construct, a small molecule, an antibody, and an antisense construct.

29. (canceled)

30. A method of identifying an agent to treat a neurological disorder, the method comprising identifying a test agent that disrupts a complex of claim 2.

31. The method of claim 30, wherein the neurological disorder is selected from among: Alzheimer's disease, Parkinson's disease, Huntington's disease, Pick's disease, Niemann-Pick's disease, prion-associated diseases, depression, and schizophrenia.

32-34. (canceled)

35. A method of testing an agent for use in treatment of a neurological disorder, comprising contacting cells that produce amyloid polypeptide with an agent that inhibits POSH activity and/or expression.

36. The method of claim 35, wherein the agent inhibits POSH ubiquitin ligase activity.

37. The method of claim 36, wherein the agent inhibits the ubiquitination of HERPUD1.

38. The method of claim 35, wherein the agent inhibits the expression of POSH.

39. The method of claim 35, wherein the agent is selected from among: an siRNA construct, a small molecule, an antibody, and an antisense construct.

40. The method of claim 35, further comprising evaluating the effect of the agent on apoptosis in the cell.


SEQUENCE LISTING (70 sequences)

SEQ ID NO: 1 - DNA, 2667 bp, Homo sapiens
atggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatga

SEQ ID NO: 2 - protein, 888 aa, Homo sapiens
MDESALLDLLECPVCLERLDASAKVLPCQHTFCKRCLLGIVGSRNELRCPECRTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 3 - DNA, 5128 bp, Homo sapiens
ctgagagacactgcgagcggcgagcgcggtggggccgcatctgcatcagccgccgcagccgctgcggggccgcgaacaaagaggaggagccgaggcgcgagagcaaagtctgaaatggatgttacatgagtcattttaagggatgcacacaactatgaacatttctgaagattttttctcagtaaagtagataaagatggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatgaggagactgacactgaagaagcttaaaatcacttcacacaacaaagtagcacaaagcagtttaacagaaagagcacatttgtggacttccagatggtcaggagatgagcaaaggattggtatgtgactctgatgccccagcacagttaccccagcgagcagagtgaagaagatgtttgtgtgggttttgttagtctggattcggatgtataaggtgtgccttgtactgtctgatttactacacagagaaacttttttttttttttaagatatatgactaaaatggacaattgtttacaaggcttaactaatttatttgcttttttaaacttgaacttttcgtataatagatacgttctttggattatgattttaagaaattattaatttatgaaatgataggtaaggagaagctggattatctcctgttgagagcaagagattcgttttgacatagagtgaatgcattttcccctctcctcctccctgctaccattatattttggggttatgttttgcttctttaagatagaaatcccagttctctaatttggttttcttctttgggaaaccaaacatacaaatgaatcagtatcaattagggcctggggtagagagacagaaacttgagagaagagaagttagtgattccctctctttctagtttggtaggaatcaccctgaagacctagtcctcaatttaattgtgtgggtttttaattttcctagaatgaagtgactgaaacaatgagaaagaatacagcacaacccttgaacaaaatgtatttagaaatatatttagttttatagcagaagcagctcaattgtttggttggaaagtaggggaaattgaagttgtagtcactgtctgagaatggctatgaagcgtcatttcacattttaccccaactgacctgcatgcccaggacacaagtaaaacatttgtgagatagtggtggtaagtgatgcactcgtgttaagtcaaaggctataagaaacactgtgaaaagttcatattcatccattgtgattctttccccacgtcttgcatgtattactggattcccacagtaatatagactgtgcatggtgtgtatatttcattgcgatttcctgttaagatgagtttgtactcagaattgaccaattcaggaggtgtaaaaataaacagtgttctcttctctaccccaaagccactactgaccaaggtctcttcagtgcactcgctccctctctggctaaggcatgcattagccactacacaagtcattagtgaaagtggtcttttatgtcctcccagcagacagacatcaaggatgagttaaccaggagactactcctgtgactgtggagctctggaaggcttggtgggagtgaatttgcccacaccttacaattgtggcaggatccagaagagcctgtctttttatatccattccttgatgtcattggcctctcccaccgatttcattacggtgccacgcagtcatggatctgggtagtccggaaaacaaaaggagggaagacagcctggtaatgaataagatccttaccacagttttctcatgggaaatacataataaaccctttcatctttttttttttcctttaagaattaaaactgggaaatagaaacatgaactgaaaagtcttgcaatgacaagaggtttcatggtcttaaaaagatactttatatggttgaagatgaaatcattcctaaattaaccttttttttaaaaaaaaacaatgtatattatgttcctgtgtgttgaatttaaaaaaaaaaaatactttacttggatattcatgtaatatataaaggtttggtgaaatgaactttagttaggaaaaagctggcatcagctttcatctgtgtaagttgacaccaatgtgtcataatattctttattttgggaaattagtgtattttataaaaattttaaaaagaaaaaagactactacaggttaagataatttttttacctgtcttttctccatattttaagctatgtgattgaagtacctctgttcatagtttcctggtataaagttggttaaaatttcatctgttaatagatcattaggtaatataatgtatgggttttctattggttttttgcagacagtagagggagattttgtaacaagggcttgttacacagtgatatggtaatgataaaattgcaatttatcactccttttcatgttaataatttgaggactggataaaaggtttcaagattaaaatttgatgttcaaacctttgt

SEQ ID NO: 4 - DNA, 2331 bp, Homo sapiens
ctgagagacactgcgagcggcgagcgcggtggggccgcatctgcatcagccgccgcagccgctgcggggccgcgaacaaagaggaggagccgaggcgcgagagcaaagtctgaaatggatgttacatgagtcattttaaggatgcacacaactatgaacatttctgaagattttttctcagtaaagtagataaagatggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagc

SEQ ID NO: 5 - protein, 712 aa, Homo sapiens
MDESALLDLLECPVCLERLDASAKVLPCQHTFCKRCLLGIVGSRNELRCPECRTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDS

SEQ ID NO: 6 - DNA, 4182 bp, Homo sapiens
atttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgctgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatgaggagactgacactgaagaagcttaaaatcacttcacacaacaaagtagcacaaagcagtttaacagaaagagcacatttgtggacttccagatggtcaggagatgagcaaaggattggtatgtgactctgatgccccagcacagttaccccagcgagcagagtgaagaagatgtttgtgtgggttttgttagtctggattcggatgtataaggtgtgccttgtactgtctgatttactacacagagaaacttttttttttttttaagatatatgactaaaatggacaattgtttacaaggcttaactaatttatttgcttttttaaacttgaacttttcgtataatagatacgttctttggattatgattttaagaaattattaatttatgaaatgataggtaaggagaagctggattatctcctgttgagagcaagagattcgttttgacatagagtgaatgcattttcccctctcctcctccctgctaccattatattttggggttatgttttgcttctttaagatagaaatcccagttctctaatttggttttcttctttgggaaaccaaacatacaaatgaatcagtatcaattagggcctggggtagagagacagaaacttgagagaagagaagttagtgattccctctctttctagtttggtaggaatcaccctgaagacctagtcctcaatttaattgtgtgggtttttaattttcctagaatgaagtgactgaaacaatgagaaagaatacagcacaacccttgaacaaaatgtatttagaaatatatttagttttatagcagaagcagctcaattgtttggttggaaagtaggggaaattgaagttgtagtcactgtctgagaatggctatgaagcgtcatttcacattttaccccaactgacctgcatgcccaggacacaagtaaaacatttgtgagatagtggtggtaagtgatgcactcgtgttaagtcaaaggctataagaaacactgtgaaaagttcatattcatccattgtgattctttccccacgtcttgcatgtattactggattcccacagtaatatagactgtgcatggtgtgtatatttcattgcgatttcctgttaagatgagtttgtactcagaattgaccaattcaggaggtgtaaaaataaacagtgttctcttctctaccccaaagccactactgaccaaggtctcttcagtgcactcgctccctctctggctaaggcatgcattagccactacacaagtcattagtgaaagtggtcttttatgtcctcccagcagacagacatcaaggatgagttaaccaggagactactcctgtgactgtggagctctggaaggcttggtgggagtgaatttgcccacaccttacaattgtggcaggatccagaagagcctgtctttttatatccattccttgatgtcattggcctctcccaccgatttcattacggtgccacgcagtcatggatctgggtagtccggaaaacaaaaggagggaagacagcctggtaatgaataagatccttaccacagttttctcatgggaaatacataataaaccctttcatctttttttttttcctttaagaattaaaactgggaaatagaaacatgaactgaaaagtcttgcaatgacaagaggtttcatggtcttaaaaagatactttatatggttgaagatgaaatcattcctaaattaaccttttttttaaaaaaaaacaatgtatattatgttcctgtgtgttgaatttaaaaaaaaaaaatactttacttggatattcatgtaatatataaaggtttggtgaaatgaactttagttaggaaaaagctggcatcagctttcatctgtgtaagttgacaccaatgtgtcataatattctttattttgggaaattagtgtattttataaaaattttaaaaagaaaaaagactactacaggttaagataatttttttacctgtcttttctccatattttaagctatgtgattgaagtacctctgttcatagtttcctggtataaagttggttaaaatttcatctgttaatagatcattaggtaatataatgtatgggttttctattggttttttgcagacagtagagggagattttgtaacaagggcttgttacacagtgatatggtaatgataaaattgcaatttatcactccttttcatgttaataatttgaggactggataaaaggtttcaagattaaaatttgatgttcaaacctttgt

SEQ ID NO: 7 - protein, 638 aa, Homo sapiens
ISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 8 - DNA, 3206 bp, Mouse
gggcagcgggctcggcggggctgcatctaccagcgctgcggggccgcgaacaaaggcgagcagcggaggcgcgagagcaaagtctgaaatggatgttacatgaatcactttaagggctgcgcacaactatgaacgttctgaagccgttttctcactaaagtcactcaagatggatgagtctgccttgttggaccttctggagtgccctgtgtgtctagaacgcctggatgcttccgcaaaggtcttaccctgccagcataccttttgcaaacgctgtttgctggggattgtgggttcccggaatgaactcagatgtcccgaatgccggactcttgttggctctggggtcgacgagctccccagtaacatcctactggtcagacttctggatggcatcaagcagaggccttggaaacccggccctggtgggggcggcgggaccacctgcacaaacacattaagggcgcagggcagcactgtggttaattgtggctcgaaagatctgcagagctcccagtgtggacagcagcctcgggtgcaagcctggagccccccagtgaggggaatacctcagttaccgtgtgccaaagcattatataactacgaaggaaaagagcccggagaccttaagttcagcaaaggcgacaccatcattctgcgccgacaggtggatgagaattggtaccacggggaagtcagcggggtccacggctttttccccactaacttcgtgcagatcatcaaacctttacctcagcccccgcctcagtgcaaagcactttacgactttgaagtgaaagacaaggaagctgacaaagattgccttcccttcgcaaaggacgacgtactgaccgtgatccgcagagtggatgaaaactgggctgaaggaatgctggcagataaaataggaatatttccaatttcatacgtggagtttaactcagctgccaagcagctgatagagtgggataagcctcccgtgccaggagtggacacggcagaatgcccctcagcgacggcgcagagcacctctgcctcaaagcaccccgacaccaagaagaacaccaggaagcgacactccttcacctccctcaccatggccaacaagtcttcccaggggtcccagaaccgccactccatggagatcagccctcctgtgctcatcagttccagcaaccccacagccgcagcccgcatcagcgaactgtccgggctctcctgcagcgccccgtctcaggtccatataagcaccactgggttaattgtgaccccaccccctagcagcccggtgacaactggccctgcgttcacgttcccttcagatgtcccctaccaagctgcccttggaagtatgaatcctccacttcccccaccccctctcctggcggccaccgtactcgcctccaccccgtcaggcgctactgctgctgttgctgctgctgctgccgccgccgccgctgctggaatgggacccaggcctgtgatggggtcctctgaacagattgcacatttacggcctcagactcgtcccagtgtatatgttgctatatatccgtacactccccggaaggaagacgaactggagctgaggaaaggggagatgtttttggtgtttgagcgttgccaggacggctggtacaaagggacatcgatgcataccagcaagataggcgttttccctggcaactatgtggcgcccgtcacaagggcggtgacgaatgcctcccaagctaaagtctctatgtctactgcgggtcaggcaagtcgcggggtgaccatggtcagcccttccactgcaggaggacctacacagaagccccaaggaaacggcgtggccggaaatcccagcgtcgtccccacggctgtggtgtcagcagctcatatccagacaagtcctcaggctaaggtcctgctgcacatgtctgggcagatgacagtcaatcaggcccgcaatgctgtgaggacagttgcagcacatagccaggaacgccccacagcagcagtgactcccatccaggtccagaatgccgcctgccttggtcctgcatccgtgggcctgccccatcattctctggcctcccaacctctgcctccaatggcgggtcctgctgcccacggtgctgccgtcagcatcagtcgaaccaatgcccccatggcctgcgctgcaggggcttctctggcctccccaaatatgaccagtgccatgttggagacagagcccagtggtcgcacagtgaccatcctccctggactccccacatctccagagagtgctgcatcagcgtgtgggaacagttcagctgggaaaccagacaaggacagtaagaaagaaaaaaagggcctactgaagctgctttctggtgcctccaccaaacgcaagccccgagtctcccctccagcatcacctaccctggatgtggagctgggtgctggggaggctcccttgcagggagcagtaggtcctgagctgccgctagggggcagccacggcagagtggggtcatgccccacagatggtgatggtccagtggccgctggaacagcagccctagcccaggatgccttccaccgcaagacaagctccctggactccgcagtgcccattgctccaccacctcgccaggcctgctcctccctgggcccagtcatgaatgaggcccggcctgttgtttgtgaaaggcacagggtggtggtttcctaccctcctcagagtgaggccgaacttgaactcaaggaaggagatattgtgtttgttcataagaaacgagaggacggctggttcaaaggcacgttacagaggaatgggaagactggccttttcccagggagctttgtggaaaacatctgagaagacgggacacggagaaagcttatcatcacaccacgtgtgactaaagagcacaaagcagtttcatagaaagagcacatctgtggacttccagatcttcaagaaccgagcagaagatgggcacctgactccagagccccggcctggttaccccaggggcagagggaaggaggacacacctgtgtgggttccgtctctctgggttctgatgtgtaaagtgtgccttgtaatgtctaatggactttacagataaatgtctttttttttttaagatgtataactaaaatggacaattgtttacaaggcttaactaatttatttgcttttttaaaacttgaactttcttgtaatagcaaat

SEQ ID NO: 9 - protein, 892 aa, Mouse
MDESALLDLLECPVCLERLDASAKVLPCQHTFCKRCLLGIVGSRNELRCPECRTLVGSGVDELPSNILLVRLLDGIKQRPWKPGPGGGGGTTCTNTLRAQGSTVVNCGSKDLQSSQCGQQPRVQAWSPPVRGIPQLPCAKALYNYEGKEPGDLKFSKGDTIILRRQVDENWYHGEVSGVHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDTAECPSATAQSTSASKHPDTKKNTRKRHSFTSLTMANKSSQGSQNRHSMEISPPVLISSSNPTAAARISELSGLSCSAPSQVHISTTGLIVTPPPSSPVTTGPAFTFPSDVPYQAALGSMNPPLPPPPLLAATVLASTPSGATAAVAAAAAAAAAAGMGPRPVMGSSEQIAHLRPQTRPSVYVAIYPYTPRKEDELELRKGEMFLVFERCQDGWYKGTSMHTSKIGVFPGNYVAPVTRAVTNASQAKVSMSTAGQASRGVTMVSPSTAGGPTQKPQGNGVAGNPSVVPTAVVSAAHIQTSPQAKVLLHMSGQMTVNQARNAVRTVAAHSQERPTAAVTPIQVQNAACLGPASVGLPHHSLASQPLPPMAGPAAHGAAVSISRTNAPMACAAGASLASPNMTSAMLETEPSGRTVTILPGLPTSPESAASACGNSSAGKPDKDSKKEKKGLLKLLSGASTKRKPRVSPPASPTLDVELGAGEAPLQGAVGPELPLGGSHGRVGSCPTDGDGPVAAGTAALAQDAFHRKTSSLDSAVPIAPPPRQACSSLGPVMNEARPVVCERHRVVVSYPPQSEAELELKEGDIVFVHKKREDGWFKGTLQRNGKTGLFPGSFVENI

SEQ ID NO: 10 - DNA, 3149 bp, Drosophila melanogaster
catttgtatccgcttggccacgagctttggctgcacttggcaaacttaataaattaaacattgaatcctgcctattgcaacgataatataatctgatttagtgcattaagaacgacaagtagcgattataatagtagattttagcatttgagctaaatttatttcccaaccgcgtcttgggattgcgtatgcgtgagccagtacctgcatgtgtgtgtgttttggaatgtggccctgcacgaaattcaaatagtgaccatccttgagattttgcatactggcaagatggacgagcacacgttaaacgacctgttggagtgctccgtgtgtcttgagcgactggacaccacatcgaaggtgctgccatgccagcacaccttctgccgcaaatgcttgcaggacattgtggccagtcagcacaagttgcgatgcccggagtgccgcatcctggtctcttgcaaaattgatgagctgcctccaaacgtcttgctgatgcgaatcttagaaggcatgaaacaaaatgcagcagctggcaaaggagaagaaaagggagaggagactgaaacacagccggaaagggccaaacctcagccgccagcggaatcagtggccccgcctgacaaccaactactccagctgcagtcacatcagcaatctcatcagccggctcgtcacaagcaacgtcgatttctactcccccacgcctatgccctctttgacttcgcctccggtgaagccaccgatctaaagttcaagaaaggggatctgatactgatcaagcatcgcatcgacaacaactggtttgtgggtcaagcgaatggtcaggagggcacatttcccatcaactacgtcaaggtatcggttccgctgcccatgccgcagtgcattgccatgtatgactttaagatggggcccaacgacgaggagggatgcctcgaatttaagaaaagcactgtaatacaggtaatgcgccgagttgatcataattgggcagaaggacgaattggccagaccatcggaatctttccaatagcattcgttgagctgaatgcagcggccaaaaagctgttggacagcgggctacacacccatccattctgccatccaccgaagcaacaggggcagcgggcccttcctccggttccagttattgatcccacggtggtcacggaatccagttcgggatcctccaattccacgccgggcagcagcaattcaagctccacatccagctcgaataactgcagtccgaatcaccaaatctcactgccgaataccccccaacatgtagtagcttccggatcggcgtctgttcgtttccgtgacaagggagcaaaggagaaacgccactcactaaatgctttgctgggaggaggagctccattaagtctgctgcagaccaaccgccattcggctgaaattcttagcctgccccatgaactaagccgcttggaagtttccagctcaacagctctaaaacccacgtcagccccacagacatcgcgtgtacttaagaccactgttcagcagcagatgcaaccgaatttaccctggggatacttagccctgttcccatacaaaccacgccaaacggatgagctggaattaaaaaagggttgtgtttacattgtgaccgaacgatgtgtggacggttggttcaagggaaaaaactggttggacatcactggagtgttcccgggcaactacctgacgcccctgcgcgcccgcgaccagcagcagttaatgcatcaatggaaatatgttccccaaaatgcagacgcccagatggcacaagtacagcagcatccagttgcaccagatgtgcgactcaacaacatgctgtccatgcaaccgcctgatttgccacctcgtcagcagcaggctaccgccacgaccaccagttgctctgtgtggtcgaaaccagtggaggcgctgttcagcagaaaatcggagcccaagcctgaaactgccacagcttcgactacgagcagcagttcctctggagcagtgggacttatgaggagattaactcacatgaaaacacgctccaaatctccgggagcgtccttgcagcaagttccgaaagaagctattagcacaaatgtggaatttacaacaaacccatcagctaaattgcatccagtacatgtaagatccggctcgtgccccagtcagctgcagcacagtcaaccgctcaatgaaactccagcagccaagacagcggcacaacaacagcagttcctacccaagcagctgccttccgcttctacgaacagcgtttcgtacggatcgcaacgcgtgaaaggaagcaaggaacgtcctcacttgatttgcgcgagacaatcattagatgcagctacatttcgcagtatgtacaacaatgccgcgtcgccgccgccacctactacttccgtggccccagctgtctacgccggcggtcagcaacaggtgattcctggaggtggagcgcaatcccagttgcatgccaatatgattattgcacccagccatcggaagtcgcacagcctagatgcgagtcatgtgctgagtcccagcagcaatatgatcacggaggcggccattaaggccagcgccaccactaagtctccttactgcacgagggaaagtcgattccgctgcattgtgccgtatccaccaaacagtgacattgaactagagctacatttgggcgacattatctacgtccagcggaagcagaagaacggctggtataagggcacccatgcccgtacccacaaaaccgggctgttccccgcctcctttgttgaaccggattgttaggaaagttatggttcaaactagaatttattaagcgaaattccaaattacttgtctaaaaggattcaatcgtcggtctattcgggcttccaaatacgcaatctcatatttctcttttcaaaaaagaaaccgttttgtactcttccaatcgaatgggcagctcgccgttgtacttttttatacaatgcttgatcaaaataggctagccatgtaagacttagggaacagttacttaagccttagcgattagttagctagagaaataatctaaccgatccttgtgccctctacaaagttatttgtaatatacgatactcagtaataaaaaaaaaaaaaaaaaaaaaaaaaaa

SEQ ID NO: 11 - protein, 838 aa, Drosophila melanogaster
MDEHTLNDLLECSVCLERLDTTSKVLPCQHTFCRKCLQDIVASQHKLRCPECRILVSCKIDELPPNVLLMRILEGMKQNAAAGKGEEKGEETETQPERAKPQPPAESVAPPDNQLLQLQSHQQSHQPARHKQRRFLLPHAYALFDFASGEATDLKFKKGDLILIKHRIDNNWFVGQANGQEGTFPINYVKVSVPLPMPQCIAMYDFKMGPNDEEGCLEFKKSTVIQVMRRVDHNWAEGRIGQTIGIFPIAFVELNAAAKKLLDSGLHTHPFCHPPKQQGQRALPPVPVIDPTVVTESSSGSSNSTPGSSNSSSTSSSNNCSPNHQISLPNTPQHVVASGSASVRFRDKGAKEKRHSLNALLGGGAPLSLLQTNRHSAEILSLPHELSRLEVSSSTALKPTSAPQTSRVLKTTVQQQMQPNLPWGYLALFPYKPRQTDELELKKGCVYIVTERCVDGWFKGKNWLDITGVFPGNYLTPLRARDQQQLMHQWKYVPQNADAQMAQVQQHPVAPDVRLNNMLSMQPPDLPPRQQQATATTTSCSVWSKPVEALFSRKSEPKPETATASTTSSSSSGAVGLMRRLTHMKTRSKSPGASLQQVPKEAISTNVEFTTNPSAKLHPVHVRSGSCPSQLQHSQPLNETPAAKTAAQQQQFLPKQLPSASTNSVSYGSQRVKGSKERPHLICARQSLDAATFRSMYNNAASPPPPTTSVAPAVYAGGQQQVIPGGGAQSQLHANMIIAPSHRKSHSLDASHVLSPSSNMITEAAIKASATTKSPYCTRESRFRCIVPYPPNSDIELELHLGDIIYVQRKQKNGWYKGTHARTHKTGLFPASFVEPDC

SEQ ID NO: 12 - DNA, 18 bp, Artificial Sequence
  primer
cttgccttgccagcatac

SEQ ID NO: 13 - DNA, 18 bp, Artificial Sequence
  primer
ctgccagcattccttcag

SEQ ID NO: 14 - DNA, 21 bp, Artificial Sequence
  target sequence
aacagaggccttggaaacctg

SEQ ID NO: 15 - DNA, 21 bp, Artificial Sequence
  siRNA
ttcagaggccuuggaaaccug

SEQ ID NO: 16 - DNA, 21 bp, Artificial Sequence
  siRNA
ttcagguuuccaaggccucug

SEQ ID NO: 17 - DNA, 21 bp, Artificial Sequence
  target sequence
aaagagcctggagaccttaaa

SEQ ID NO: 18 - DNA, 21 bp, Artificial Sequence
  siRNA
ttagagccuggagaccuuaaa

SEQ ID NO: 19 - DNA, 21 bp, Artificial Sequence
  siRNA
ttuuuaaggucuccaggcucu

SEQ ID NO: 20 - DNA, 21 bp, Artificial Sequence
  target sequence
aaggattggtatgtgactctg

SEQ ID NO: 21 - DNA, 21 bp, Artificial Sequence
  siRNA
ttggauugguaugugacucug

SEQ ID NO: 22 - DNA, 21 bp, Artificial Sequence
  siRNA
ttcagagucacauaccaaucc

SEQ ID NO: 23 - DNA, 21 bp, Artificial Sequence
  target sequence
aagctggattatctcctgttg

SEQ ID NO: 24 - DNA, 21 bp, Artificial Sequence
  siRNA
ttgcuggauuaucuccuguug

SEQ ID NO: 25 - DNA, 21 bp, Artificial Sequence
  siRNA
ttcaacaggagauaauccagc

SEQ ID NO: 26 - protein, 41 aa, Artificial Sequence
  RING domain
CPVCLERLDASAKVLPCQHTFCKRCLLGIVGSRNELRCPEC

SEQ ID NO: 27 - protein, 56 aa, Artificial Sequence
  SH3 domain
PCAKALYNYEGKEPGDLKFSKGDIIILRRQVDENWYHGEVNGIHGFFPTNFVQIIK

SEQ ID NO: 28 - protein, 60 aa, Artificial Sequence
  SH3 domain
PQCKALYDFEVKDKEADKDCLPFAKDDVLTVIRRVDENWAEGMLADKIGIFPISYVEFNS

SEQ ID NO: 29 - protein, 58 aa, Artificial Sequence
  SH3 domain
SVYVAIYPYTPRKEDELELRKGEMFLVFERCQDGWFKGTSMHTSKIGVFPGNYVAPVT

SEQ ID NO: 30 - protein, 57 aa, Artificial Sequence
  SH3 domain
ERHRVVVSYPPQSEAELELKEGDIVFVHKKREDGWFKGTLQRNGKTGLFPGSFVENI

SEQ ID NO: 31 - DNA, 121 bp, Artificial Sequence
  RING domain
tgtccggtgtgtctagagcgccttgatgcttctgcgaaggtcttgccttgccagcatacgttttgcaagcgatgtttgctggggatcgtaggttctcgaaatgaactcagatgtcccgagt

SEQ ID NO: 32 - DNA, 165 bp, Artificial Sequence
  SH3 domain
ccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattatt

SEQ ID NO: 33 - DNA, 177 bp, Artificial Sequence
  SH3 domain
cctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaac

SEQ ID NO: 34 - DNA, 171 bp, Artificial Sequence
  SH3 domain
agtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtc

SEQ ID NO: 35 - DNA, 169 bp, Artificial Sequence
  SH3 domain
gaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaaca

SEQ ID NO: 36 - DNA, 21 bp, Artificial Sequence
  target sequence
aagtccaaaggttccggagac

SEQ ID NO: 37 - protein, 4 aa, Artificial Sequence
  chemically synthesized
  VARIANT 2: Xaa = Thr or Ser
PXAP

SEQ ID NO: 38 - protein, 5 aa, Artificial Sequence
  chemically synthesized
PFRDY

SEQ ID NO: 39 - protein, 7 aa, Artificial Sequence
  chemically synthesized
RPEPTAP

SEQ ID NO: 40 - protein, 7 aa, Artificial Sequence
  chemically synthesized
RQGPKEP

SEQ ID NO: 41 - protein, 9 aa, Artificial Sequence
  chemically synthesized
RQGPKEPFR

SEQ ID NO: 42 - protein, 9 aa, Artificial Sequence
  chemically synthesized
RPEPTAPEE

SEQ ID NO: 43 - protein, 7 aa, Artificial Sequence
  chemically synthesized
RPLPVAP

SEQ ID NO: 44 - DNA, 53 bp, Artificial Sequence
  scrambled human POSH oligonucleotide
cacacactgccgtcaactgttcaagagacagttgacggcagtgtgtgtttttt

SEQ ID NO: 45 - DNA, 61 bp, Artificial Sequence
  scrambled human POSH oligonucleotide
aattaaaaaacacacactgccgtcaactgtctcttgaacagttgacggcagtgtgtgggcc

SEQ ID NO: 46 - DNA, 50 bp, Artificial Sequence
  oligonucleotide encoding RNAi against human POSH
aacagaggccttggaaacctggaagcttgcaggtttccaaggcctctgtt

SEQ ID NO: 47 - DNA, 54 bp, Artificial Sequence
  oligonucleotide encoding RNAi against human POSH
gatcaacagaggccttggaaacctgcaagcttccaggtttccaaggcctctgtt

SEQ ID NO: 48 - DNA, 29 bp, Artificial Sequence
  primer
ggcccactagtcaaggtcgggcaggaaga

SEQ ID NO: 49 - DNA, 48 bp, Artificial Sequence
  primer
gccgaattcaaaaaggatccggcgatatccggtgtttcgtcctttcca

SEQ ID NO: 50 - protein, 836 aa, Artificial Sequence
  POSH fragment
RTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 51 - DNA, 1502 bp, Homo sapiens
agagacgtgaacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 52 - DNA, 2217 bp, Homo sapiens
gctgtgtggcccaggcttttctcaaactcctgagggcaagcgatcctcccacctcagcctcctgagtagctgggactacaggcatgtgccactagacctggctctaaagacatatatgacacacgaaaccatttatttttcatttcacaatgtttattcacatatatggtattagtattctaatgtagtgatgcactctaaatttgcattatatttcctagaacatctgaacagagcataggaaattccctattttgccattatcagttctaacaaaaatcttaaaagcactttatcatttcatttccctgcactgtaatttttttaaatgatcaaaaacagtatcataccaaggcttacttatattggaatactattttagaaagttgtgggctgggttgtatttataaatcttgttggtcagatgtctgcaatgagtaaatttagcaccattatcaggaagctttctcaccaatgacaacttcattggaagattttaatgaaagtgtagcatactctagggaaaaaatatgaatattttagcatctatgtattgaaaattatgttgaataaatgtcagactattttttacataacgttgcttctgtttaattttgtcacgttcagaggtggggggtaggagatgtaagcccttgacagcaaaataattccttttgcttgatttcagacagttgcatcagctcctttgttctgtgttcatgttacacttatttaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgacaaaaaaaaaaaaaaaaaa

SEQ ID NO: 53 - DNA, 1684 bp, Homo sapiens
ggccacctcaaggcccacctgagccgcgtctaccccgagcgtccgcgtccagaggaccagaggttaatttattctgggaagctgttgttggatcaccaatgtctcagggacttgcttccaaaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaaaaaaaaa

SEQ ID NO: 54 - DNA, 1878 bp, Homo sapiens
ccacgcgtccgggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaa

SEQ ID NO: 55 - DNA, 1864 bp, Homo sapiens
aacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaaaaaaaaaaaaaaaaaa

SEQ ID NO: 56 - DNA, 1871 bp, Homo sapiens
gaactgtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaaaaaaaaaaaaaaaaaaaa

SEQ ID NO: 57 - DNA, 1865 bp, Homo sapiens
agagacgtgaactgtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctg

SEQ ID NO: 58 - DNA, 1884 bp, Homo sapiens
gacgtgaacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaaaaaaaaa

SEQ ID NO: 59 - DNA, 1860 bp, Homo sapiens
cgtgaacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtgtttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtaagatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctg

SEQ ID NO: 60 - DNA, 1884 bp, Homo sapiens; identical to SEQ ID NO: 58

SEQ ID NO: 61 - protein, 232 aa, Homo sapiens
MESETEPEPVTLLVKSPNQRHRDLELSGDRGWSVGHLKAHLSRVYPERPRPEDQRLIYSGKLLLDHQCLRDLLPKEKRHVLHLVCNVKSPSKMPEINAKVAESTEEPAGSNRGQYPEDSSSDGLRQREVLRNLSSPGWENISRHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 62 - protein, 209 aa, Homo sapiens
MQYLAATAASGAFVPPPSAQEIPVVSAPAPAPIHNQFPAENQPANQNAAPQVVVNPGANQNLRMNAQGGPIVEEDDEINRDWLDWTYSAATFSVFLSILYFYSSLSRFLMVMGATVVMYLHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 63 - protein, 356 aa, Homo sapiens
GHLKAHLSRVYPERPRPEDQRLIYSGKLLLDHQCLRDLLPKEKRHVLHLVCNVKSPSKMPEINAKVAESTEEPAGSNRGQYPEDSSSDGLRQREVLRNLSSPGWENISRPEAAQQAFQGLGPGFSGYTPYGWLQLSWFQQIYARQYYMQYLAATAASGAFVPPPSAQEIPVVSAPAPAPIHNQFPAENQPANQNAAPQVVVNPGANQNLRMNAQGGPIVEEDDEINRDWLDWTYSAATFSVFLSILYFYSSLSRFLMVMGATVVMYLHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 64 - protein, 391 aa, Homo sapiens
MESETEPEPVTLLVKSPNQRHRDLELSGDRGWSVGHLKAHLSRVYPERPRPEDQRLIYSGKLLLDHQCLRDLLPKQEKRHVLHLVCNVKSPSKMPEINAKVAESTEEPAGSNRGQYPEDSSSDGLRQREVLRNLSSPGWENISRPEAAQQAFQGLGPGFSGYTPYGWLQLSWFQQIYARQYYMQYLAATAASGAFVPPPSAQEIPVVSAPAPAPIHNQFPAENQPANQNAAPQVVVNPGANQNLRMNAQGGPIVEEDDEINRDWLDWTYSAATFSVFLSILYFYSSLSRFLMVMGATVVMYLHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 65 - DNA, 1857 bp, Rat
aagacaccaagtgtcgttgtggggtcgcagacggctgcgtcgccgcccgttcggcatccctgagcgcagtcgagcctccagcgccgcagacatggagcccgagccacagcccgagccggtcacgctgctggtgaagagccccaatcagcgccaccgcgacttggagctgagtggcgaccgcggttggagtgtgagtcgcctcaaggcccacctgagccgagtctaccccgaacgcccgcgcccagaggaccagaggttaatttattctgggaagctgctgttggatcaccaatgtctccaagacttgcttccaaagcaggaaaagcgacatgttttgcacctcgtgtgcaatgtgaggagtccctcaaaaaagccagaagccagcacaaagggtgctgagtccacagagcagccggacaacactagtcaggcacagtatcctggggattcctcaagcgatggcttacgggaaagggaagtccttcggaaccttcctccctctggatgggagaacgtctctaggcctgaagccgtccagcagactttccaaggcctcgggcccggcttctctggctacaccacctacgggtggctgcagctctcctggttccagcagatctatgcaagacagtactacatgcaatacttggctgccactgctgcttcaggagcttttggccctacaccaagtgcacaagaaatacctgtggtctctacaccggctcccgcccctatacacaaccagtttccggcagaaaaccagccggccaatcagaatgcagccgctcaagcggttgttaatcccggagccaatcagaacttgcggatgaatgcacaaggcggccctctggtggaagaagatgatgagataaaccgagactggttggattggacctactcagcagcgacattttccgttttcctcagcattctttacttctactcctccctgagcagattcctcatggtcatgggcgccaccgtagtcatgtacctgcaccacgtcgggtggtttccattcagacagaggccagttcagaacttcccagatgacggtccccctcaggaagctgccaaccaggaccccaacaataacctccagggaggtttggaccctgaaatggaagaccccaaccgcctccccgtaggccgtgaagtgctggaccctgagcataccagcccctcgttcatgagcacagcatggctagtcttcaagactttctttgcctctcttcttccggaaggcccaccagccctagcaaactgatggcccctgtgctctgttgctggaggctttcacagcttggactggatcgtcccctggcgtggactcgagagagtcattgaaaacccacaggatgacgatgtgcttctgtgccaagcaaaagcacaaactaagacatgaagccgtggtacaaactgaacagggcccctcatgtcgttattctgaagagctttaatgtatactgtatgtagtctcataggcactgtaaacagaaggcccagggtcgcatgttctgcctgagcacctccccagacgtgtgtgcatgtgtgccgtacatggaagtcatagacgtgtgtgcatgtgtgctctacatggaagtcatagatgcagaaacggttctgctggttcgatttgattcctgttggaatgttgcaattacactaagtgtactactttatataatcagtgacttgctagacatgttagcaggacttttctaggagagacttattgtatcattgctttttaaaacgcagtgcttacttactgagggcggcgacttggcacaggtaaagcctttgccgggttttctgttcaataaagttttgctatgaacgacaaaaaaaaaaaaa

SEQ ID NO: 66 - protein, 391 aa, Rat
MEPEPQPEPVTLLVKSPNQRHRDLELSGDRGWSVSRLKAHLSRVYPERPRPEDQRLIYSGKLLLDHQCLQDLLPKQEKRHVLHLVCNVRSPSKKPEASTKGAESTEQPDNTSQAQYPGDSSSDGLREREVLRNLPPSGWENVSRPEAVQQTFQGLGPGFSGYTTYGWLQLSWFQQIYARQYYMQYLAATAASGAFGPTPSAQEIPVVSTPAPAPIHNQFPAENQPANQNAAAQAVVNPGANQNLRMNAQGGPLVEEDDEINRDWLDWTYSAATFSVFLSILYFYSSLSRFLMVMGATVVMYLHHVGWFPFRQRPVQNFPDDGPPQEAANQDPNNNLQGGLDPEMEDPNRLPVGREVLDPEHTSPSFMSTAWLVFKTFFASLLPEGPPALAN

SEQ ID NO: 67 - DNA, 1871 bp, Mouse
aaagacgccaagtgtcgttgtgtggtctcagacggctgcgtcgccgcccgttcggcatccctgagcgcagtcgagccgccagcgacgcagacatggagcccgagccacagcccgagccggtcacgctgctggtgaagagtcccaatcagcgccaccgcgacttggagctgagtggcgaccgcagttggagtgtgagtcgcctcaaggcccacctgagccgagtctaccccgagcgcccgcgtccagaggaccagaggttaatttattctgggaagctgctgttggatcaccagtgtctccaagatttgcttccaaagcaggaaaagcgacatgttttgcaccttgtgtgcaatgtgaagaatccctccaaaatgccagaaaccagcacaaagggtgctgaatccacagagcagccggacaactctaatcagacacagcatcctggggactcctcaagtgatggtttacggcaaagagaagttcttcggaacctttctccctccggatgggagaacatctctaggcctgaggctgtccagcagactttccaaggcctggggcctggcttctctggctacacaacgtatgggtggctgcagctctcctggttccagcagatctatgcaaggcagtactacatgcaatacttagctgccactgctgcatcaggaacttttgtcccgacaccaagtgcacaagagatacctgtggtctctacacctgctccggctcctatacacaaccagtttccggcagaaaaccagccggccaatcagaatgcagctgctcaagcggttgtcaatcccggagccaatcagaacttgcggatgaatgcacaaggtggccccctggtggaggaagatgatgagataaaccgagactggttggattggacctattccgcagcgacgttttctgttttcctcagcatcctttacttctactcctcgctgagcagatttctcatggtcatgggtgccactgtagtcatgtacctgcaccacgtcgggtggtttccgttcagacagaggccagttcagaacttcccggatgatggtggtcctcgagatgctgccaaccaggaccccaacaataacctccagggaggtatggacccagaaatggaagaccccaaccgcctccccccagaccgcgaagtgctggaccctgagcacaccagcccctcgtttatgagcacagcatggctagtcttcaagactttctttgcctctcttcttccagaaggcccaccagccctagccaactgatggcccttgtgctctgtcgctggtggctttgacagctcggactggatcgtctggctccggctccttttcctcccctggcgtggactcgacagagtcattgaaaacccacaggatgacatgtgcttctgtgccaagcaaaagcacaaactaagacatgaagccgtggtacaaactgaacagggcccctcatgtcgttattctgaagagctttaatgtatactgtatgtagtttcataggcactgtaagcagaaggcccagggtcgcatgttctgcctgagcacctccccagatgtgtgtgcatgtgtgctgtacatggaagtcatagacgtgtgtgcatgtgtgctctacatggaagtcatagatgcagaaacggttctgctggttcgatttgattcctgttggaatgttcaaattacactaagtgtactactttatataatcagtgaattgctagacatgttagcaggacttttctaggagagacttatgtataattgctttttaaaatgcagtgctttcctttaaaccgagggtggcgacttggcagaggtaaaacctttgccgagttttctgttcaataaagttttgctatgaatgactgt

SEQ ID NO: 68 - protein, 391 aa, Mouse
MEPEPQPEPVTLLVKSPNQRHRDLELSGDRSWSVSRLKAHLSRVYPERPRPEDQRLIYSGKLLLDHQCLQDLLPKQEKRHVLHLVCNVKNPSKMPETSTKGAESTEQPDNSNQTQHPGDSSSDGLRQREVLRNLSPSGWENISRPEAVQQTFQGLGPGFSGYTTYGWLQLSWFQQIYARQYYMQYLAATAASGTFVPTPSAQEIPVVSTPAPAPIHNQFPAENQPANQNAAAQAVVNPGANQNLRMNAQGGPLVEEDDEINRDWLDWTYSAATFSVFLSILYFYSSLSRFLMVMGATVVMYLHHVGWFPFRQRPVQNFPDDGGPRDAANQDPNNNLQGGMDPEMEDPNRLPPDREVLDPEHTSPSFMSTAWLVFKTFFASLLPEGPPALAN

SEQ ID NO: 69 - DNA, 21 bp, Artificial Sequence
  siRNA
gggaaguucuucggaaccutt

SEQ ID NO: 70 - DNA, 21 bp, Artificial Sequence
  siRNA
ttcccuucaagaagccuugga